(19) United States
(12) Patent Application Publication
(10) Pub. No.: US 2007/0225334 A1
(43) Pub. Date: Sep. 27, 2007

(54) Methods for treating cognitive and other disorders

(75) Inventor: Sharon Rosenzweig-Lipson, East Brunswick, NJ (US)

Correspondence Address: CHOATE, HALL & STEWART LLP/WYETH, PATENT GROUP, TWO INTERNATIONAL PLACE, BOSTON, MA, 02110, US

(73) Assignee: Wyeth, Madison, NJ (US)

(21) Appl. No.: 11/726,941

(22) Filed: Mar. 23, 2007

Related U.S. Application Data
(60) Provisional application No. 60/785,778, filed on Mar. 24, 2006.

(51) Int. Cl. A61K 31/4433; A61K 31/381; A61K 31/343

(52) U.S. Cl. 514/337; 514/444; 514/469

(57) ABSTRACT

This invention provides methods and pharmaceutical compositions for treating cognitive disorders such as learning disorders, ADD/ADHD, and other disorders.

### CROSS-REFERENCE TO RELATED APPLICATIONS

This application claims priority to U.S. provisional patent application Ser. No. 60/785,778, filed Mar. 24, 2006, the entirety of which is hereby incorporated herein by reference.

### FIELD OF THE INVENTION

The present invention relates to compounds useful in treating disorders associated with 5HT_{2C} modulation.

### BACKGROUND OF THE INVENTION

Cognition is the ability of one's brain to think, to process and store information, and to solve problems. Cognitive abilities include memory, language, attention, perception, and reasoning. Cognition is a high level of behavior unique to humans. Many cognitive disorders affect the elderly, such as Alzheimer's disease and memory deficit. However, there are also many cognitive disorders that affect children, adolescents, and young adults.

There are a variety of brain abnormalities which prevent infants and children from developing normal social and/or cognitive skills. Disorders of the basic psychological processes can affect the way a child/adolescent learns. Many children/adolescents with learning disabilities have average or above average intelligence. However, learning disabilities may cause difficulties in listening, thinking, talking, reading, writing, spelling, or arithmetic. Such learning disabilities include perceptual handicaps, dyslexia, dyscalculia, dysgraphia and developmental aphasia.

Attention deficit disorders (ADD), also known as attention deficit hyperactivity disorder (ADHD), is a well-known cognitive disorder that affects children and adults alike. It is estimated that between 3% and 8% of all children have ADD. ADD is characterized by symptoms such as hyperactivity, impulsiveness, distractibility and difficulty sustaining attention for periods of time. Symptoms may be different in each person with ADD. Some may have more of a problem with inability to focus, while others may have the most difficult time with impulsiveness. Medications are available to treat ADD, often in the form of stimulants such as Ritalin, Adderal, and Strattera, to name a few. However, there are certain side effects associated with such treatments, including decreased appetite and problems sleeping.

Accordingly, there remains a need to develop treatments for the variety of cognitive disorders affecting patients of all ages.

### SUMMARY OF THE INVENTION

The present invention provides methods for treating a cognitive disorder in a mammal, including methods for treating a learning disorder, an attention deficit disorder, an impulsivity disorder, or a behavioral addiction, among others. In particular, according to the present invention, compounds of formula I: 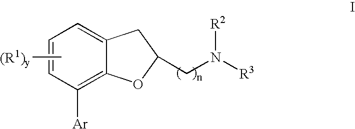
or a pharmaceutically acceptable salt thereof, wherein:
n is one or two;
each of R² and R³ is independently hydrogen, methyl, ethyl, 2-fluoroethyl, 2,2-difluoroethyl or cyclopropyl;
each R¹ is independently hydrogen, halogen, OH, lower alkyl, lower alkoxy, lower haloalkyl, lower haloalkoxy, or CN;
Ar is thienyl, furyl, pyridyl, or phenyl, wherein Ar is optionally substituted with one or more R^{x} substituents;
each R^{x} is independently selected from halogen, OH, lower alkyl, lower alkoxy, lower haloalkyl, lower haloalkoxy, or CN; and
y is 0-3,
   which are highly selective agonists, or partial agonists, of the 5HT_{2C} receptor, are useful in the treatment of cognitive and other disorders as described herein.

The present invention provides, among other things, methods of a cognitive disorder by administrating to an individual in need thereof a pharmaceutically effective amount of a compound of formula I. The invention also provides pharmaceutical compositions of compounds of formula I formulated and dosed for treatment of a cognitive disorder, as well as combinations of compounds of formula I with one or more other agents useful in the treatment of cognitive and/or other disorders or diseases suffered by individuals with cognitive disorders. Yet other aspects of the present invention will be clear to those of ordinary skill in the art upon review of the present specification and claims.

### BRIEF DESCRIPTION OF THE DRAWING

FIG. 1 shows the effects of Compound 1 on adjunctive and normal drinking behavior in Sprague-Dawley rats.

FIG. 2 shows the effects of Compound 1 on acetylcholine in medial prefrontal cortex.

FIG. 3 shows the effects of Compound 1 on glutamate in medial prefrontal cortex.

### DETAILED DESCRIPTION OF CERTAIN EMBODIMENTS OF THE INVENTION

1. Compounds

Compounds useful for treating cognitive, and other disorders, according to the present invention include compounds of formula I: 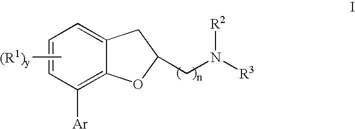
or a pharmaceutically acceptable salt thereof, wherein:
n is one or two;
each of R² and R³ is independently hydrogen, methyl, ethyl, 2-fluoroethyl, 2,2-difluoroethyl or cyclopropyl; each R¹ is independently hydrogen, halogen, OH, lower alkyl, lower alkoxy, lower haloalkyl, lower haloalkoxy, or CN;
Ar is thienyl, furyl, pyridyl, or phenyl, wherein Ar is optionally substituted with one or more R^{x} subsituents;
each R^{x} is independently selected from halogen, OH, lower alkyl, lower alkoxy, lower haloalkyl, lower haloalkoxy, or CN; and
y is 0-3,

The term “lower alkyl,” as used herein, refers to a hydrocarbon chain having up to 4 carbon atoms, preferably 1 to 3 carbon atoms, and more preferably 1 to 2 carbon atoms. The term “alkyl” includes, but is not limited to, straight and branched chains such as methyl, ethyl, n-propyl, isopropyl, n-butyl, isobutyl, sec-butyl, or t-butyl.

The term “alkoxy,” as used herein, refers to the group —OR, wherein R is a lower alkyl group.

The terms “halogen” or “halo,” as used herein, refer to chlorine, bromine, fluorine or iodine.

The term “haloalkyl,” as used herein, or as part of a moiety such as “haloalkoxy” refers to an alkyl group, as defined herein, that has one or more halogen substituents. In certain embodiment, every hydrogen atom on said alkyl group is replaced by a halogen atom. Such haloalkyl groups include —CF₃. Such haloalkoxy groups include —OCF₃.

It will be appreciated by those of ordinary skill in the art that reference to a compound herein is intended to include reference to any and all related forms such as stereoisomers, polymorphs, hydrates, etc. Also, compounds may be provided as pro-drugs or other forms converted into the active agent during manufacture, processing, formulation, delivery, or in the body.

The term “pharmaceutically acceptable salts” or “pharmaceutically acceptable salt” refers to salts derived from treating a compound of formula I with an organic or inorganic acid such as, for example, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, or similarly known acceptable acids. In certain embodiments, the present invention provides the hydrochloride salt of a compound of formula I.

As defined generally above, each of the R² and R³ groups of formula I is independently hydrogen, methyl, ethyl, 2-fluoroethyl, 2,2-difluoroethyl or cyclopropyl. In certain embodiments, one of the R² and R³ groups of formula I is hydrogen and the other R² or R³ group of formula I is hydrogen, methyl, ethyl, 2-fluoroethyl, 2,2-difluoroethyl or cyclopropyl. In other embodiments, neither of the R² and R³ groups of formula I is hydrogen. In still other embodiments, both of the R² and R³ groups of formula I are hydrogen.
As defined generally above, each R¹ group of formula I is independently hydrogen, halogen, OH, lower alkyl, lower alkoxy, lower haloalkyl, lower haloalkoxy, or CN. In certain embodiments, each R¹ group of formula I is hydrogen. In other embodiments, at least one of R¹ group of formula I is halogen. In still other embodiments, y is 1 and R¹ is halogen.

According to another embodiment, y is 1 and R¹ is at the 5-position of the dihydrobenzofuran ring of formula I, thus forming a compound of formula Ia: 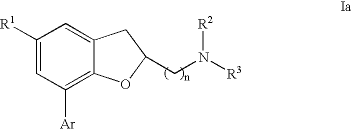
or a pharmaceutically acceptable salt thereof, wherein each of R¹, R², R³, Ar, and n are as defined above for compounds of formula I and in classes and subclasses as described above and herein.

According to yet another embodiment, y is 1 and R¹ is at the 6-position of the dihydrobenzofuran ring of formula I, thus forming a compound of formula Ia′: 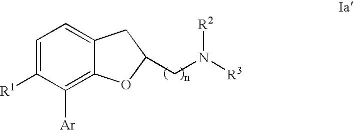
or a pharmaceutically acceptable salt thereof, wherein each of R¹, R², R³, Ar, and n are as defined above for compounds of formula I and in classes and subclasses as described above and herein.

As defined generally above, the Ar group of formula I is thienyl, furyl, pyridyl, or phenyl, wherein Ar is optionally substituted with one or more subsituents independently selected from halogen, OH, lower alkyl, lower alkoxy, haloalkyl, haloalkoxy, or CN. In certain embodiments, the Ar group of formula I is unsubstituted phenyl. In other embodiments, the Ar group of formula I is phenyl with at least one substituent in the ortho position. In other embodiments, the Ar group of formula I is phenyl with at least one substituent in the ortho position selected from halogen, lower alkyl, lower alkoxy, or trifluoromethyl. According to another aspect the present invention provides a compound of formula I wherein Ar is phenyl di-substituted in the ortho and meta positions with independently selected halogen lower alkyl, or lower alkoxy. Yet another aspect of the present invention provides a compound of formula I wherein Ar is phenyl di-substituted in the ortho and para positions with independently selected halogen lower alkyl, or lower alkoxy. In other embodiment, the present invention provides a compound of formula I wherein Ar is phenyl di-substituted in the ortho positions with independently selected halogen lower alkyl, or lower alkoxy. Exemplary substituents on the phenyl moiety of the Ar group of formula I include OMe, fluoro, chloro, methyl, and trifluoromethyl.

In certain embodiments, the present invention provides a compound of formula Ia′ wherein Ar is phenyl with one substituent in the ortho position selected from halogen, lower alkyl, lower alkoxy, or trifluoromethyl.

According to one embodiment, Ar is phenyl substituted with one R^{x} substituent in the ortho-position, thus forming a compound of formula Ib, or with an R^{x} substituent in both ortho-positions, thus forming a compound of formula Ic: 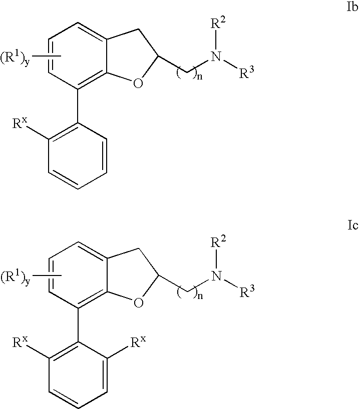
or a pharmaceutically acceptable salt thereof, wherein each R¹, R², R³, R^{x}, y and n are as defined above for compounds of formula I and in classes and subclasses as described above and herein.

In certain embodiments, the Ar group of formula I is selected from the following:

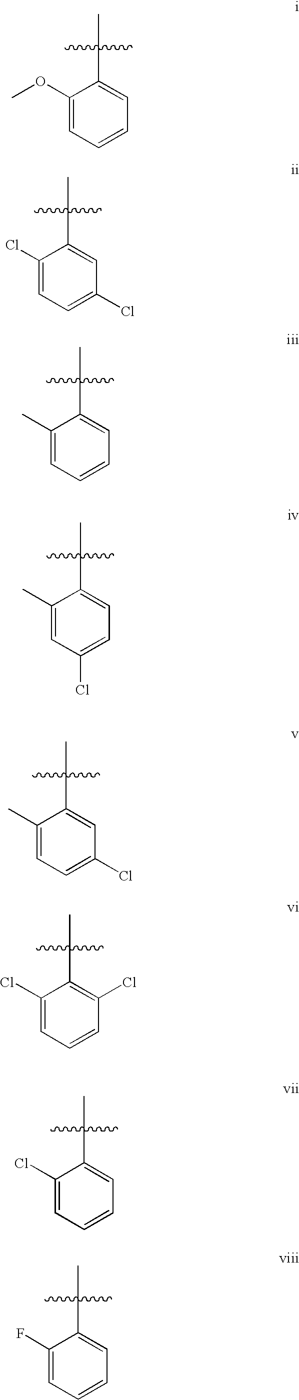

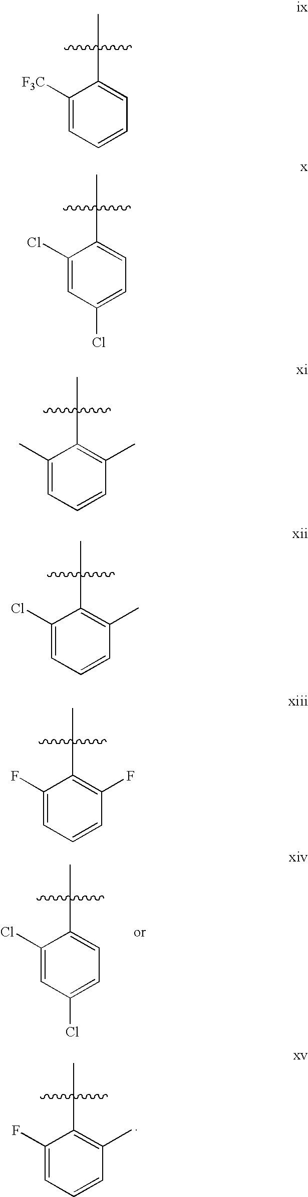

According to yet another embodiment, the present invention provides a compound of formula Id or Ie: 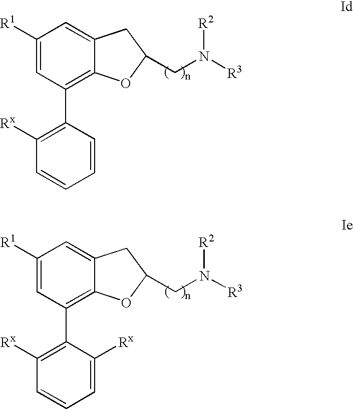
or a pharmaceutically acceptable salt thereof, wherein each R¹, R², R³, R^{x}, and n are as defined above for compounds of formula I and in classes and subclasses as described above and herein.

According to another embodiment, the present invention provides a compound of formula If or Ig: 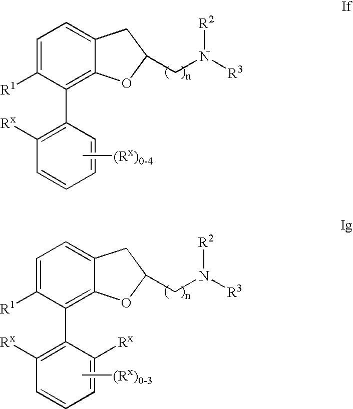
or a pharmaceutically acceptable salt thereof, wherein each R¹, R², R³, R^{x}, and n are as defined above for compounds of formula I and in classes and subclasses as described above and herein.

In certain embodiments, the present invention provides a compound of formula Ih or Ii: 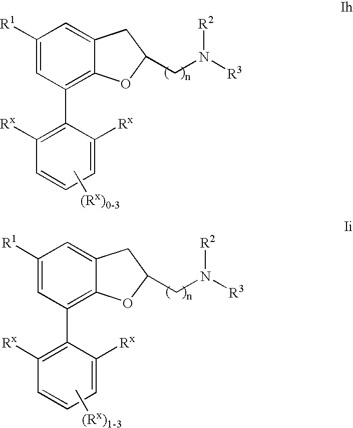
or a pharmaceutically acceptable salt thereof, wherein each R¹, R², R³, R^{x}, and n are as defined above for compounds of formula I and in classes and subclasses as described above and herein.

Compounds of the present invention contain asymmetric carbon atoms and thus give rise to stereoisomers, including enantiomers and diastereomers. Accordingly, it is contemplated that the present invention relates to all of these stereoisomers, as well as to mixtures of the stereoisomers. Throughout this application, the name of the product of this invention, where the absolute configuration of an asymmetric center is not indicated, is intended to embrace the individual stereoisomers as well as mixtures of stereoisomers. In certain embodiments of the invention, compounds having an absolute (R) configuration are preferred.

In certain embodiments, the present invention provides a compound of formula IIa or IIb: 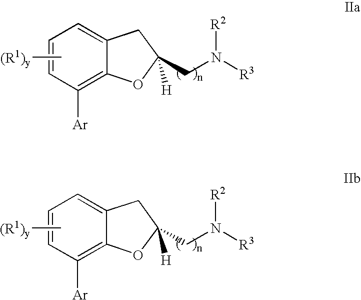
or a pharmaceutically acceptable salt thereof, wherein each R¹, R², R³, Ar, y and n are as defined above for compounds of formula I and in classes and subclasses as described above and herein.

According to another embodiment, the present invention provides a compound of formula IIc or IId: 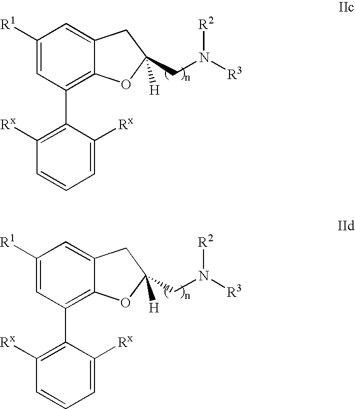
or a pharmaceutically acceptable salt thereof, wherein each R¹, R², R³, R^{x}, and n are as defined above for compounds of formula I and in classes and subclasses as described above and herein.

In certain embodiments, the present invention provides a compound of formula IIIa or IIIb: 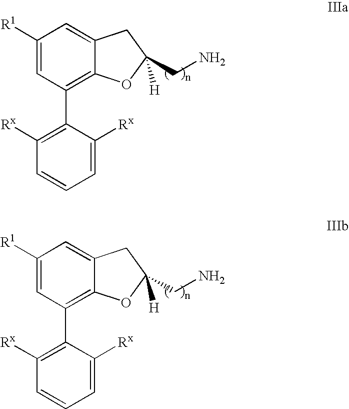
or a pharmaceutically acceptable salt thereof, wherein each R¹ and R^{x} are as defined above for compounds of formula I and in classes and subclasses as described above and herein.

In other embodiments, the present invention provides a compound of formula IIIc or IIId: 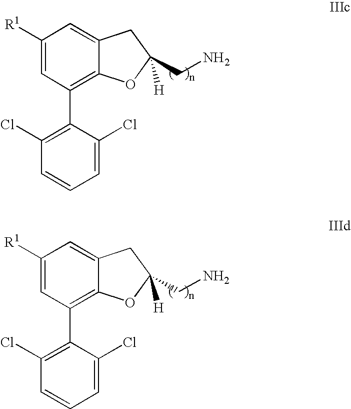
or a pharmaceutically acceptable salt thereof, wherein R¹ is as defined above for compounds of formula I and in classes and subclasses as described above and herein.

According to another aspect, methods of the present invention are carried out with the following compound: 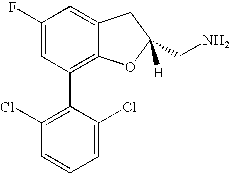
or a pharmaceutically acceptable salt thereof.

Where an enantiomer is preferred, it may, in some embodiments be provided substantially free of the corresponding enantiomer. Thus, an enantiomer substantially free of the corresponding enantiomer refers to a compound which is isolated or separated via separation techniques or prepared free of the corresponding enantiomer. “Substantially free,” as used herein, means that the compound is made up of a significantly greater proportion of one enantiomer. In certain embodiments the compound is made up of at least about 90% by weight of a preferred enantiomer. In other embodiments of the invention, the compound is made up of at least about 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

It is further recognized that atropisomers of the present compounds may exit. The present invention thus encompasses atropisomeric forms of compounds of formula I as defined above, and in classes and subclasses described above and herein.

Exemplary compounds useful for methods of the present invention are set forth in Table 1, below.

TABLE 1
Exemplary Compounds of Formula I

(±)-1-{7-[3,5-bis(trifluoromethyl)phenyl]-2,3-dihydro-1-benzofuran-2-yl}methanamine,
(±)-1-[7-(3-chloro-4-fluorophenyl)-2,3-dihydro-1-benzofuran-2-yl]methanamine,
(±)-1-[7-(3,5-dichlorophenyl)-2,3-dihydro-1-benzofuran-2-yl]methanamine,
(±)-1-(7-phenyl-2,3-dihydro-1-benzofuran-2-yl)methanamine,
(+)-(1-(7-phenyl-2,3-dihydro-1-benzofuran-2-yl)methanamine,
(−)-1-(7-phenyl-2,3-dihydro-1-benzofuran-2-yl)methanamine,
(±)-1-[7-(3-methylphenyl)-2,3-dihydro-1-benzofuran-2-yl]methanamine,
(+)-1-[7-(3-methylphenyl)-2,3-dihydro-1-benzofuran-2-yl]methanamine,
(−)-1-[7-(3-methylphenyl)-2,3-dihydro-1-benzofuran-2-yl]methanamine,
(±)-1-(7-thien-3-yl-2,3-dihydro-1-benzofuran-2-yl)methanamine,
(+)-1-(7-thien-3-yl-2,3-dihydro-1-benzofuran-2-yl)methanamine,
(−)-1-(7-thien-3-yl-2,3-dihydro-1-benzofuran-2-yl)methanamine,
(±)-1-[7-(2-methylphenyl)-2,3-dihydro-1-benzofuran-2-yl]methanamine,
(+)-1-[7-(2-methylphenyl)-2,3-dihydro-1-benzofuran-2-yl]methanamine,
(−)-1-[7-(2-methylphenyl)-2,3-dihydro-1-benzofuran-2-yl]methanamine,
(±)-1-[7-(2-fluorophenyl)-2,3-dihydro-1-benzofuran-2-yl]methanamine,
(+)-1-[7-(2-fluorophenyl)-2,3-dihydro-1-benzofuran-2-yl]methanamine,
(−)-1-[7-(2-fluorophenyl)-2,3-dihydro-1-benzofuran-2-yl]methanamine,
(±)-1-{7-[2-(trifluoromethyl)phenyl]-2,3-dihydro-1-benzofuran-2-yl}methanamine,
(−)-1-{7-[2-(trifluoromethyl)phenyl]-2,3-dihydro-1-benzofuran-2-yl}methanamine,
(+)-1-{7-[2-(trifluoromethyl)phenyl]-2,3-dihydro-1-benzofuran-2-yl}methanamine,
(±)-1-[7-(2,6-dimethylphenyl)-2,3-dihydro-1-benzofuran-2-yl]methanamine,
(−)-1-[7-(2,6-dimethylphenyl)-2,3-dihydro-1-benzofuran-2-yl]methanamine,
(+)-1-[7-(2,6-dimethylphenyl)-2,3-dihydro-1-benzofuran-2-yl]methanamine,
(±)-1-[7-(2-methoxyphenyl)-2,3-dihydro-1-benzofuran-2-yl]methanamine,
(±)-1-[7-(2-chlorophenyl)-2,3-dihydro-1-benzofuran-2-yl]methanamine,
(±)-1-[7-(3-fluorophenyl)-2,3-dihydro-1-benzofuran-2-yl]methanamine,
(±)-1-[7-(3-chlorophenyl)-2,3-dihydro-1-benzofuran-2-yl]methanamine,
(+)-1-[7-(3-chlorophenyl)-2,3-dihydro-1-benzofuran-2-yl]methanamine,
(−)-1-[7-(3-chlorophenyl)-2,3-dihydro-1-benzofuran-2-yl]methanamine,
(±)-1-[7-(3-methoxyphenyl)-2,3-dihydro-1-benzofuran-2-yl]methanamine,
(±)-1-{7-[3-(trifluoromethyl)phenyl]-2,3-dihydro-1-benzofuran-2-yl}methanamine,
(±)-1-[7-(4-methylphenyl)-2,3-dihydro-1-benzofuran-2-yl]methanamine,
(+)-1-[7-(4-methylphenyl)-2,3-dihydro-1-benzofuran-2-yl]methanamine,
(−)-1-[7-(4-methylphenyl)-2,3-dihydro-1-benzofuran-2-yl]methanamine,
(±)-1-[7-(4-fluorophenyl)-2,3-dihydro-1-benzofuran-2-yl]methanamine,
(+)-1-[7-(4-fluorophenyl)-2,3-dihydro-1-benzofuran-2-yl]methanamine,
(−)-1-[7-(4-fluorophenyl)-2,3-dihydro-1-benzofuran-2-yl]methanamine,
(±)-1-[7-(4-chlorophenyl)-2,3-dihydro-1-benzofuran-2-yl]methanamine,
(+)-1-[7-(4-chlorophenyl)-2,3-dihydro-1-benzofuran-2-yl]methanamine,
(−)-1-[7-(4-chlorophenyl)-2,3-dihydro-1-benzofuran-2-yl]methanamine,
(±)-1-[7-(4-methoxyphenyl)-2,3-dihydro-1-benzofuran-2-yl]methanamine,
(+)-1-[7-(4-methoxyphenyl)-2,3-dihydro-1-benzofuran-2-yl]methanamine,
(−)-1-[7-(4-methoxyphenyl)-2,3-dihydro-1-benzofuran-2-yl]methanamine,
(±)-1-{7-[4-(trifluoromethyl)phenyl]-2,3-dihydro-1-benzofuran-2-yl}methanamine,
(±)-1-[7-(2,4-dichlorophenyl)-2,3-dihydro-1-benzofuran-2-yl]methanamine,
(±)-1-(5-chloro-7-phenyl-2,3-dihydro-1-benzofuran-2-yl)methanamine,
(+)-1-(5-chloro-7-phenyl-2,3-dihydro-1-benzofuran-2-yl)methanamine,
(−)-1-(5-chloro-7-phenyl-2,3-dihydro-1-benzofuran-2-yl)methanamine,
(±)-1-[5-chloro-7-(2-chlorophenyl)-2,3-dihydro-1-benzofuran-2-yl]methanamine,
(±)-1-[5-chloro-7-(3-methylphenyl)-2,3-dihydro-1-benzofuran-2-yl]methanamine,
(±)-1-[5-chloro-7-(3-chlorophenyl)-2,3-dihydro-1-benzofuran-2-yl]methanamine,
(±)-(5-chloro-7-thien-3-yl-2,3-dihydro-1-benzofuran-2-yl)methylamine,
(−)-(5-chloro-7-thien-3-yl-2,3-dihydro-1-benzofuran-2-yl)methylamine,
(+)-(5-chloro-7-thien-3-yl-2,3-dihydro-1-benzofuran-2-yl)methylamine,
(+)-N-[(5-chloro-7-thien-3-yl-2,3-dihydro-1-benzofuran-2-yl)methyl]-N-methylamine,
(−)-N-[(5-chloro-7-thien-3-yl-2,3-dihydro-1-benzofuran-2-yl)methyl]-N-methylamine,
(±)-1-[5-chloro-7-(2-methylphenyl)-2,3-dihydro-1-benzofuran-2-yl]methanamine,
(+)-1-[5-chloro-7-(2-methylphenyl)-2,3-dihydro-1-benzofuran-2-yl]methanamine,
(−)-1-[5-chloro-7-(2-methylphenyl)-2,3-dihydro-1-benzofuran-2-yl]methanamine,
(±)-1-(4-fluoro-7-phenyl-2,3-dihydro-1-benzofuran-2-yl)methanamine,
(±)-1-[4-fluoro-7-(2-methylphenyl)-2,3-dihydro-1-benzofuran-2-yl]methanamine,
(±)-1-[7-(2-chlorophenyl)-5-fluoro-2,3-dihydro-1-benzofuran-2-yl]methanamine,
(+)-1-[7-(2-chlorophenyl)-5-fluoro-2,3-dihydro-1-benzofuran-2-yl]methanamine,
(−)-1-[7-(2-chlorophenyl)-5-fluoro-2,3-dihydro-1-benzofuran-2-yl]methanamine,
(±)-1-(5-fluoro-7-phenyl-2,3-dihydro-1-benzofuran-2-yl)methanamine,
(±)-1-[5-fluoro-7-(2-methylphenyl)-2,3-dihydro-1-benzofuran-2-yl]methanamine,
(+)-1-[5-fluoro-7-(2-methylphenyl)-2,3-dihydro-1-benzofuran-2-yl]methanamine,
(−)-1-[5-fluoro-7-(2-methylphenyl)-2,3-dihydro-1-benzofuran-2-yl]methanamine,
(±)-1-[5-fluoro-7-(2-fluorophenyl)-2,3-dihydro-1-benzofuran-2-yl]methanamine,
(±)-1-{5-fluoro-7-[2-(trifluoromethyl)phenyl]-2,3-dihydro-1-benzofuran-2-yl}methanamine,
(±)-(4,5-difluoro-7-phenyl-2,3-dihydro-1-benzofuran-2-yl)methylamine,
(±)-1-[4,5-difluoro-7-(2-methylphenyl)-2,3-dihydro-1-benzofuran-2-yl]methanamine,
(±)-1-(5-chloro-2-methyl-7-phenyl-2,3-dihydro-1-benzofuran-2-yl)methanamine,
(±)-(5-chloro-2-methyl-7-thien-3-yl-2,3-dihydro-1-benzofuran-2-yl)methylamine,
(±)-(5-chloro-2-methyl-7-thien-2-yl-2,3-dihydro-1-benzofuran-2-yl)methylamine,
(+)-1-[7-(2-methoxyphenyl)-2,3-dihydro-1-benzofuran-2-yl]methanamine,
(−)-1-[7-(2-chlorophenyl)-2,3-dihydro-1-benzofuran-2-yl]methanamine,
(+)-1-[7-(2-chlorophenyl)-2,3-dihydro-1-benzofuran-2-yl]methanamine,
(+)-1-[7-(3-fluorophenyl)-2,3-dihydro-1-benzofuran-2-yl]methanamine,
(−)-1-[7-(3-fluorophenyl)-2,3-dihydro-1-benzofuran-2-yl]methanamine,
(+)-1-[7-(3-methoxyphenyl)-2,3-dihydro-1-benzofuran-2-yl]methanamine,
(−)-1-[7-(3-methoxyphenyl)-2,3-dihydro-1-benzofuran-2-yl]methanamine,
(−)-1-{7-[3-(trifluoromethyl)phenyl]-2,3-dihydro-1-benzofuran-2-yl}methanamine,
(+)-1-{7-[3-(trifluoromethyl)phenyl]-2,3-dihydro-1-benzofuran-2-yl}methanamine,
(+)-1-{7-[4-(trifluoromethyl)phenyl]-2,3-dihydro-1-benzofuran-2-yl}methanamine,
(−)-1-{7-[4-(trifluoromethyl)phenyl]-2,3-dihydro-1-benzofuran-2-yl}methanamine,
(±)-1-[7-(2,6-difluorophenyl)-2,3-dihydro-1-benzofuran-2-yl]methanamine,
(±)-1-[7-(2,6-dichlorophenyl)-2,3-dihydro-1-benzofuran-2-yl]methanamine,
(−)-1-[7-(2,6-dichlorophenyl)-2,3-dihydro-1-benzofuran-2-yl]methanamine,
(+)-1-[7-(2,6-dichlorophenyl)-2,3-dihydro-1-benzofuran-2-yl]methanamine,
(±)-1-[7-(2,4-dimethoxyphenyl)-2,3-dihydro-1-benzofuran-2-yl]methanamine,
(−)-1-[7-(2,4-dimethoxyphenyl)-2,3-dihydro-1-benzofuran-2-yl]methanamine,
(+)-1-[7-(2,4-dimethoxyphenyl)-2,3-dihydro-1-benzofuran-2-yl]methanamine,
(±)-1-[7-(2,4-difluorophenyl)-2,3-dihydro-1-benzofuran-2-yl]methanamine,
(+)-1-[7-(2,4-difluorophenyl)-2,3-dihydro-1-benzofuran-2-yl]methanamine,
(−)-1-[7-(2,4-difluorophenyl)-2,3-dihydro-1-benzofuran-2-yl]methanamine,
(−)-1-[7-(2,4-dichlorophenyl)-2,3-dihydro-1-benzofuran-2-yl]methanamine,
(+)-1-[7-(2,4-dichlorophenyl)-2,3-dihydro-1-benzofuran-2-yl]methanamine,
(−)-1-{5-fluoro-7-[2-(trifluoromethyl)phenyl]-2,3-dihydro-1-benzofuran-2-yl}methanamine,
(+)-1-{5-fluoro-7-[2-(trifluoromethyl)phenyl]-2,3-dihydro-1-benzofuran-2-yl}methanamine,
(±)-1-[7-(2,3-dimethylphenyl)-2,3-dihydro-1-benzofuran-2-yl]methanamine,
(±)-{[7-(2,3-dimethoxyphenyl)-2,3-dihydro-1-benzofuran-2-yl]methyl}amine,
(−)-{[(7-(2,3-dimethoxyphenyl)-2,3-dihydro-1-benzofuran-2-yl]methyl}amine,
(+)-{[(7-(2,3-dimethoxyphenyl)-2,3-dihydro-1-benzofuran-2-yl]methyl}amine,
(+)-{[7-(4-chloro-2-methylphenyl)-2,3-dihydro-1-benzofuran-2-yl]methyl}amine,
(−)-{[7-(4-chloro-2-methylphenyl)-2,3-dihydro-1-benzofuran-2-yl]methyl}amine,
(±)-{[7-(2,3-difluorophenyl)-2,3-dihydro-1-benzofuran-2-yl]methyl}amine,
(±)-{[7-(2,5-dimethylphenyl)-2,3-dihydro-1-benzofuran-2-yl]methyl}amine,
(±)-{[7-(2,5-dimethoxyphenyl)-2,3-dihydro-1-benzofuran-2-yl]methyl}amine,
(±)-{[7-(2,5-dichlorophenyl)-2,3-dihydro-1-benzofuran-2-yl]methyl}amine,
(+)-{[7-(2,5-dichlorophenyl)-2,3-dihydro-1-benzofuran-2-yl]methyl}amine,
(−)-{[7-(2,5-dichlorophenyl)-2,3-dihydro-1-benzofuran--yl]methyl}amine,
(±)-{[7-(2,4,6-trichlorophenyl)-2,3-dihydro-1-benzofuran-2-yl]methyl}amine,
(±)-{[7-(4-chloro-2-methylphenyl)-2,3-dihydro-1-benzofuran-2-yl]methyl}amine,
(±)-{[7-(5-chloro-2-methylphenyl)-2,3-dihydro-1-benzofuran-2-yl]methyl}amine,
(±)-{[7-(5-chloro-2-methoxyphenyl)-2,3-dihydro-1-benzofuran-2-yl]methyl}amine,
(±)-[(7-pyridin-3-yl-2,3-dihydro-1-benzofuran-2-yl)methyl]amine,
(+)-{[7-pyridin-3-yl-2,3-dihydro-1-benzofuran-2-yl]methyl}amine,
(−)-{[7-pyridin-3-yl-2,3-dihydro-1-benzofuran-2-yl]methyl}amine,
(±)-{[5-fluoro-7-(2-methoxyphenyl)-2,3-dihydro-1-benzofuran-2-yl]methyl}amine,
(±)-{[5-fluoro-7-(3-fluorophenyl)-2,3-dihydro-1-benzofuran-2-yl]methyl}amine,
(±)-{[5-fluoro-7-(3-methoxyphenyl)-2,3-dihydro-1-benzofuran-2-yl]methyl}amine,
(±)-{[5-fluoro-7-(3-methylphenyl)-2,3-dihydro-1-benzofuran-2-yl]methyl}amine,
(±)-{[5-fluoro-7-(4-fluorophenyl)-2,3-dihydro-1-benzofuran-2-yl]methyl}amine,
(±)-{[5-fluoro-7-(4-chlorophenyl)-2,3-dihydro-1-benzofuran-2-yl]methyl}amine,
(±)-{[5-fluoro-7-(4-methylphenyl)-2,3-dihydro-1-benzofuran-2-yl]methyl}amine,
(±)-{[5-fluoro-7-(4-methoxyphenyl)-2,3-dihydro-1-benzofuran-2-yl]methyl}amine,
(±)-[(5-fluoro-7-thien-3-yl-2,3-dihydro-1-benzofuran-2-yl)methyl]amine,
(±)-{[5-fluoro-7-(3-furyl)-2,3-dihydro-1-benzofuran-2-yl]methyl}amine,
(±)-[(5-fluoro-7-pyridin-2-yl-2,3-dihydro-1-benzofuran-2-yl)methyl]amine,
(±)-[(5-fluoro-7-pyridin-3-yl-2,3-dihydro-1-benzofuran-2-yl)methyl]amine,
(−)-[(5-fluoro-7-pyridin-3-yl-2,3-dihydro-1-benzofuran-2-yl)methyl]amine,
(+)-{[5-fluoro-7-pyridin-3-yl-2,3-dihydro-1-benzofuran-2-yl]methyl}amine,
(±)-[(5-fluoro-7-pyridin-4-yl-2,3-dihydro-1-benzofuran-2-yl)methyl]amine,
(±)-[(5-fluoro-7-pyrimidin-5-yl-2,3-dihydro-1-benzofuran-2-yl)methyl]amine,
(±)-{[7-(2,3-dichlorophenyl)-5-fluoro-2,3-dihydro-1-benzofuran-2-yl]methyl}amine,
(±)-{[7-(2,3-dimethoxyphenyl)-5-fluoro-2,3-dihydro-1-benzofuran-2-yl]methyl}amine,
(−)-{[7-(2,3-dimethoxyphenyl)-5-fluoro-2,3-dihydro-1-benzofuran-2-yl]methyl}amine,
(+)-{[7-(2,3-dimethoxyphenyl)-5-fluoro-2,3-dihydro-1-benzofuran-2-yl]methyl}amine,
(±)-{[7-(2,4-difluorophenyl)-5-fluoro-2,3-dihydro-1-benzofuran-2-yl]methyl}amine,
(±)-{[7-(2,4-dichlorophenyl)-5-fluoro-2,3-dihydro-1-benzofuran-2-yl]methyl}amine,
(−)-{[5-fluoro-7-(2,4-dichlorophenyl)-2,3-dihydro-1-benzofuran-2-yl]methyl}amine,
(+)-{[5-fluoro-7-(2,4-dichlorophenyl)-2,3-dihydro-1-benzofuran-2-yl]methyl}amine,
(±)-{[7-(2,4-dimethoxyphenyl)-5-fluoro-2,3-dihydro-1-benzofuran-2-yl]methyl}amine,
(±)-{[7-(2,5-difluorophenyl)-5-fluoro-2,3-dihydro-1-benzofuran-2-yl]methyl}amine,
(±)-{[7-(2,5-dichlorophenyl)-5-fluoro-2,3-dihydro-1-benzofuran-2-yl]methyl}amine,
(±)-{[7-(2,5-dimethylphenyl)-5-fluoro-2,3-dihydro-1-benzofuran-2-yl]methyl}amine,
(±)-{[7-(2,5-dimethoxyphenyl)-5-fluoro-2,3-dihydro-1-benzofuran-2-yl]methyl}amine,
(±)-{[5-fluoro-7-(5-methoxy-2-methylphenyl)-2,3-dihydro-1-benzofuran-2-yl]methyl}amine,
(±)-{[5-fluoro-7-(2-methoxy-5-methylphenyl)-2,3-dihydro-1-benzofuran-2-yl]methyl}amine,
(±)-{[7-(2,6-difluorophenyl)-5-fluoro-2,3-dihydro-1-benzofuran-2-yl]methyl}amine,
(±)-{[7-(2,6-dimethylphenyl)-5-fluoro-2,3-dihydro-1-benzofuran-2-yl]methyl}amine,
(+)-{[7-(2,6-dimethylphenyl)-5-fluoro-2,3-dihydro-1-benzofuran-2-yl]methyl}amine,
(−)-{[7-(2,6-dimethylphenyl)-5-fluoro-2,3-dihydro-1-benzofuran-2-yl]methyl}amine,
(±)-{[7-(2,6-dichlorophenyl)-5-fluoro-2,3-dihydro-1-benzofuran-2-yl]methyl}amine,
(±)-N-{[7-(2,6-dichlorophenyl)-5-fluoro-2,3-dihydro-1-benzofuran-2-
yl]methyl}cyclopropanamine,
(±)-1-cyclopropyl-N-{[7-(2,6-dichlorophenyl)-5-fluoro-2,3-dihydro-1-benzofuran-2-
yl]methyl}methanamine,
(±)-N-{[7-(2,6-dichlorophenyl)-5-fluoro-2,3-dihydro-1-benzofuran-2-yl]methyl}ethanamine,
(±)-{[7-(2,6-dichlorophenyl)-5-fluoro-2,3-dihydro-1-benzofuran-2-yl]methyl}dimethylamine,
(±)-{[5-chloro-7-(2-fluorophenyl)-2,3-dihydro-1-benzofuran-2-yl]methyl}amine,
(±)-{[5-chloro-7-(2-methoxyphenyl)-2,3-dihydro-1-benzofuran-2-yl]methyl}amine,
(±)-{[5-chloro-7-(3-furyl)-2,3-dihydro-1-benzofuran-2-yl]methyl}amine,
(±)-{[5-chloro-7-(2,3-difluorophenyl)-2,3-dihydro-1-benzofuran-2-yl]methyl}amine,
(−)-{[5-chloro-7-(2,3-difluorophenyl)-2,3-dihydro-1-benzofuran-2-yl]methyl}amine,
(+)-{[5-chloro-7-(2,3-difluorophenyl)-2,3-dihydro-1-benzofuran-2-yl]methyl}amine,
(±)-{[5-chloro-7-(2,3-dichlorophenyl)-2,3-dihydro-1-benzofuran-2-yl]methyl}amine,
(±)-{[5-chloro-7-(2,3-dimethylphenyl)-2,3-dihydro-1-benzofuran-2-yl]methyl}amine,
(±)-{[5-chloro-7-(2,3-dimethoxyphenyl)-2,3-dihydro-1-benzofuran-2-yl]methyl}amine,
(±)-{[5-chloro-7-(2,4-difluorophenyl)-2,3-dihydro-1-benzofuran-2-yl]methyl}amine,
(±)-{[5-chloro-7-(2,4-dichlorophenyl)-2,3-dihydro-1-benzofuran-2-yl]methyl}amine,
(−)-{[5-chloro-7-(2,4-dichlorophenyl)-2,3-dihydro-1-benzofuran-2-yl]methyl}amine,
(+)-{[5-chloro-7-(2,4-dichlorophenyl)-2,3-dihydro-1-benzofuran-2-yl]methyl}amine,
(±)-{[5-chloro-7-(2,4-dimethoxyphenyl)-2,3-dihydro-1-benzofuran-2-yl]methyl}amine,
(±)-{[5-chloro-7-(2,5-difluorophenyl)-2,3-dihydro-1-benzofuran-2-yl]methyl}amine,
(±)-{[5-chloro-7-(2,5-dichlorophenyl)-2,3-dihydro-1-benzofuran-2-yl]methyl}amine,
(±)-{[5-chloro-7-(5-chloro-2-methoxyphenyl)-2,3-dihydro-1-benzofuran-2-yl]methyl}amine,
(±)-{[5-chloro-7-(3,4-difluorophenyl)-2,3-dihydro-1-benzofuran-2-yl]methyl}amine,
(±)-{[5-chloro-7-(3-chloro-4-fluorophenyl)-2,3-dihydro-1-benzofuran-2-yl]methyl}amine,
(±)-{[5-chloro-7-(2,6-dimethylphenyl)-2,3-dihydro-1-benzofuran-2-yl]methyl}amine,
(−)-{[5-chloro-7-(2,6-dimethylphenyl)-2,3-dihydro-1-benzofuran-2-yl]methyl}amine,
(+)-{[5-chloro-7-(2,6-dimethylphenyl)-2,3-dihydro-1-benzofuran-2-yl]methyl}amine,
(±)-{[5-chloro-7-(2,6-dichlorophenyl)-2,3-dihydro-1-benzofuran-2-yl]methyl}amine,
(+)-{[5-chloro-7-(2,6-dichlorophenyl)-2,3-dihydro-1-benzofuran-2-yl]methyl}amine,
(−)-{[5-chloro-7-(2,6-dichlorophenyl)-2,3-dihydro-1-benzofuran-2-yl]methyl}amine,
(±)-{[(5-chloro-7-pyridin-3-yl-2,3-dihydro-1-benzofuran-2-yl)methyl]amine,
(±)-N-{[5-chloro-7-(2,6-dimethylphenyl)-2,3-dihydro-1-benzofuran-2-
yl]methyl}cyclopropanamine,
(±)-N-{[5-chloro-7-(2,6-dimethylphenyl)-2,3-dihydro-1-benzofuran-2-
yl]methyl}(cyclopropylmethyl)amine,
(±)-N-{[5-chloro-7-(2,6-dimethylphenyl)-2,3-dihydro-1-benzofuran-2-yl]methyl}ethanamine,
(±)-{[(5-methyl-7-phenyl-2,3-dihydro-1-benzofuran-2-yl)methyl]amine,
(±)-{[7-(2-methylphenyl)-5-methyl-2,3-dihydro-1-benzofuran-2-yl]methyl}amine,
(±)-{[7-(2-fluorophenyl)-5-methyl-2,3-dihydro-1-benzofuran-2-yl]methyl}amine,
(±)-{[7-(2-methoxyphenyl)-5-methyl-2,3-dihydro-1-benzofuran-2-yl]methyl}amine,
(±)-{[7-(2-chlorophenyl)-5-methyl-2,3-dihydro-1-benzofuran-2-yl]methyl}amine,
(±)-({5-methyl-7-[2-(trifluoromethyl)phenyl]-2,3-dihydro-1-benzofuran-2-yl}methyl)amine,
(±)-{[7-(3-chlorophenyl)-5-methyl-2,3-dihydro-1-benzofuran-2-yl]methyl}amine,
(±)-{[7-(3-methylphenyl)-5-methyl-2,3-dihydro-1-benzofuran-2-yl]methyl}amine,
(±)-{[7-(4-methylphenyl)-5-methyl-2,3-dihydro-1-benzofuran-2-yl]methyl}amine,
(±)-{[7-(4-fluorophenyl)-5-methyl-2,3-dihydro-1-benzofuran-2-yl]methyl}amine,
(±)-{[7-(4-chlorophenyl)-5-methyl-2,3-dihydro-1-benzofuran-2-yl]methyl}amine,
(±)-{[7-(4-methoxyphenyl)-5-methyl-2,3-dihydro-1-benzofuran-2-yl]methyl}amine,
(±)-{[7-(2,3-dimethoxyphenyl)-5-methyl-2,3-dihydro-1-benzofuran-2-yl]methyl}amine,
(−)-{[7-(2,3-dimethoxyphenyl)-5-methyl-2,3-dihydro-1-benzofuran-2-yl]methyl}amine,
(+)-{[7-(2,3-dimethoxyphenyl)-5-methyl-2,3-dihydro-1-benzofuran-2-yl]methyl}amine,
(±)-{[7-(2,4-dichlorophenyl)-5-methyl-2,3-dihydro-1-benzofuran-2-yl]methyl}amine,
(−)-{[7-(2,4-dichlorophenyl)-5-methyl-2,3-dihydro-1-benzofuran-2-yl]methyl}amine,
(+)-{[7-(2,4-dichlorophenyl)-5-methyl-2,3-dihydro-1-benzofuran-2-yl]methyl}amine,
(±)-{[7-(2,5-dichlorophenyl)-5-methyl-2,3-dihydro-1-benzofuran-2-yl]methyl}amine,
(±)-{[7-(2,6-dimethylphenyl)-5-methyl-2,3-dihydro-1-benzofuran-2-yl]methyl}amine,
(±)-{[7-(2,6-dichlorophenyl)-5-methyl-2,3-dihydro-1-benzofuran-2-yl]methyl}amine,
(−)-{[7-(2,6-dichlorophenyl)-5-methyl-2,3-dihydro-1-benzofuran-2-yl]methyl}amine,
(+)-{[7-(2,6-dichlorophenyl)-5-methyl-2,3-dihydro-1-benzofuran-2-yl]methyl}amine,
(±)-{[5-ethyl-7-(2-methylphenyl)-2,3-dihydro-1-benzofuran-2-yl]methyl}amine,
(±)-{[5-ethyl-7-(2-chlorophenyl)-2,3-dihydro-1-benzofuran-2-yl]methyl}amine,
(±)-{[5-(trifluoromethyl)-7-(2-fluorophenyl)-2,3-dihydro-1-benzofuran-2-yl]methyl}amine,
(±)-{[5-(trifluoromethyl)-7-(2-methylphenyl)-2,3-dihydro-1-benzofuran-2-yl]methyl}amine,
(±)-{[5-(trifluoromethyl)-7-(2-chlorophenyl)-2,3-dihydro-1-benzofuran-2-yl]methyl}amine,
(±)-{[5-(trifluoromethyl)-7-(2-methoxyphenyl)-2,3-dihydro-1-benzofuran-2-yl]methyl}amine,
(±)-{[5-(trifluoromethyl)-7-(2-(trifluoromethyl)phenyl)-2,3-dihydro-1-benzofuran-2-
yl]methyl}amine,
(±)-{[5-(trifluoromethyl)-7-(3-methylphenyl)-2,3-dihydro-1-benzofuran-2-yl]methyl}amine,
(±)-{[5-(trifluoromethyl)-7-(3-fluorophenyl)-2,3-dihydro-1-benzofuran-2-yl]methyl}amine,
(±)-{[5-(trifluoromethyl)-7-(3-chlorophenyl)-2,3-dihydro-1-benzofuran-2-yl]methyl}amine,
(±)-{[5-(trifluoromethyl)-7-(3-methoxyphenyl)-2,3-dihydro-1-benzofuran-2-yl]methyl}amine,
(±)-{[5-(trifluoromethyl)-7-(3-(trifluoromethyl)phenyl)-2,3-dihydro-1-benzofuran-2-
yl]methyl}amine,
(±)-{[5-(trifluoromethyl)-7-(4-methylphenyl)-2,3-dihydro-1-benzofuran-2-yl]methyl}amine,
(±)-{[5-(trifluoromethyl)-7-(4-fluorophenyl)-2,3-dihydro-1-benzofuran-2-yl]methyl}amine,
(±)-{[5-(trifluoromethyl)-7-(4-chlorophenyl)-2,3-dihydro-1-benzofuran-2-yl]methyl}amine,
(±)-{[5-(trifluoromethyl)-7-(4-methoxyphenyl)-2,3-dihydro-1-benzofuran-2-yl]methyl}amine,
(±)-{[5-(trifluoromethyl)-7-(4-(trifluoromethyl)phenyl)-2,3-dihydro-1-benzofuran-2-
yl]methyl}amine,
(±)-{[7-(2,3-dimethylphenyl)-5-(trifluoromethyl)-2,3-dihydro-1-benzofuran-2-yl]methyl}amine,
(±)-{[7-(2,3-difluorophenyl)-5-(trifluoromethyl)-2,3-dihydro-1-benzofuran-2-yl]methyl}amine,
(±)-{[7-(2,3-dichlorophenyl)-5-(trifluoromethyl)-2,3-dihydro-1-benzofuran-2-yl]methyl}amine,
(±)-{[7-(2,3-dimethoxyphenyl)-5-(trifluoromethyl)-2,3-dihydro-1-benzofuran-2-
yl]methyl}amine,
(±)-{[7-(2,4-difluorophenyl)-5-(trifluoromethyl)-2,3-dihydro-1-benzofuran-2-yl]methyl}amine,
(±)-{[7-(2,4-dimethoxyphenyl)-5-(trifluoromethyl)-2,3-dihydro-1-benzofuran-2-
yl]methyl}amine,
(±)-{[7-(3,4-difluorophenyl)-5-(trifluoromethyl)-2,3-dihydro-1-benzofuran-2-yl]methyl}amine,
(±)-{[7-(3-chloro-4-fluorophenyl)-5-(trifluoromethyl)-2,3-dihydro-1-benzofuran-2-
yl]methyl}amine,
(±)-{[7-(2,5-difluorophenyl)-5-(trifluoromethyl)-2,3-dihydro-1-benzofuran-2-yl]methyl}amine,
(±)-{[7-(2,5-dichlorophenyl)-5-(trifluoromethyl)-2,3-dihydro-1-benzofuran-2-yl]methyl}amine,
(±)-{[7-(2,6-dimethylphenyl)-5-(trifluoromethyl)-2,3-dihydro-1-benzofuran-2-yl]methyl}amine,
(±)-4-[2-(aminomethyl)-5-(trifluoromethyl)-2,3-dihydro-1-benzofuran-7-yl]benzonitrile
(±)-{[7-(3-furyl)-5-(trifluoromethyl)-2,3-dihydro-1-benzofuran-2-yl]methyl}amine,
(±)-{[7-thien-3-yl-5-(trifluoromethyl)-2,3-dihydro-1-benzofuran-2-yl]methyl}amine,
(±)-{[7-pyridin-3-yl-5-(trifluoromethyl)-2,3-dihydro-1-benzofuran-2-yl]methyl}amine,
(±)-{[7-(2-fluorophenyl)-5-methoxy-2,3-dihydro-1-benzofuran-2-yl]methyl}amine,
(±)-{[7-(2-chlorophenyl)-5-methoxy-2,3-dihydro-1-benzofuran-2-yl]methyl}amine,
(±)-{[7-(2-methylphenyl)-5-methoxy-2,3-dihydro-1-benzofuran-2-yl]methyl}amine,
(±)-{[7-(2-methoxyphenyl)-5-methoxy-2,3-dihydro-1-benzofuran-2-yl]methyl}amine,
(±)-{[5-methoxy-7-(3-thienyl)-2,3-dihydro-1-benzofuran-2-yl]methyl}amine,
(±)-{[7-(2,3-difluorophenyl)-5-methoxy-2,3-dihydro-1-benzofuran-2-yl]methyl}amine,
(±)-{[7-(2,3-dichlorophenyl)-5-methoxy-2,3-dihydro-1-benzofuran-2-yl]methyl}amine,
(±)-{[7-(2,3-dimethylphenyl)-5-methoxy-2,3-dihydro-1-benzofuran-2-yl]methyl}amine,
(±)-{[7-(2,4-difluorophenyl)-5-methoxy-2,3-dihydro-1-benzofuran-2-yl]methyl}amine,
(±)-{[7-(2,4-dichlorophenyl)-5-methoxy-2,3-dihydro-1-benzofuran-2-yl]methyl}amine,
(±)-{[7-(2,5-difluorophenyl)-5-methoxy-2,3-dihydro-1-benzofuran-2-yl]methyl}amine,
(±)-{[7-(2,5-dichlorophenyl)-5-methoxy-2,3-dihydro-1-benzofuran-2-yl]methyl}amine,
(+)-{[7-(2,5-dichlorophenyl)-5-methoxy-2,3-dihydro-1-benzofuran-2-yl]methyl}amine,
(−)-{[7-(2,5-dichlorophenyl)-5-methoxy-2,3-dihydro-1-benzofuran-2-yl]methyl}amine,
(±)-{[7-(2,5-dimethylphenyl)-5-methoxy-2,3-dihydro-1-benzofuran-2-yl]methyl}amine,
(±)-{[7-(2,5-dimethoxylphenyl)-5-methoxy-2,3-dihydro-1-benzofuran-2-yl]methyl}amine,
(±)-{[7-(5-chloro-2-methoxyphenyl)-5-methoxy-2,3-dihydro-1-benzofuran-2-yl]methyl}amine,
(±)-{[7-(3-chloro-4-fluorophenyl)-5-methoxy-2,3-dihydro-1-benzofuran-2-yl]methyl}amine,
(±)-{[7-(2,6-dimethylphenyl)-5-methoxy-2,3-dihydro-1-benzofuran-2-yl]methyl}amine,
(±)-[(N-methyl-1-[7-(2-methoxyphenyl)-2,3-dihydro-1-benzofuran-2-yl]methanamine,
(±)-[(N-methyl-1-[7-(3-methylphenyl)-2,3-dihydro-1-benzofuran-2-yl]methanamine,
(±)-[(N-methyl-1-[7-(3-fluorophenyl)-2,3-dihydro-1-benzofuran-2-yl]methanamine,
(±)-[(N-methyl-1-[7-(3-chlorophenyl)-2,3-dihydro-1-benzofuran-2-yl]methanamine,
(±)-[(N-methyl-1-[7-(3-methoxyphenyl)-2,3-dihydro-1-benzofuran-2-yl]methanamine,
(±)-[(N-methyl-1-[7-(4-methylphenyl)-2,3-dihydro-1-benzofuran-2-yl]methanamine,
(±)-[(N-methyl-1-[7-(4-fluorophenyl)-2,3-dihydro-1-benzofuran-2-yl]methanamine,
(±)-[(N-methyl-1-[7-(4-chlorophenyl)-2,3-dihydro-1-benzofuran-2-yl]methanamine,
(±)-[(N-methyl-1-[7-(4-methoxyphenyl)-2,3-dihydro-1-benzofuran-2-yl]methanamine,
(±)-[(N-methyl-1-[7-(2,3-dimethylphenyl)-2,3-dihydro-1-benzofuran-2-yl]methanamine,
(±)-[(N-methyl-1-[7-(2,3-dimethoxyphenyl)-2,3-dihydro-1-benzofuran-2-yl]methanamine,
(±)-[(N-methyl-1-[7-(2,4-difluorophenyl)-2,3-dihydro-1-benzofuran-2-yl]methanamine,
(±)-[(N-methyl-1-[7-(2,4-dichlorophenyl)-2,3-dihydro-1-benzofuran-2-yl]methanamine,
(±)-[(N-methyl-1-[7-(2,4-dimethoxyphenyl)-2,3-dihydro-1-benzofuran-2-yl]methanamine,
(±)-[(N-methyl-1-[7-(2,5-dimethylphenyl)-2,3-dihydro-1-benzofuran-2-yl]methanamine,
(±)-[(N-methyl-1-[7-(2,5-difluorophenyl)-2,3-dihydro-1-benzofuran-2-yl]methanamine,
(±)-[(N-methyl-1-[7-(2,5-dichlorophenyl)-2,3-dihydro-1-benzofuran-2-yl]methanamine,
(±)-[(N-methyl-1-[7-(5-chloro-2-methoxyphenyl)-2,3-dihydro-1-benzofuran-2-yl]methanamine,
(±)-[(N-methyl-1-[7-(5-chloro-2-methylphenyl)-2,3-dihydro-1-benzofuran-2-yl]methanamine,
(±)-[(N-methyl-1-[7-(2,6-dimethylphenyl)-2,3-dihydro-1-benzofuran-2-yl]methanamine,
(±)-[(N-methyl-1-[7-(2,6-dichlorophenyl)-2,3-dihydro-1-benzofuran-2-yl]methanamine,
(−)-{[7-(2,6-dichlorophenyl)-2,3-dihydro-1-benzofuran-2-yl]methyl}methylamine,
(+)-{[7-(2,6-dichlorophenyl)-2,3-dihydro-1-benzofuran-2-yl]methyl}methylamine,
(±)-N-methyl-1-(7-pyridin-3-yl-2,3-dihydro-1-benzofuran-2-yl)methanamine,
(±)-[(N-methyl-1-[7-(2,3-difluorophenyl)-2,3-dihydro-1-benzofuran-2-yl]methanamine,
(±)-{[5-fluoro-7-(2-fluorophenyl)-2,3-dihydro-1-benzofuran-2-yl]methyl}methylamine,
(±)-{[5-fluoro-7-(2-chlorophenyl)-2,3-dihydro-1-benzofuran-2-yl]methyl}methylamine,
(±)-{[5-fluoro-7-(2-methylphenyl)-2,3-dihydro-1-benzofuran-2-yl]methyl}methylamine,
(−)-{[5-fluoro-7-(2-methylphenyl)-2,3-dihydro-1-benzofuran-2-yl]methyl}methylamine,
(+)-{[5-fluoro-7-(2-methylphenyl)-2,3-dihydro-1-benzofuran-2-yl]methyl}methylamine,
(±)-{[5-fluoro-7-(2-methoxyphenyl)-2,3-dihydro-1-benzofuran-2-yl]methyl}methylamine,
(±)-{[7-(2,3-dimethoxyphenyl)-5-fluoro-2,3-dihydro-1-benzofuran-2-yl]methyl}methylamine,
(±)-[7-(2,4-dichlorophenyl)-5-fluoro-2,3-dihydro-1-benzofuran-2-yl]methyl}methylamine,
(−)-[7-(2,4-dichlorophenyl)-5-fluoro-2,3-dihydro-1-benzofuran-2-yl]methyl}methylamine,
(+)-[7-(2,4-dichlorophenyl)-5-fluoro-2,3-dihydro-1-benzofuran-2-yl]methyl}methylamine,
(±)-{[7-(2,5-difluorophenyl)-5-fluoro-2,3-dihydro-1-benzofuran-2-yl]methyl}methylamine,
(±)-{[7-(2,5-dichlorophenyl)-5-fluoro-2,3-dihydro-1-benzofuran-2-yl]methyl}methylamine,
(+)-{[5-fluoro-7-(2,5-dichlorophenyl)-2,3-dihydro-1-benzofuran-2-yl]methyl}methylamine,
(−)-{[5-fluoro-7-(2,5-dichlorophenyl)-2,3-dihydro-1-benzofuran-2-yl]methyl}methylamine,
(±)-{[7-(2,5-dimethylphenyl)-5-fluoro-2,3-dihydro-1-benzofuran-2-yl]methyl}methylamine,
(±)-{[7-(2,6-dimethylphenyl)-5-fluoro-2,3-dihydro-1-benzofuran-2-yl]methyl}methylamine,
(±)-{[7-(2,6-dichlorophenyl)-5-fluoro-2,3-dihydro-1-benzofuran-2-yl]methyl}methylamine,
(±)-{[5-fluoro-7-(5-methoxy-2-methylphenyl)-2,3-dihydro-1-benzofuran-2-
yl]methyl}methylamine,
(±)-{[5-fluoro-7-(2-methoxy-5-methylphenyl)-2,3-dihydro-1-benzofuran-2-
yl]methyl}methylamine,
(±)-{[7-(5-chloro-2-methoxyphenyl)-5-fluoro-2,3-dihydro-1-benzofuran-2-
yl]methyl}methylamine,
(±)-[(5-fluoro-7-pyridin-3-yl-2,3-dihydro-1-benzofuran-2-yl)methyl]methylamine,
(±)-[(5-chloro-7-(2-methoxyphenyl)-2,3-dihydro-1-benzofuran-2-yl]methyl}methylamine,
(±)-{[5-chloro-7-(2,6-dimethylphenyl)-2,3-dihydro-1-benzofuran-2-yl]methyl}methylamine,
(±)-[(5-chloro-7-(2-fluorophenyl)-2,3-dihydro-1-benzofuran-2-yl]methyl}methylamine,
(±)-{[5-chloro-7-(2,3-difluorophenyl)-2,3-dihydro-1-benzofuran-2-yl]methyl}methylamine,
(±)-{[5-chloro-7-(2,3-dichlorophenyl)-2,3-dihydro-1-benzofuran-2-yl]methyl}methylamine,
(±)-{[5-chloro-7-(2,3-dimethylphenyl)-2,3-dihydro-1-benzofuran-2-yl]methyl}methylamine,
(±)-{[5-chloro-7-(2,3-dimethoxyphenyl)-2,3-dihydro-1-benzofuran-2-yl]methyl}methylamine,
(±)-{[5-chloro-7-(2,4-difluorophenyl)-2,3-dihydro-1-benzofuran-2-yl]methyl}methylamine,
(±)-{[5-chloro-7-(2,4-dichlorophenyl)-2,3-dihydro-1-benzofuran-2-yl]methyl}methylamine,
(±)-{[5-chloro-7-(2,4-dimethoxyphenyl)-2,3-dihydro-1-benzofuran-2-yl]methyl}methylamine,
(±)-{[5-chloro-7-(2,5-difluorophenyl)-2,3-dihydro-1-benzofuran-2-yl]methyl}methylamine,
(±)-{[5-chloro-7-(2,5-dichlorophenyl)-2,3-dihydro-1-benzofuran-2-yl]methyl}methylamine,
(±)-{[5-chloro-7-(5-chloro-2-methoxyphenyl)-2,3-dihydro-1-benzofuran-2-
yl]methyl}methylamine,
(±)-{[5-chloro-7-(3,4-difluorophenyl)-2,3-dihydro-1-benzofuran-2-yl]methyl}methylamine,
(±)-[5-chloro-7-(3-chloro-4-fluorophenyl)-2,3-dihydro-1-benzofuran-2-yl]methyl}methylamine,
(±)-{[7-(2-fluorophenyl)-5-methyl-2,3-dihydro-1-benzofuran-2-yl]methyl}methylamine,
(±)-{[7-(2-chlorophenyl)-5-methyl-2,3-dihydro-1-benzofuran-2-yl]methyl}methylamine,
(±)-{[7-(2-methoxyphenyl)-5-methyl-2,3-dihydro-1-benzofuran-2-yl]methyl}methylamine,
(±)-{[7-(3-methylphenyl)-5-methyl-2,3-dihydro-1-benzofuran-2-yl]methyl}methylamine,
(±)-{[7-(3-chlorophenyl)-5-methyl-2,3-dihydro-1-benzofuran-2-yl]methyl}methylamine,
(±)-{[7-(4-methylphenyl)-5-methyl-2,3-dihydro-1-benzofuran-2-yl]methyl}methylamine,
(±)-{[7-(4-chlorophenyl)-5-methyl-2,3-dihydro-1-benzofuran-2-yl]methyl}methylamine,
(±)-{[7-(4-fluorophenyl)-5-methyl-2,3-dihydro-1-benzofuran-2-yl]methyl}methylamine,
(±)-{[7-(4-methoxyphenyl)-5-methyl-2,3-dihydro-1-benzofuran-2-yl]methyl}methylamine,
(±)-{[7-(2,3-dimethoxyphenyl)-5-methyl-2,3-dihydro-1-benzofuran-2-yl]methyl}methylamine,
(±)-{[7-(2,4-dichlorophenyl)-5-methyl-2,3-dihydro-1-benzofuran-2-yl]methyl}methylamine,
(±)-{[7-(2,5-dichlorophenyl)-5-methyl-2,3-dihydro-1-benzofuran-2-yl]methyl}methylamine,
(+)-{[7-(2,5-dichlorophenyl)-5-methyl-2,3-dihydro-1-benzofuran-2-yl]methyl}methylamine,
(−)-{[7-(2,5-dichlorophenyl)-5-methyl-2,3-dihydro-1-benzofuran-2-yl]methyl}methylamine,
(±)-{[7-(2,6-dimethylphenyl)-5-methyl-2,3-dihydro-1-benzofuran-2-yl]methyl}methylamine,
(±)-{[7-(2,6-dichlorophenyl)-5-methyl-2,3-dihydro-1-benzofuran-2-yl]methyl}methylamine,
(±)-{[7-(2-fluorophenyl)-5-methoxy-2,3-dihydro-1-benzofuran-2-yl]methyl}methylamine,
(±)-{[7-(2-chlorophenyl)-5-methoxy-2,3-dihydro-1-benzofuran-2-yl]methyl}methylamine,
(±)-{[7-(2-methylphenyl)-5-methoxy-2,3-dihydro-1-benzofuran-2-yl]methyl}methylamine,
(±)-{[7-(2,3-difluorophenyl)-5-methoxy-2,3-dihydro-1-benzofuran-2-yl]methyl}methylamine,
(±)-{[7-(2,3-dichlorophenyl)-5-methoxy-2,3-dihydro-1-benzofuran-2-yl]methyl}methylamine,
(±)-{[7-(2,3-dimethylphenyl)-5-methoxy-2,3-dihydro-1-benzofuran-2-yl]methyl}methylamine,
(±)-{[7-(2,4-difluorophenyl)-5-methoxy-2,3-dihydro-1-benzofuran-2-yl]methyl}methylamine,
(±)-{[7-(2,5-difluorophenyl)-5-methoxy-2,3-dihydro-1-benzofuran-2-yl]methyl}methylamine,
(±)-{[7-(2,5-dichlorophenyl)-5-methoxy-2,3-dihydro-1-benzofuran-2-yl]methyl}methylamine,
(±)-{[7-(2,5-dimethylphenyl)-5-methoxy-2,3-dihydro-1-benzofuran-2-yl]methyl}methylamine,
(±)-{[7-(2,5-dimethoxyphenyl)-5-methoxy-2,3-dihydro-1-benzofuran-2-yl]methyl}methylamine,
(±)-{[7-(5-chloro-2-methoxyphenyl)-5-methoxy-2,3-dihydro-1-benzofuran-2-
yl]methyl}methylamine,
(±)-{[7-(2,6-dimethylphenyl)-5-methoxy-2,3-dihydro-1-benzofuran-2-yl]methyl}methylamine,
(±)-N-methyl-1-[7-(2,3-difluorophenyl)-5-(trifluoromethyl)-2,3-dihydro-1-benzofuran-2-
yl]methanamine,
(±)-N-methyl-1-[7-(3,4-difluorophenyl)-5-(trifluoromethyl)-2,3-dihydro-1-benzofuran-2-
yl]methanamine,
(±)-N-methyl-1-[7-(2,5-difluorophenyl)-5-(trifluoromethyl)-2,3-dihydro-1-benzofuran-2-
yl]methanamine,
(±)-N-methyl-1-[7-(2,3-dichlorophenyl)-5-(trifluoromethyl)-2,3-dihydro-1-benzofuran-2-
yl]methanamine,
(±)-{[7-(3-chloro-4-fluorophenyl)-5-(trifluoromethyl)-2,3-dihydro-1-benzofuran-2-
yl]methyl}methylamine,
(±)-N-methyl-1-[7-(2,4-dimethoxyphenyl)-5-(trifluoromethyl)-2,3-dihydro-1-benzofuran-2-
yl]methanamine,
(+){[7-(2,6-dichlorophenyl)-5-fluoro-2,3-dihydro-1-benzofuran-2-yl]methyl}methylamine,
(−){[7-(2,6-dichlorophenyl)-5-fluoro-2,3-dihydro-1-benzofuran-2-yl]methyl}methylamine,
(R)-[7-(2-chloro-phenyl)-(5-Fluoro-2,3-dihydro-benzofuran-2-ylmethyl)methyl-amine,
(R)-[7-(2,6-dichloro-phenyl)-5-Fluoro-2,3-dihydro-benzofuran-2-ylmethyl]ethylamine,
(R)-[7-(2,6-dichloro-phenyl)-5-Fluoro-2,3-dihydro-benzofuran-2-ylmethyl]dimethylamine,
{[(2R)-7-(5-chloro-2-methylphenyl)-5-fluoro-2,3-dihydro-1-benzofuran-2-yl]methyl}amine,
{[(2R)-7-(4-chloro-2-methylphenyl)-5-fluoro-2,3-dihydro-1-benzofuran-2-yl]methyl}amine,
(−)-{[7-(2,6-dichlorophenyl)-5-fluoro-2,3-dihydro-1-benzofuran-2-yl]methyl}amine,
(+)-{[7-(2,6-dichlorophenyl)-5-fluoro-2,3-dihydro-1-benzofuran-2-yl]methyl}amine,
(±)-{2-[6-chloro-7-(2-chlorophenyl)-2,3-dihydro-1-benzofuran-2-yl]ethyl}amine,
(±)-{2-[7-(2,6-dichlorophenyl)-5-fluoro-2,3-dihydro-1-benzofuran-2-yl]ethyl}amine,
(±)-{2-[7-(2-methoxyphenyl)-5-methoxy-2,3-dihydro-1-benzofuran-2-yl]ethyl}amine,
(±)-{N-methyl-1-[(7-(2,4,6-trichlorophenyl)-2,3-dihydro-1-benzofuran-2-yl]methanamine,
(+)-{N-methyl-1-[(7-(2,4,6-trichlorophenyl)-2,3-dihydro-1-benzofuran-2-yl]methanamine,
(−)-{N-methyl-1-[(7-(2,4,6-trichlorophenyl)-2,3-dihydro-1-benzofuran-2-yl]methanamine,
(+)-{[7-(2,6-dimethylphenyl)-5-fluoro-2,3-dihydro-1-benzofuran-2-yl]methyl}methylamine,
(−)-{[7-(2,6-dimethylphenyl)-5-fluoro-2,3-dihydro-1-benzofuran-2-yl]methyl}methylamine,
(−)-{[7-(2,6-dimethylphenyl)-5-methoxy-2,3-dihydro-1-benzofuran-2-yl]methyl}methylamine,
(+)-{[7-(2,6-dimethylphenyl)-5-methoxy-2,3-dihydro-1-benzofuran-2-yl]methyl}methylamine,
(+)-{[5-chloro-7-(2,5-dichlorophenyl)-2,3-dihydro-1-benzofuran-2-yl]methyl}methylamine,
(−)-{[5-chloro-7-(2,5-dichlorophenyl)-2,3-dihydro-1-benzofuran-2-yl]methyl}methylamine,
(−)-{[5-chloro-7-(2,6-dimethylphenyl)-2,3-dihydro-1-benzofuran-2-yl]methyl}methylamine,
(+)-{[5-chloro-7-(2,6-dimethylphenyl)-2,3-dihydro-1-benzofuran-2-yl]methyl}methylamine,
(+)-{[7-(2,3-dimethoxyphenyl)-5-methyl-2,3-dihydro-1-benzofuran-2-yl]methyl}methylamine,
(−)-{[7-(2,3-dimethoxyphenyl)-5-methyl-2,3-dihydro-1-benzofuran-2-yl]methyl}methylamine,
(−)-{[7-(2,3-dimethoxyphenyl)-5-fluoro-2,3-dihydro-1-benzofuran-2-yl]methyl}methylamine, or
(+)-{[7-(2,3-dimethoxyphenyl)-5-fluoro-2,3-dihydro-1-benzofuran-2-yl]methyl}methylamine;
or a pharmaceutically acceptable salt thereof.

In certain embodiments, exemplary compounds of formula I are as set forth in Table 1-a, below.

TABLE 1-a
Exemplary Compounds of formula I:

(±)-1-(7-phenyl-2,3-dihydro-1-benzofuran-2-yl)methanamine,
(+)-(1-(7-phenyl-2,3-dihydro-1-benzofuran-2-yl)methanamine,
(−)-1-(7-phenyl-2,3-dihydro-1-benzofuran-2-yl)methanamine,
(±)-1-[7-(2-methylphenyl)-2,3-dihydro-1-benzofuran-2-yl]methanamine,
(+)-1-[7-(2-methylphenyl)-2,3-dihydro-1-benzofuran-2-yl]methanamine,
(−)-1-[7-(2-methylphenyl)-2,3-dihydro-1-benzofuran-2-yl]methanamine,
(±)-1-[7-(2-fluorophenyl)-2,3-dihydro-1-benzofuran-2-yl]methanamine,
(+)-1-[7-(2-fluorophenyl)-2,3-dihydro-1-benzofuran-2-yl]methanamine,
(−)-1-[7-(2-fluorophenyl)-2,3-dihydro-1-benzofuran-2-yl]methanamine,
(±)-1-{7-[2-(trifluoromethyl)phenyl]-2,3-dihydro-1-benzofuran-2-yl}methanamine,
(−)-1-{7-[2-(trifluoromethyl)phenyl]-2,3-dihydro-1-benzofuran-2-yl}methanamine,
(+)-1-{7-[2-(trifluoromethyl)phenyl]-2,3-dihydro-1-benzofuran-2-yl}methanamine,
(±)-1-[7-(2,6-dimethylphenyl)-2,3-dihydro-1-benzofuran-2-yl]methanamine,
(−)-1-[7-(2,6-dimethylphenyl)-2,3-dihydro-1-benzofuran-2-yl]methanamine,
(+)-1-[7-(2,6-dimethylphenyl)-2,3-dihydro-1-benzofuran-2-yl]methanamine,
(±)-1-[7-(2-methoxyphenyl)-2,3-dihydro-1-benzofuran-2-yl]methanamine,
(±)-1-[7-(2-chlorophenyl)-2,3-dihydro-1-benzofuran-2-yl]methanamine,
(±)-1-[7-(2,4-dichlorophenyl)-2,3-dihydro-1-benzofuran-2-yl]methanamine,
(±)-1-[5-chloro-7-(2-chlorophenyl)-2,3-dihydro-1-benzofuran-2-yl]methanamine,
(±)-1-[5-chloro-7-(2-methylphenyl)-2,3-dihydro-1-benzofuran-2-yl]methanamine,
(+)-1-[5-chloro-7-(2-methylphenyl)-2,3-dihydro-1-benzofuran-2-yl]methanamine,
(−)-1-[5-chloro-7-(2-methylphenyl)-2,3-dihydro-1-benzofuran-2-yl]methanamine,
(±)-1-[4-fluoro-7-(2-methylphenyl)-2,3-dihydro-1-benzofuran-2-yl]methanamine,
(±)-1-[7-(2-chlorophenyl)-5-fluoro-2,3-dihydro-1-benzofuran-2-yl]methanamine,
(+)-1-[7-(2-chlorophenyl)-5-fluoro-2,3-dihydro-1-benzofuran-2-yl]methanamine,
(−)-1-[7-(2-chlorophenyl)-5-fluoro-2,3-dihydro-1-benzofuran-2-yl]methanamine,
(±)-1-[5-fluoro-7-(2-methylphenyl)-2,3-dihydro-1-benzofuran-2-yl]methanamine,
(+)-1-[5-fluoro-7-(2-methylphenyl)-2,3-dihydro-1-benzofuran-2-yl]methanamine,
(−)-1-[5-fluoro-7-(2-methylphenyl)-2,3-dihydro-1-benzofuran-2-yl]methanamine,
(±)-1-[5-fluoro-7-(2-fluorophenyl)-2,3-dihydro-1-benzofuran-2-yl]methanamine,
(±)-1-{5-fluoro-7-[2-(trifluoromethyl)phenyl]-2,3-dihydro-1-benzofuran-2-yl}methanamine,
(±)-1-[4,5-difluoro-7-(2-methylphenyl)-2,3-dihydro-1-benzofuran-2-yl]methanamine,
(±)-1-(5-chloro-2-methyl-7-phenyl-2,3-dihydro-1-benzofuran-2-yl)methanamine,
(+)-1-[7-(2-methoxyphenyl)-2,3-dihydro-1-benzofuran-2-yl]methanamine,
(−)-1-[7-(2-chlorophenyl)-2,3-dihydro-1-benzofuran-2-yl]methanamine,
(+)-1-[7-(2-chlorophenyl)-2,3-dihydro-1-benzofuran-2-yl]methanamine,
(±)-1-[7-(2,6-difluorophenyl)-2,3-dihydro-1-benzofuran-2-yl]methanamine,
(±)-1-[7-(2,6-dichlorophenyl)-2,3-dihydro-1-benzofuran-2-yl]methanamine,
(−)-1-[7-(2,6-dichlorophenyl)-2,3-dihydro-1-benzofuran-2-yl]methanamine,
(+)-1-[7-(2,6-dichlorophenyl)-2,3-dihydro-1-benzofuran-2-yl]methanamine,
(±)-1-[7-(2,4-dimethoxyphenyl)-2,3-dihydro-1-benzofuran-2-yl]methanamine,
(−)-1-[7-(2,4-dimethoxyphenyl)-2,3-dihydro-1-benzofuran-2-yl]methanamine,
(+)-1-[7-(2,4-dimethoxyphenyl)-2,3-dihydro-1-benzofuran-2-yl]methanamine,
(±)-1-[7-(2,4-difluorophenyl)-2,3-dihydro-1-benzofuran-2-yl]methanamine,
(+)-1-[7-(2,4-difluorophenyl)-2,3-dihydro-1-benzofuran-2-yl]methanamine,
(−)-1-[7-(2,4-difluorophenyl)-2,3-dihydro-1-benzofuran-2-yl]methanamine,
(−)-1-[7-(2,4-dichlorophenyl)-2,3-dihydro-1-benzofuran-2-yl]methanamine,
(+)-1-[7-(2,4-dichlorophenyl)-2,3-dihydro-1-benzofuran-2-yl]methanamine,
(−)-1-{5-fluoro-7-[2-(trifluoromethyl)phenyl]-2,3-dihydro-1-benzofuran-2-yl}methanamine,
(+)-1-{5-fluoro-7-[2-(trifluoromethyl)phenyl]-2,3-dihydro-1-benzofuran-2-yl}methanamine,
(±)-1-[7-(2,3-dimethylphenyl)-2,3-dihydro-1-benzofuran-2-yl]methanamine,
(±)-{[7-(2,3-dimethoxyphenyl)-2,3-dihydro-1-benzofuran-2-yl]methyl}amine,
(−)-{[(7-(2,3-dimethoxyphenyl)-2,3-dihydro-1-benzofuran-2-yl]methyl}amine,
(+)-{[(7-(2,3-dimethoxyphenyl)-2,3-dihydro-1-benzofuran-2-yl]methyl}amine,
(+)-{[7-(4-chloro-2-methylphenyl)-2,3-dihydro-1-benzofuran-2-yl]methyl}amine,
(−)-{[7-(4-chloro-2-methylphenyl)-2,3-dihydro-1-benzofuran-2-yl]methyl}amine,
(±)-{[7-(2,3-difluorophenyl)-2,3-dihydro-1-benzofuran-2-yl]methyl}amine,
(±)-{[7-(2,5-dimethylphenyl)-2,3-dihydro-1-benzofuran-2-yl]methyl}amine,
(±)-{[7-(2,5-dimethoxyphenyl)-2,3-dihydro-1-benzofuran-2-yl]methyl}amine,
(±)-{[7-(2,5-dichlorophenyl)-2,3-dihydro-1-benzofuran-2-yl]methyl}amine,
(+)-{[7-(2,5-dichlorophenyl)-2,3-dihydro-1-benzofuran-2-yl]methyl}amine,
(−)-{[7-(2,5-dichlorophenyl)-2,3-dihydro-1-benzofuran--yl]methyl}amine,
(±)-{[7-(2,4,6-trichlorophenyl)-2,3-dihydro-1-benzofuran-2-yl]methyl}amine,
(±)-{[7-(4-chloro-2-methylphenyl)-2,3-dihydro-1-benzofuran-2-yl]methyl}amine,
(±)-{[7-(5-chloro-2-methylphenyl)-2,3-dihydro-1-benzofuran-2-yl]methyl}amine,
(±)-{[7-(5-chloro-2-methoxyphenyl)-2,3-dihydro-1-benzofuran-2-yl]methyl}amine,
(±)-{[5-fluoro-7-(2-methoxyphenyl)-2,3-dihydro-1-benzofuran-2-yl]methyl}amine,
(±)-{[7-(2,3-dichlorophenyl)-5-fluoro-2,3-dihydro-1-benzofuran-2-yl]methyl}amine,
(±)-{[7-(2,3-dimethoxyphenyl)-5-fluoro-2,3-dihydro-1-benzofuran-2-yl]methyl}amine,
(−)-{[7-(2,3-dimethoxyphenyl)-5-fluoro-2,3-dihydro-1-benzofuran-2-yl]methyl}amine,
(+)-{[7-(2,3-dimethoxyphenyl)-5-fluoro-2,3-dihydro-1-benzofuran-2-yl]methyl}amine,
(±)-{[7-(2,4-difluorophenyl)-5-fluoro-2,3-dihydro-1-benzofuran-2-yl]methyl}amine,
(±)-{[7-(2,4-dichlorophenyl)-5-fluoro-2,3-dihydro-1-benzofuran-2-yl]methyl}amine,
(−)-{[5-fluoro-7-(2,4-dichlorophenyl)-2,3-dihydro-1-benzofuran-2-yl]methyl}amine,
(+)-{[5-fluoro-7-(2,4-dichlorophenyl)-2,3-dihydro-1-benzofuran-2-yl]methyl}amine,
(±)-{[7-(2,4-dimethoxyphenyl)-5-fluoro-2,3-dihydro-1-benzofuran-2-yl]methyl}amine,
(±)-{[7-(2,5-difluorophenyl)-5-fluoro-2,3-dihydro-1-benzofuran-2-yl]methyl}amine,
(±)-{[7-(2,5-dichlorophenyl)-5-fluoro-2,3-dihydro-1-benzofuran-2-yl]methyl}amine,
(±)-{[7-(2,5-dimethylphenyl)-5-fluoro-2,3-dihydro-1-benzofuran-2-yl]methyl}amine,
(±)-{[7-(2,5-dimethoxyphenyl)-5-fluoro-2,3-dihydro-1-benzofuran-2-yl]methyl}amine,
(±)-{[5-fluoro-7-(5-methoxy-2-methylphenyl)-2,3-dihydro-1-benzofuran-2-yl]methyl}amine,
(±)-{[5-fluoro-7-(2-methoxy-5-methylphenyl)-2,3-dihydro-1-benzofuran-2-yl]methyl}amine,
(±)-{[7-(2,6-difluorophenyl)-5-fluoro-2,3-dihydro-1-benzofuran-2-yl]methyl}amine,
(±)-{[7-(2,6-dimethylphenyl)-5-fluoro-2,3-dihydro-1-benzofuran-2-yl]methyl}amine,
(+)-{[7-(2,6-dimethylphenyl)-5-fluoro-2,3-dihydro-1-benzofuran-2-yl]methyl}amine,
(−)-{[7-(2,6-dimethylphenyl)-5-fluoro-2,3-dihydro-1-benzofuran-2-yl]methyl}amine,
(±)-{[7-(2,6-dichlorophenyl)-5-fluoro-2,3-dihydro-1-benzofuran-2-yl]methyl}amine,
(±)-N-{[7-(2,6-dichlorophenyl)-5-fluoro-2,3-dihydro-1-benzofuran-2-
yl]methyl}cyclopropanamine,
(±)-1-cyclopropyl-N-{[7-(2,6-dichlorophenyl)-5-fluoro-2,3-dihydro-1-benzofuran-2-
yl]methyl}methanamine,
(±)-N-{[7-(2,6-dichlorophenyl)-5-fluoro-2,3-dihydro-1-benzofuran-2-yl]methyl}ethanamine,
(±)-{[7-(2,6-dichlorophenyl)-5-fluoro-2,3-dihydro-1-benzofuran-2-yl]methyl}dimethylamine,
(±)-{[5-chloro-7-(2-fluorophenyl)-2,3-dihydro-1-benzofuran-2-yl]methyl}amine,
(±)-{[5-chloro-7-(2-methoxyphenyl)-2,3-dihydro-1-benzofuran-2-yl]methyl}amine,
(±)-{[5-chloro-7-(2,3-difluorophenyl)-2,3-dihydro-1-benzofuran-2-yl]methyl}amine,
(−)-{[5-chloro-7-(2,3-difluorophenyl)-2,3-dihydro-1-benzofuran-2-yl]methyl}amine,
(+)-{[5-chloro-7-(2,3-difluorophenyl)-2,3-dihydro-1-benzofuran-2-yl]methyl}amine,
(±)-{[5-chloro-7-(2,3-dichlorophenyl)-2,3-dihydro-1-benzofuran-2-yl]methyl}amine,
(±)-{[5-chloro-7-(2,3-dimethylphenyl)-2,3-dihydro-1-benzofuran-2-yl]methyl}amine,
(±)-{[5-chloro-7-(2,3-dimethoxyphenyl)-2,3-dihydro-1-benzofuran-2-yl]methyl}amine,
(±)-{[5-chloro-7-(2,4-difluorophenyl)-2,3-dihydro-1-benzofuran-2-yl]methyl}amine,
(±)-{[5-chloro-7-(2,4-dichlorophenyl)-2,3-dihydro-1-benzofuran-2-yl]methyl}amine,
(−)-{[5-chloro-7-(2,4-dichlorophenyl)-2,3-dihydro-1-benzofuran-2-yl]methyl}amine,
(+)-{[5-chloro-7-(2,4-dichlorophenyl)-2,3-dihydro-1-benzofuran-2-yl]methyl}amine,
(±)-{[5-chloro-7-(2,4-dimethoxyphenyl)-2,3-dihydro-1-benzofuran-2-yl]methyl}amine,
(±)-{[5-chloro-7-(2,5-difluorophenyl)-2,3-dihydro-1-benzofuran-2-yl]methyl}amine,
(±)-{[5-chloro-7-(2,5-dichlorophenyl)-2,3-dihydro-1-benzofuran-2-yl]methyl}amine,
(±)-{[5-chloro-7-(5-chloro-2-methoxyphenyl)-2,3-dihydro-1-benzofuran-2-yl]methyl}amine,
(±)-{[5-chloro-7-(2,6-dimethylphenyl)-2,3-dihydro-1-benzofuran-2-yl]methyl}amine,
(−)-{[5-chloro-7-(2,6-dimethylphenyl)-2,3-dihydro-1-benzofuran-2-yl]methyl}amine,
(+)-{[5-chloro-7-(2,6-dimethylphenyl)-2,3-dihydro-1-benzofuran-2-yl]methyl}amine,
(±)-{[5-chloro-7-(2,6-dichlorophenyl)-2,3-dihydro-1-benzofuran-2-yl]methyl}amine,
(+)-{[5-chloro-7-(2,6-dichlorophenyl)-2,3-dihydro-1-benzofuran-2-yl]methyl}amine,
(−)-{[5-chloro-7-(2,6-dichlorophenyl)-2,3-dihydro-1-benzofuran-2-yl]methyl}amine,
(±)-N-{[5-chloro-7-(2,6-dimethylphenyl)-2,3-dihydro-1-benzofuran-2-
yl]methyl}cyclopropanamine,
(±)-N-{[5-chloro-7-(2,6-dimethylphenyl)-2,3-dihydro-1-benzofuran-2-
yl]methyl}(cyclopropylmethyl)amine,
(±)-N-{[5-chloro-7-(2,6-dimethylphenyl)-2,3-dihydro-1-benzofuran-2-yl]methyl}ethanamine,
(±)-{[7-(2-methylphenyl)-5-methyl-2,3-dihydro-1-benzofuran-2-yl]methyl}amine,
(±)-{[7-(2-fluorophenyl)-5-methyl-2,3-dihydro-1-benzofuran-2-yl]methyl}amine,
(±)-{[7-(2-methoxyphenyl)-5-methyl-2,3-dihydro-1-benzofuran-2-yl]methyl}amine,
(±)-{[7-(2-chlorophenyl)-5-methyl-2,3-dihydro-1-benzofuran-2-yl]methyl}amine,
(±)-({5-methyl-7-[2-(trifluoromethyl)phenyl]-2,3-dihydro-1-benzofuran-2-yl}methyl)amine,
(±)-{[7-(2,3-dimethoxyphenyl)-5-methyl-2,3-dihydro-1-benzofuran-2-yl]methyl}amine,
(−)-{[7-(2,3-dimethoxyphenyl)-5-methyl-2,3-dihydro-1-benzofuran-2-yl]methyl}amine,
(+)-{[7-(2,3-dimethoxyphenyl)-5-methyl-2,3-dihydro-1-benzofuran-2-yl]methyl}amine,
(±)-{[7-(2,4-dichlorophenyl)-5-methyl-2,3-dihydro-1-benzofuran-2-yl]methyl}amine,
(−)-{[7-(2,4-dichlorophenyl)-5-methyl-2,3-dihydro-1-benzofuran-2-yl]methyl}amine,
(+)-{[7-(2,4-dichlorophenyl)-5-methyl-2,3-dihydro-1-benzofuran-2-yl]methyl}amine,
(±)-{[7-(2,5-dichlorophenyl)-5-methyl-2,3-dihydro-1-benzofuran-2-yl]methyl}amine,
(±)-{[7-(2,6-dimethylphenyl)-5-methyl-2,3-dihydro-1-benzofuran-2-yl]methyl}amine,
(±)-{[7-(2,6-dichlorophenyl)-5-methyl-2,3-dihydro-1-benzofuran-2-yl]methyl}amine,
(−)-{[7-(2,6-dichlorophenyl)-5-methyl-2,3-dihydro-1-benzofuran-2-yl]methyl}amine,
(+)-{[7-(2,6-dichlorophenyl)-5-methyl-2,3-dihydro-1-benzofuran-2-yl]methyl}amine,
(±)-{[5-ethyl-7-(2-methylphenyl)-2,3-dihydro-1-benzofuran-2-yl]methyl}amine,
(±)-{[5-ethyl-7-(2-chlorophenyl)-2,3-dihydro-1-benzofuran-2-yl]methyl}amine,
(±)-{[5-(trifluoromethyl)-7-(2-fluorophenyl)-2,3-dihydro-1-benzofuran-2-yl]methyl}amine,
(±)-{[5-(trifluoromethyl)-7-(2-methylphenyl)-2,3-dihydro-1-benzofuran-2-yl]methyl}amine,
(±)-{[5-(trifluoromethyl)-7-(2-chlorophenyl)-2,3-dihydro-1-benzofuran-2-yl]methyl}amine,
(±)-{[5-(trifluoromethyl)-7-(2-methoxyphenyl)-2,3-dihydro-1-benzofuran-2-yl]methyl}amine,
(±)-{[5-(trifluoromethyl)-7-(2-(trifluoromethyl)phenyl)-2,3-dihydro-1-benzofuran-2-
yl]methyl}amine,
(±)-{[7-(2,3-dimethylphenyl)-5-(trifluoromethyl)-2,3-dihydro-1-benzofuran-2-yl]methyl}amine,
(±)-{[7-(2,3-difluorophenyl)-5-(trifluoromethyl)-2,3-dihydro-1-benzofuran-2-yl]methyl}amine,
(±)-{[7-(2,3-dichlorophenyl)-5-(trifluoromethyl)-2,3-dihydro-1-benzofuran-2-yl]methyl}amine,
(±)-{[7-(2,3-dimethoxyphenyl)-5-(trifluoromethyl)-2,3-dihydro-1-benzofuran-2-
yl]methyl}amine,
(±)-{[7-(2,4-difluorophenyl)-5-(trifluoromethyl)-2,3-dihydro-1-benzofuran-2-yl]methyl}amine,
(±)-{[7-(2,4-dimethoxyphenyl)-5-(trifluoromethyl)-2,3-dihydro-1-benzofuran-2-
yl]methyl}amine,
(±)-{[7-(2,5-difluorophenyl)-5-(trifluoromethyl)-2,3-dihydro-1-benzofuran-2-yl]methyl}amine,
(±)-{[7-(2,5-dichlorophenyl)-5-(trifluoromethyl)-2,3-dihydro-1-benzofuran-2-yl]methyl}amine,
(±)-{[7-(2,6-dimethylphenyl)-5-(trifluoromethyl)-2,3-dihydro-1-benzofuran-2-yl]methyl}amine,
(±)-{[7-(2-fluorophenyl)-5-methoxy-2,3-dihydro-1-benzofuran-2-yl]methyl}amine,
(±)-{[7-(2-chlorophenyl)-5-methoxy-2,3-dihydro-1-benzofuran-2-yl]methyl}amine,
(±)-{[7-(2-methylphenyl)-5-methoxy-2,3-dihydro-1-benzofuran-2-yl]methyl}amine,
(±)-{[7-(2-methoxyphenyl)-5-methoxy-2,3-dihydro-1-benzofuran-2-yl]methyl}amine,
(±)-{[7-(2,3-difluorophenyl)-5-methoxy-2,3-dihydro-1-benzofuran-2-yl]methyl}amine,
(±)-{[7-(2,3-dichlorophenyl)-5-methoxy-2,3-dihydro-1-benzofuran-2-yl]methyl}amine,
(±)-{[7-(2,3-dimethylphenyl)-5-methoxy-2,3-dihydro-1-benzofuran-2-yl]methyl}amine,
(±)-{[7-(2,4-difluorophenyl)-5-methoxy-2,3-dihydro-1-benzofuran-2-yl]methyl}amine,
(±)-{[7-(2,4-dichlorophenyl)-5-methoxy-2,3-dihydro-1-benzofuran-2-yl]methyl}amine,
(±)-{[7-(2,5-difluorophenyl)-5-methoxy-2,3-dihydro-1-benzofuran-2-yl]methyl}amine,
(±)-{[7-(2,5-dichlorophenyl)-5-methoxy-2,3-dihydro-1-benzofuran-2-yl]methyl}amine,
(+)-{[7-(2,5-dichlorophenyl)-5-methoxy-2,3-dihydro-1-benzofuran-2-yl]methyl}amine,
(−)-{[7-(2,5-dichlorophenyl)-5-methoxy-2,3-dihydro-1-benzofuran-2-yl]methyl}amine,
(±)-{[7-(2,5-dimethylphenyl)-5-methoxy-2,3-dihydro-1-benzofuran-2-yl]methyl}amine,
(±)-{[7-(2,5-dimethoxylphenyl)-5-methoxy-2,3-dihydro-1-benzofuran-2-yl]methyl}amine,
(±)-{[7-(5-chloro-2-methoxyphenyl)-5-methoxy-2,3-dihydro-1-benzofuran-2-yl]methyl}amine,
(±)-{[7-(2,6-dimethylphenyl)-5-methoxy-2,3-dihydro-1-benzofuran-2-yl]methyl}amine,
(±)-[(N-methyl-1-[7-(2-methoxyphenyl)-2,3-dihydro-1-benzofuran-2-yl]methanamine,
(±)-[(N-methyl-1-[7-(2,3-dimethylphenyl)-2,3-dihydro-1-benzofuran-2-yl]methanamine,
(±)-[(N-methyl-1-[7-(2,3-dimethoxyphenyl)-2,3-dihydro-1-benzofuran-2-yl]methanamine,
(±)-[(N-methyl-1-[7-(2,4-difluorophenyl)-2,3-dihydro-1-benzofuran-2-yl]methanamine,
(±)-[(N-methyl-1-[7-(2,4-dichlorophenyl)-2,3-dihydro-1-benzofuran-2-yl]methanamine,
(±)-[(N-methyl-1-[7-(2,4-dimethoxyphenyl)-2,3-dihydro-1-benzofuran-2-yl]methanamine,
(±)-[(N-methyl-1-[7-(2,5-dimethylphenyl)-2,3-dihydro-1-benzofuran-2-yl]methanamine,
(±)-[(N-methyl-1-[7-(2,5-difluorophenyl)-2,3-dihydro-1-benzofuran-2-yl]methanamine,
(±)-[(N-methyl-1-[7-(2,5-dichlorophenyl)-2,3-dihydro-1-benzofuran-2-yl]methanamine,
(±)-[(N-methyl-1-[7-(2,6-dimethylphenyl)-2,3-dihydro-1-benzofuran-2-yl]methanamine,
(±)-[(N-methyl-1-[7-(2,6-dichlorophenyl)-2,3-dihydro-1-benzofuran-2-yl]methanamine,
(−)-{[7-(2,6-dichlorophenyl)-2,3-dihydro-1-benzofuran-2-yl]methyl}methylamine,
(+)-{[7-(2,6-dichlorophenyl)-2,3-dihydro-1-benzofuran-2-yl]methyl}methylamine,
(±)-[(N-methyl-1-[7-(2,3-difluorophenyl)-2,3-dihydro-1-benzofuran-2-yl]methanamine,
(±)-{[5-fluoro-7-(2-fluorophenyl)-2,3-dihydro-1-benzofuran-2-yl]methyl}methylamine,
(±)-{[5-fluoro-7-(2-chlorophenyl)-2,3-dihydro-1-benzofuran-2-yl]methyl}methylamine,
(±)-{[5-fluoro-7-(2-methylphenyl)-2,3-dihydro-1-benzofuran-2-yl]methyl}methylamine,
(−)-{[5-fluoro-7-(2-methylphenyl)-2,3-dihydro-1-benzofuran-2-yl]methyl}methylamine,
(+)-{[5-fluoro-7-(2-methylphenyl)-2,3-dihydro-1-benzofuran-2-yl]methyl}methylamine,
(±)-{[5-fluoro-7-(2-methoxyphenyl)-2,3-dihydro-1-benzofuran-2-yl]methyl}methylamine,
(±)-{[7-(2,3-dimethoxyphenyl)-5-fluoro-2,3-dihydro-1-benzofuran-2-yl]methyl}methylamine,
(±)-[7-(2,4-dichlorophenyl)-5-fluoro-2,3-dihydro-1-benzofuran-2-yl]methyl}methylamine,
(−)-[7-(2,4-dichlorophenyl)-5-fluoro-2,3-dihydro-1-benzofuran-2-yl]methyl}methylamine,
(+)-[7-(2,4-dichlorophenyl)-5-fluoro-2,3-dihydro-1-benzofuran-2-yl]methyl}methylamine,
(±)-{[7-(2,5-difluorophenyl)-5-fluoro-2,3-dihydro-1-benzofuran-2-yl]methyl}methylamine,
(±)-{[7-(2,5-dichlorophenyl)-5-fluoro-2,3-dihydro-1-benzofuran-2-yl]methyl}methylamine,
(+)-{[5-fluoro-7-(2,5-dichlorophenyl)-2,3-dihydro-1-benzofuran-2-yl]methyl}methylamine,
(−)-{[5-fluoro-7-(2,5-dichlorophenyl)-2,3-dihydro-1-benzofuran-2-yl]methyl}methylamine,
(±)-{[7-(2,5-dimethylphenyl)-5-fluoro-2,3-dihydro-1-benzofuran-2-yl]methyl}methylamine,
(±)-{[7-(2,6-dimethylphenyl)-5-fluoro-2,3-dihydro-1-benzofuran-2-yl]methyl}methylamine,
(±)-{[7-(2,6-dichlorophenyl)-5-fluoro-2,3-dihydro-1-benzofuran-2-yl]methyl}methylamine,
(±)-{[5-fluoro-7-(5-methoxy-2-methylphenyl)-2,3-dihydro-1-benzofuran-2-
yl]methyl}methylamine,
(±)-{[5-fluoro-7-(2-methoxy-5-methylphenyl)-2,3-dihydro-1-benzofuran-2-
yl]methyl}methylamine,
(±)-{[7-(5-chloro-2-methoxyphenyl)-5-fluoro-2,3-dihydro-1-benzofuran-2-
yl]methyl}methylamine,
(±)-[(5-chloro-7-(2-methoxyphenyl)-2,3-dihydro-1-benzofuran-2-yl]methyl}methylamine,
(±)-{[5-chloro-7-(2,6-dimethylphenyl)-2,3-dihydro-1-benzofuran-2-yl]methyl}methylamine,
(±)-[(5-chloro-7-(2-fluorophenyl)-2,3-dihydro-1-benzofuran-2-yl]methyl}methylamine,
(±)-{[5-chloro-7-(2,3-difluorophenyl)-2,3-dihydro-1-benzofuran-2-yl]methyl}methylamine,
(±)-{[5-chloro-7-(2,3-dichlorophenyl)-2,3-dihydro-1-benzofuran-2-yl]methyl}methylamine,
(±)-{[5-chloro-7-(2,3-dimethylphenyl)-2,3-dihydro-1-benzofuran-2-yl]methyl}methylamine,
(±)-{[5-chloro-7-(2,3-dimethoxyphenyl)-2,3-dihydro-1-benzofuran-2-yl]methyl}methylamine,
(±)-{[5-chloro-7-(2,4-difluorophenyl)-2,3-dihydro-1-benzofuran-2-yl]methyl}methylamine,
(±)-{[5-chloro-7-(2,4-dichlorophenyl)-2,3-dihydro-1-benzofuran-2-yl]methyl}methylamine,
(±)-{[5-chloro-7-(2,4-dimethoxyphenyl)-2,3-dihydro-1-benzofuran-2-yl]methyl}methylamine,
(±)-{[5-chloro-7-(2,5-difluorophenyl)-2,3-dihydro-1-benzofuran-2-yl]methyl}methylamine,
(±)-{[5-chloro-7-(2,5-dichlorophenyl)-2,3-dihydro-1-benzofuran-2-yl]methyl}methylamine,
(±)-{[5-chloro-7-(5-chloro-2-methoxyphenyl)-2,3-dihydro-1-benzofuran-2-
yl]methyl}methylamine,
(±)-{[7-(2-fluorophenyl)-5-methyl-2,3-dihydro-1-benzofuran-2-yl]methyl}methylamine,
(±)-{[7-(2-chlorophenyl)-5-methyl-2,3-dihydro-1-benzofuran-2-yl]methyl}methylamine,
(±)-{[7-(2-methoxyphenyl)-5-methyl-2,3-dihydro-1-benzofuran-2-yl]methyl}methylamine,
(±)-{[7-(2,3-dimethoxyphenyl)-5-methyl-2,3-dihydro-1-benzofuran-2-yl]methyl}methylamine,
(±)-{[7-(2,4-dichlorophenyl)-5-methyl-2,3-dihydro-1-benzofuran-2-yl]methyl}methylamine,
(±)-{[7-(2,5-dichlorophenyl)-5-methyl-2,3-dihydro-1-benzofuran-2-yl]methyl}methylamine,
(+)-{[7-(2,5-dichlorophenyl)-5-methyl-2,3-dihydro-1-benzofuran-2-yl]methyl}methylamine,
(−)-{[7-(2,5-dichlorophenyl)-5-methyl-2,3-dihydro-1-benzofuran-2-yl]methyl}methylamine,
(±)-{[7-(2,6-dimethylphenyl)-5-methyl-2,3-dihydro-1-benzofuran-2-yl]methyl}methylamine,
(±)-{[7-(2,6-dichlorophenyl)-5-methyl-2,3-dihydro-1-benzofuran-2-yl]methyl}methylamine,
(±)-{[7-(2-fluorophenyl)-5-methoxy-2,3-dihydro-1-benzofuran-2-yl]methyl}methylamine,
(±)-{[7-(2-chlorophenyl)-5-methoxy-2,3-dihydro-1-benzofuran-2-yl]methyl}methylamine,
(±)-{[7-(2-methylphenyl)-5-methoxy-2,3-dihydro-1-benzofuran-2-yl]methyl}methylamine,
(±)-{[7-(2,3-difluorophenyl)-5-methoxy-2,3-dihydro-1-benzofuran-2-yl]methyl}methylamine,
(±)-{[7-(2,3-dichlorophenyl)-5-methoxy-2,3-dihydro-1-benzofuran-2-yl]methyl}methylamine,
(±)-{[7-(2,3-dimethylphenyl)-5-methoxy-2,3-dihydro-1-benzofuran-2-yl]methyl}methylamine,
(±)-{[7-(2,4-difluorophenyl)-5-methoxy-2,3-dihydro-1-benzofuran-2-yl]methyl}methylamine,
(±)-{[7-(2,5-difluorophenyl)-5-methoxy-2,3-dihydro-1-benzofuran-2-yl]methyl}methylamine,
(±)-{[7-(2,5-dichlorophenyl)-5-methoxy-2,3-dihydro-1-benzofuran-2-yl]methyl}methylamine,
(±)-{[7-(2,5-dimethylphenyl)-5-methoxy-2,3-dihydro-1-benzofuran-2-yl]methyl}methylamine,
(±)-{[7-(2,5-dimethoxyphenyl)-5-methoxy-2,3-dihydro-1-benzofuran-2-yl]methyl}methylamine,
(±)-{[7-(5-chloro-2-methoxyphenyl)-5-methoxy-2,3-dihydro-1-benzofuran-2-
yl]methyl}methylamine,
(±)-{[7-(2,6-dimethylphenyl)-5-methoxy-2,3-dihydro-1-benzofuran-2-yl]methyl}methylamine,
(±)-N-methyl-1-[7-(2,3-difluorophenyl)-5-(trifluoromethyl)-2,3-dihydro-1-benzofuran-2-
yl]methanamine,
(±)-N-methyl-1-[7-(2,3-dichlorophenyl)-5-(trifluoromethyl)-2,3-dihydro-1-benzofuran-2-
yl]methanamine,
(±)-N-methyl-1-[7-(2,4-dimethoxyphenyl)-5-(trifluoromethyl)-2,3-dihydro-1-benzofuran-2-
yl]methanamine,
(+){[7-(2,6-dichlorophenyl)-5-fluoro-2,3-dihydro-1-benzofuran-2-yl]methyl}methylamine,
(−){[7-(2,6-dichlorophenyl)-5-fluoro-2,3-dihydro-1-benzofuran-2-yl]methyl}methylamine,
(R)-[7-(2-chloro-phenyl)-(5-Fluoro-2,3-dihydro-benzofuran-2-ylmethyl)methyl-amine,
(R)-[7-(2,6-dichloro-phenyl)-5-Fluoro-2,3-dihydro-benzofuran-2-ylmethyl]ethylamine,
(R)-[7-(2,6-dichloro-phenyl)-5-Fluoro-2,3-dihydro-benzofuran-2-ylmethyl]dimethylamine,
{[(2R)-7-(5-chloro-2-methylphenyl)-5-fluoro-2,3-dihydro-1-benzofuran-2-yl]methyl}amine,
{[(2R)-7-(4-chloro-2-methylphenyl)-5-fluoro-2,3-dihydro-1-benzofuran-2-yl]methyl}amine,
(−)-{[7-(2,6-dichlorophenyl)-5-fluoro-2,3-dihydro-1-benzofuran-2-yl]methyl}amine,
(+)-{[7-(2,6 dichlorophenyl)-5-fluoro-2,3-dihydro-1-benzofuran-2-yl]methyl}amine,
(±)-{2-[6-chloro-7-(2-chlorophenyl)-2,3-dihydro-1-benzofuran-2-yl]ethyl}amine,
(±)-{2-[7-(2,6-dichlorophenyl)-5-fluoro-2,3-dihydro-1-benzofuran-2-yl]ethyl}amine,
(±)-{2-[7-(2-methoxyphenyl)-5-methoxy-2,3-dihydro-1-benzofuran-2-yl]ethyl}amine,
(±)-{N-methyl-1-[(7-(2,4,6-trichlorophenyl)-2,3-dihydro-1-benzofuran-2-yl]methanamine,
(+)-{N-methyl-1-[(7-(2,4,6-trichlorophenyl)-2,3-dihydro-1-benzofuran-2-yl]methanamine,
(−)-{N-methyl-1-[(7-(2,4,6-trichlorophenyl)-2,3-dihydro-1-benzofuran-2-yl]methanamine,
(+)-{[7-(2,6-dimethylphenyl)-5-fluoro-2,3-dihydro-1-benzofuran-2-yl]methyl}methylamine,
(−)-{[7-(2,6-dimethylphenyl)-5-fluoro-2,3-dihydro-1-benzofuran-2-yl]methyl}methylamine,
(−)-{[7-(2,6-dimethylphenyl)-5-methoxy-2,3-dihydro-1-benzofuran-2-yl]methyl}methylamine,
(+)-{[7-(2,6-dimethylphenyl)-5-methoxy-2,3-dihydro-1-benzofuran-2-yl]methyl}methylamine,
(+)-{[5-chloro-7-(2,5-dichlorophenyl)-2,3-dihydro-1-benzofuran-2-yl]methyl}methylamine,
(−)-{[5-chloro-7-(2,5-dichlorophenyl)-2,3-dihydro-1-benzofuran-2-yl]methyl}methylamine,
(−)-{[5-chloro-7-(2,6-dimethylphenyl)-2,3-dihydro-1-benzofuran-2-yl]methyl}methylamine,
(+)-{[5-chloro-7-(2,6-dimethylphenyl)-2,3-dihydro-1-benzofuran-2-yl]methyl}methylamine,
(+)-{[7-(2,3-dimethoxyphenyl)-5-methyl-2,3-dihydro-1-benzofuran-2-yl]methyl}methylamine,
(−)-{[7-(2,3-dimethoxyphenyl)-5-methyl-2,3-dihydro-1-benzofuran-2-yl]methyl}methylamine,
(−)-{[7-(2,3-dimethoxyphenyl)-5-fluoro-2,3-dihydro-1-benzofuran-2-yl]methyl}methylamine, or
(+)-{[7-(2,3-dimethoxyphenyl)-5-fluoro-2,3-dihydro-1-benzofuran-2-yl]methyl}methylamine;
or a pharmaceutically acceptable salt thereof.

Compounds of formula I for use in accordance with the present invention may be obtained or produced according to any available means including methods described in detail in U.S. patent application Ser. No. 10/970,714, filed Oct. 21, 2004, and U.S. provisional patent application Ser. Nos. 60/621,023, filed Oct. 21, 2004, and 60/621,024, filed Oct. 21, 2004, the entirety of each of which is hereby incorporated herein by reference.

Without wishing to be bound by any particular theory, the present inventors note that compounds of formula I are highly specific agonists, or partial agonists, of the 5HT_{2C} receptor. The present invention encompasses the recognition that this unique affinity and selectivity displayed by compounds of formula I renders them particularly useful for treating cognitive, and other disorders. The present invention also contemplates that compounds of formula I are associated with a rapid onset of action. In addition, compounds of formula I lack the side-effect of sexual dysfunction.

2. Pharmaceutical Compositions

Compounds of formula I may be administered neat in order to treat a cognitive, or other disorder, in accordance with the present invention. More commonly, however, they are administered in the context of a pharmaceutical composition, that contains a therapeutically effective amount of one or more compound of formula I together with one or more other ingredients known to those skilled in the art for formulating pharmaceutical compositions.

As used herein, the terms “pharmaceutically effective amount” or “therapeutically effective amount” mean the total amount of each active component of the pharmaceutical composition or method that is sufficient to show a meaningful patient benefit, i.e., treatment, prevention or amelioration of a cognitive, or other disorder. When applied to an individual active ingredient, administered alone, the term refers to that ingredient alone. When applied to a combination, the term refers to combined amounts of the active ingredients that result in the therapeutic effect, whether administered in combination, serially or simultaneously.

In certain embodiments of the invention, compounds of formula I are administered with a daily dose in the range of about 0.5 to about 500 mg, or about 1 mg to about 500 mg. Doses may be administered as a single regimen, such as only prior to bedtime or before travel, or as a continuous regimen divided by two or more doses over the course of a day. The dosage levels and other dosage levels herein are for the average human subject having a weight range of about 65 to 70 kg. The skilled person will readily be able to determine the dosage levels required for a subject whose weight falls outside this range, such as children and the elderly.

The dosage of the combination of the invention in such formulations will depend on its potency, but can be expected to be in the range of from 1 to 500 mg of 5-HT_{2C} receptor agonist for administration up to three times a day. In some embodiments, the dose may be in the range of about 10 to 100 mg (e.g. 10, 25, 50 and 100 mg) of 5-HT_{2C} receptor agonist which can be administered once, twice or three times a day (preferably once). However the precise dose will be as determined by the prescribing physician and will depend on the age and weight of the subject and severity of the symptoms.

Additional ingredients useful in preparing pharmaceutical compositions in accordance with the present invention include, for example, carriers (e.g., in solid or liquid form), flavoring agents, lubricants, solubilizers, suspending agents, fillers, glidants, compression aids, binders, tablet-disintegrating agents, encapsulating materials, emulsifiers, buffers, preservatives, sweeteners, thickening agents, coloring agents, viscosity regulators, stabilizers or osmo-regulators, or combinations thereof.

Solid pharmaceutical compositions preferably contain one or more solid carriers, and optionally one or more other additives such as flavoring agents, lubricants, solubilizers, suspending agents, fillers, glidants, compression aids, binders or tablet-disintegrating agents or an encapsulating material. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes or ion exchange resins, or combinations thereof. In powder pharmaceutical compositions, the carrier is preferably a finely divided solid which is in admixture with the finely divided active ingredient. In tablets, the active ingredient is generally mixed with a carrier having the necessary compression properties in suitable proportions, and optionally, other additives, and compacted into the desired shape and size. Solid pharmaceutical compositions, such as powders and tablets, preferably contain up to 99% of the active ingredient.

In certain embodiments, a compound of formula I is provided in a disintegrating tablet formulation suitable for pediatric administration.

Liquid pharmaceutical compositions preferably contain one or more compounds of formula I and one or more liquid carriers to form solutions, suspensions, emulsions, syrups, elixirs, or pressurized compositions. Pharmaceutically acceptable liquid carriers include, for example water, organic solvents, pharmaceutically acceptable oils or fat, or combinations thereof. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators, or combinations thereof. If the liquid formulation is intended for pediatric use, it is generally desirable to avoid inclusion of alcohol.

Examples of liquid carriers suitable for oral or parenteral administration include water (preferably containing additives such as cellulose derivatives such as sodium carboxymethyl cellulose), alcohols or their derivatives (including monohydric alcohols or polyhydric alcohols such as glycols) or oils (e.g., fractionated coconut oil and arachis oil). For parenteral administration the carrier can also be an oily ester such as ethyl oleate and isopropyl myristate. The liquid carrier for pressurized compositions can be halogenated hydrocarbons or other pharmaceutically acceptable propellant.

In certain embodiments, a liquid pharmaceutical composition is provided wherein said composition is suitable for pediatric administration. In other embodiments, the liquid composition is a syrup or suspension.

Liquid pharmaceutical compositions which are sterile solutions or suspensions can be administered parenterally, for example by, intramuscular, intraperitoneal, epidural, intrathecal, intravenous or subcutaneous injection. Pharmaceutical compositions for oral or transmucosal administration may be either in liquid or solid composition form.

In some embodiments of the invention, pharmaceutical compositions are provided in unit dosage form, such as tablets or capsules. In such form, the composition is sub-divided in unit dose containing appropriate quantities of the active ingredient(s). The unit dosage forms can be packaged compositions, for example packeted powders, vials, ampoules, pre-filled syringes or sachets containing liquids. The unit dosage form can be, for example, a capsule or tablet itself, or it can be an appropriate number of any such compositions in package form.

Thus, the present invention also provides a pharmaceutical composition in unit dosage form for a cognitive, or other disorder, in a patient, where the composition contains a therapeutically effective unit dosage of at least one compound of formula I. As one skilled in the art will recognize, the preferred therapeutically effective unit dosage will depend on for example the method of administration. For example, a unit dosage for oral administration often ranges from about 0.5 mg to about 500 mg and more typically from about 1 mg to about 500 mg of the compound of formula I.

The present invention also provides a therapeutic package for dispensing the compounds of formula 1 to a patient being treated for a cognitive, or other disorder as described herein. In some embodiments, the therapeutic package contains one or more unit dosages of the compound of formula I, a container containing the one or more unit dosages, and labeling directing the use of the package for treating a cognitive, or other disorder, in a patient. In certain embodiments, the unit dose is in tablet or capsule form. In some cases, each unit dosage is a therapeutically effective amount.

3. Other Pharmaceutical Agents

According to the present invention, compounds of formula I may be administered alone to treat one or more cognitive, or other disorders, or alternatively may be administered in combination with (whether simultaneously or sequentially) one or more other pharmaceutical agents useful to treat one or more cognitive, or other disorders, as described herein. Alternatively or additionally, the compounds of formula I may be administered in combination with one or more other pharmaceutical agents useful in the treatment or prevention of one or more other symptoms, disorders, or diseases suffered by the individual in need of treatment of one or more cognitive, or other disorders, as described herein.

An exemplary listing of pharmaceutically active agents that may be administered in conjunction with one or more compounds of formula I in accordance with the present invention can be found in the Physicians' Desk Reference, 55 Edition, 2001, published by Medical Economics Co., Inc., Montvale, N.J. For many of these listed agents, pharmaceutically effective dosages and regimens are known in the art; many are presented in the Physicians' Desk Reference itself.

4. Uses

Methods of this invention are useful for treating one or more cognitive, or other disorders, as described herein, in a patient.

In certain embodiments, the present invention provides a method of treating one or more intellectual deficit disorders comprising administering a compound of the present invention. In other embodiments, such intellectual deficit disorders include dementia, such as dementia of aging, vascular dementia, mild cognitive impairment, age-related cognitive decline, and mild neurocognitive disorder; Alzheimer's disease, and memory deficit, attention deficit disorders (ADD, also known as Attention Deficit Hyperactivity Disorder or ADHD) in both children and adults. In certain embodiments, the present invention provides a method of treating ADD and/or ADHD in a pediatric patient comprising administering to said patient a compound of formula I or pharmaceutical composition thereof.

In other embodiments, the present invention provides a method of treating one or more cognition disorders. According to another aspect, the cognition disorder is a learning disorder. Such learning disorders are known in the art and include autism, dyslexia, Asperger's syndrome, a neurobiological disorder similar to autism and characterized by serious deficits in social and communication skills; specific learning disability, a disorder in one or more of the basic psychological processes involved in understanding or in using spoken or written language, which may manifest itself in an imperfect ability to listen, think, speak, read, write, spell or to do mathematical calculations; dysgraphia, a disorder that causes difficulty with forming letters or writing within a defined space; dyscalculia, a disorder that causes people to have problems doing arithmetic and grasping mathematical concepts; dyspraxia, a problem with the body's system of motion that interferes with a person's ability to make a controlled or coordinated physical response in a given situation; visual perceptual deficit, difficulty receiving and/or processing accurate information from the sense of sight, although there is nothing wrong with vision; and auditory perceptual deficit, difficulty receiving accurate information through auditory means, even though there is no problem with hearing.

In certain embodiments, the present invention provides a method for treating one or more impulsivity disorders (e.g. borderline personality disorder), disruptive behavior disorders, or impulse control disorders. In certain embodiments, the present invention provides a method for treating Tourette's Syndrome (TS), an inherited, neurological disorder characterized by repeated and involuntary body movements (tics) and/or uncontrollable vocal sounds. In other embodiments, the present invention provides a method for treating trichotillomania.

According to another aspect, the present invention provides a method for treating one or more behavioral addictions and addictive disorders. Behavioral addictions and addictive disorders result from the intoxication one senses from the release of brain chemicals (e.g., serotonin, adrenaline, epinepherine, etc.) during certain activities. Such disorders are known in the art and include gambling, sex addiction, eating disorders, spending addiction, rage/anger, workaholism, exercise addiction, risk taking addictions (e.g. kleptomania and pyromania), and perfectionism, to name a few.

In certain embodiments, a compound of the present invention is administered in combination with one or more cognitive improvement agents. Such agents are well known in the art and include donepezil hydrochloride (Aricept™) and other acetylcholinesterase inhibitors; galantamine, neuroprotective agents (e.g., memantine); ADD/ADHD agents (e.g., Ritalin™, Strattera™, Concerta™ and Adderall™), and methylphenidate.

As 5-HT_{2C} modulators, compounds of the present invention are useful for treating a variety of disorders. Such disorders include premenstrual syndrome (PMS), premenstrual dysphoric disorder (PMDD), or late luteal phase syndrome, motion or motor disorders such as Parkinson's disease; chronic fatigue syndrome, anorexia nervosa, disorders of sleep (e.g., sleep apnea), and mutism.

Premenstrual dysphoric disorder, or PMDD, is a severe form of PMS. Like PMS, PMDD typically occurs the week before the onset of menstruation and disappears a few days after. PMDD is characterized by severe monthly mood swings and physical symptoms that interfere with everyday life, especially a woman's relationships with her family and friends. PMDD symptoms go far beyond what are considered manageable or normal premenstrual symptoms.

PMDD is a combination of symptoms that may include irritability, depressed mood, anxiety, sleep disturbance, difficulty concentrating, angry outbursts, breast tenderness and bloating. The diagnostic criteria emphasize symptoms of depressed mood, anxiety, mood swings or irritability. The condition affects up to one in 20 American women who have regular menstrual periods. According to another embodiment, the present invention provides a method for treating one or more symptoms associated with PMDD.

Selective serotonin reuptake inhibitors (SSRIs) are the current preferred method for treating symptoms associated with PMDD. According to another aspect, the present invention provides a method for treating PMDD, or one or more symptoms associated with PMDD, by administering a compound of formula I in combination with an SSRI. In certain embodiments, the SSR¹ is fluoxetine, venlafaxine, paroxetine, duloxetine, or sertraline.

Inventive methods involve delivery of compounds of formula I via any appropriate route of administration including, for example, oral, buccal, sublingual, rectal, nasal, parenteral, intravenous, or other modes. In general, the compounds may be formulated for immediate, delayed, modified, sustained, pulsed, or controlled-release delivery.

For inventive methods utilizing oral delivery, such delivery may be accomplished using solid or liquid formulations, for example in the form of tablets, capsules, multi-particulates, gels, films, ovules, elixirs, solutions or suspensions. In certain embodiments, the compounds are administered as oral tablets or capsules or neat compound or powdered or granular pharmaceutical formulations. Such preparations may be mixed chewable or liquid formulations or food materials or liquids if desirable, for example to facilitate administration to children, to individuals whose ability to swallow tablets is compromised, or to animals. Examples of oral formulations contained in hard gelatin capsules can include those in which the active compound comprises from about 45% to 50%, by weight, of the formulation. Microcrystalline cellulose comprises from about 43% to about 47%, povidone comprises from about 3% to about 4%, and silicon dioxide and magnesium stearate each comprise from about 0.3% to about 0.7%, each by weight.

Modified release and pulsatile release oral dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release oral dosage forms may contain one or a combination of release rate modifying excipients. Release rate modifying excipients may be present both within the dosage form i.e., within the matrix, and/or on the dosage form, i.e., upon the surface or coating.

Fast dispersing or dissolving dosage oral formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

For inventive methods utilizing intravenous delivery, such administration may be, for example, intracavernous, intravenous, intra-arterial, intraperitoneal, intrathecal, intraventricular, intraurethral, intrastemal, intracranial, intramuscular or subcutaneous, or via by infusion or needleless injection techniques. For such parenteral administration, the compounds of formula I may be prepared and maintained in conventional lyophylized formulations and reconstituted prior to administration with an intravenously acceptable saline solution, such as a 0.9% saline solution. The pH of the intravenous formulation can be adjusted, as is known in the art, with an intravenous and pharmaceutically acceptable acid, such as methanesulfonic acid.

The compounds of formula I can also be administered intranasally or by inhalation and are conveniently delivered in the form of a dry powder inhaler or an aerosol spray presentation from a pressurized container, pump, spray, atomizer or nebuliser, with or without the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurized aerosol, the dosage unit may be determined by providing a valve to deliver a metered amount. The pressurized container, pump, spray, atomizer or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the compounds of the invention and a suitable powder base such as lactose or starch.

Aerosol or dry powder formulations are preferably arranged so that each metered dose or “puff” contains from 1 .mu.g to 50 mg of a compound of the invention for delivery to the patient. The overall daily dose with an aerosol will be in the range of from 1 .mu.g to 50 mg which may be administered in a single dose or, more usually, in divided doses throughout the day.

Alternatively, the compounds of formula I can be administered in the form of a suppository or pessary, or they may be applied topically in the form of a gel, hydrogel, lotion, solution, cream, ointment or dusting powder. The compounds of the invention may also be dermally or transdermally administered, for example, by the use of a skin patch, depot or subcutaneous injection. They may also be administered by the pulmonary or rectal routes.

For application topically to the skin, the compounds of formula I can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compounds of formula I may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in published international patent applications WO91/11172, WO94/02518 and WO98/55148.

### EXEMPLIFICATION

### Example 1

Compound 1, (7-(2,6-dichlorophenyl)-5-fluoro-2,3-dihydrobenzofuran-2-yl)methanamine, was used to exemplify the effect of compounds of the present invention on on compulsive behavior.

Introduction

Compound 1 is a potent full agonist at the 5-HT_{2C} receptor (Ki=0.37 nM; EC₅₀=0.4 nM). Several lines of evidence suggest that 5-HT_{2C} agonists may be effective treatments for obsessive-compulsive disorder (OCD). For example, 5-HT_{2C} agonists have been shown to be effective in animal models of compulsive behavior such as schedule-induced polydipsia (SIP), 8-OH-DPAT-induced scratching in squirrel monkeys, marble burying, and excessive eating of palatable foods. Furthermore, 5-HT_{2C} receptor knockout mice exhibit compulsive-like behaviors.

The present studies were conducted to evaluate the effects of Compound 1 in a schedule-induced polydipsia model of obsessive-compulsive disorder in rats. In this model, a food pellet is delivered once per minute for a two-hour period. A water bottle is available in the test chamber. Under this schedule, water intake is tremendously increased relative to animals that receive 120 food pellets at the start of a session and are given two hours to eat and drink. This excessive manifestation of a normal behavior (drinking) provides face validity to the model. Moreover, since the rats are not water deprived, the adjunctive drinking does not serve a physiological function. Most importantly, clinically effective drugs for the treatment of obsessive-compulsive disorder decrease adjunctive drinking.

Materials and Methods

Animals The animals used in these studies are as set forth in the table below.

Schedule-Induced Polydipsia Test Animals

| Descriptor | Description |
|---|---|
| Species: | Rat |
| Strain: | Sprague-Dawley |
| Source: | Charles River Laboratories |
| No. of Animals Per Group: | 6 |
| Total No. of Animals: | 6 |
| Age and Sex: | Age = unknown, Sex = Male |
| Weight: | 300-400 g |
| Acclimation Period: | Minimum of 2 weeks prior to training; |
| | >8 weeks of training prior to testing |

Male Sprague-Dawley rats weighing 300-400 grams were individually housed in an AALAC-accredited facility that was maintained on a 12-h light dark cycle (lights on at 0600 h) with freely available water. All in vivo studies were performed in accordance to the Guide for the Care and Use of Laboratory Animals as adopted and promulgated by the National Institutes of Health (Pub. 85-23, 1985). Rats were maintained at approximately 85% of their free-feeding body weights by food presented during the session and by post-session feeding.

Procedure Experimental sessions were conducted in a standard operant conditioning chamber placed inside a ventilated sound-attenuating shell that was equipped with white noise to mask extraneous sounds (Med Associates, Georgia, Vt.). A food trough was on the front panel of the chamber and a water bottle was mounted to the side panel. Experimental sessions were 120 minutes in duration and were conducted five days per week (Monday through Friday). Test sessions occurred generally on Tuesdays and Fridays, with the remaining days serving as control days. For each session, one food pellet (Bioserv, 45 mg precision dustless pellet) was dispensed into the food trough each minute for the duration of the session, for a total of 120 pellets being delivered. This schedule of food presentation elicits an abnormal adjunctive drinking behavior in rats such that they consume and exaggerated volume of water, approximately 5-10 fold larger than under normal conditions. Compound 1 or vehicle was administered immediately prior to the start of the experimental session. Each dose of Compound 1 was tested on a separate day, with the appropriate control sessions being conducted prior to each test. At the end of the test session, the volume of water and the number of food pellets that each rat consumed were measured.

In order to determine if Compound 1 would affect normal eating and drinking behavior, Compound 1 or vehicle was tested under modified conditions. Under these modified conditions, 120 food pellets were placed into the food trough at the beginning of the session and no further food pellets were dispensed. At the end of the test session, the volume of water and the number of food pellets consumed by each rat were measured as described earlier.

Water consumption was calculated and expressed as percent (%) of vehicle control intake. Results were analyzed using a one-way ANOVA with least significant difference (LSD) post-hoc test. Statistically significant decreases in adjunctive drinking were defined as p<0.05 relative to mean vehicle control values. Where appropriate, ED₅₀ values (dose that decreases adjunctive water intake to 50% of vehicle values) were calculated using a logistic dose response analysis.

Results and Discussion

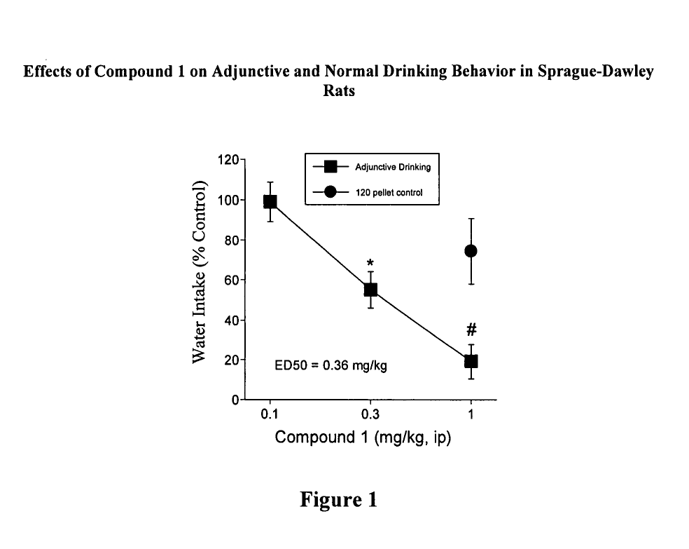

The effect of Compound 1 (0.1-1 mg/kg, i.p.) on schedule-induced polydipsia was evaluated. Compound 1 produced a dose-dependent decrease in adjunctive drinking behavior (F_{(3,20)}=16.91, p<0.0001). Post hoc tests revealed that doses of 0.3 and 1 mg/kg of Compound 1 produced statistically significant reductions in adjunctive drinking behavior of 45% (p=0.003) and 81% (p<0.0001) respectively. The ED₅₀ value was 0.36 mg/kg (95% CI: 0.22-0.57 mg/kg). At the highest dose of 1 mg/kg, one of the six rats did not eat all of the food pellets presented. When tested under the modified procedure to assess effects on normal feeding and drinking behavior, 1 mg/kg of Compound 1 decreased water consumption by 26% relative to vehicle controls and two of the six rats tested failed to eat all of the food pellets presented (FIG. 1).

The present studies demonstrate that Compound 1 produced dose-dependent decreases in adjunctive drinking following i.p. administration. The anti-OCD-like effects of Compound 1 on SIP occurred following an acute administration. In contrast, the effects of serotonin reuptake inhibitors generally require chronic administration in this model. Taken together with previous studies demonstrating that 5-HT_{2C} agonists are effective in animal models of compulsive behaviors, these results suggest that Compound 1 may be an effective treatment for obsessive-compulsive disorder and may have more rapid onset than current pharmacotherapies.

### Example 2

### Assessment of Effect on Acetylcholine and Glutamate Levels

Compound 1 was used to exemplify the effect of compounds of the present invention on rat brain acetylcholine and glutamate using in vivo microdialysis.

Materials and Methods

Animals: Adult male Sprague-Dawley rats (Charles River, Wilmington, Mass.) weighing 280-350 g at the time of surgery were used for all microdialysis experiments. Adult male Long-Evans rats (Charles River, Wilmington, Mass.) weighing 180-220 g were used for NOR studies. Rats were housed individually for recognition memory studies. All animals had free access to food and water and were group housed in the AAALAC-accredited facility that was maintained on a 12 h light dark cycle (lights on at 0600 h) for at least 1 week prior to surgery or behavioral testing. All in vivo studies were performed in accordance to the ‘Guide for the Care and Use of Laboratory Animals’ as adopted and promulgated by the National Institutes of Health (Pub. 85-23, 1985). The animals used in microdialysis are listed in Table 2.1.

TABLE 2-1
Test Systems for Microdialysis

| | |
|---|---|
| Species: | Rat |
| Strain: | CD |
| Source: | Charles River |
| No. of Animals Per Group: | 6-8 |
| Total No. of Animals: | 80 |
| Age and Sex: | 60-75 days old; Male |
| Weight: | 250-400 g |
| Acclimation Period: | ≧6 days |

Stereotaxic Surgery Following induction of anesthesia with 3% halothane (Fluothane; Zeneca, Cheshire, UK), animals were secured in a stereotaxic frame with ear and incisor bars (David Kopf, Tujunga, Calif.). Anesthesia was maintained by continuous administration of halothane (1-2%) while a microdialysis guide cannula (CMA/12, CMA Microdialysis, Stockholm, Sweden) was implanted above the dorsal hippocampus (AP: −4.3 mm ML: −2.6 mm DV: −2.1 mm) or the medial prefrontal cortex (AP: 3.2 mm ML: −0.6 mm DV: −3.8 mm). The guide cannula was secured to the skull using dental acrylic (Plastics one, Roanoke, Va., USA) and two small stainless-steel screws (Plastics one, Roanoke, Va., USA). Following surgery, animals were individually housed in Plexiglas cages (45 cm sq.), with free access to food and water. The following day rats were used in microdialysis experiments.

Microdialysis Microdialysis probes (CMA 12/02; CMA Microdialysis, Stockholm, Sweden) were equilibrated according to manufacturer's specifications. Microdialysis probes were perfused with artificial cerebrospinal fluid (aCSF: 125 mM NaCl, 3 mM KCl, 0.8 mM MgCl₂, 1.85 mM CaCl₂, 1.54 mM Na₂HPO₄ and 0.225 mM NaH₂PO₄; pH 7.4) prior to insertion in the guide cannula. The microdialysis probe was then inserted via the guide cannula into the dorsal hippocampus or medial prefrontal cortex and perfused with aCSF at a flow rate of 0.5 μl/min. A 3 h stabilization period was allowed following probe insertion before dialysate sampling was initiated. Samples were collected every 40 min for acetylcholine analysis and immediately frozen on dry ice after collection. After baseline samples were collected (2 h), rats were dosed with Compound 1 or vehicle (t=0). Following dosing, dialysis samples were collected for 200 min. At the end of the experiment, animals were euthanized and probe placement was verified histologically. Data from animals with incorrect probe placement were discarded.

Analysis of Acetylcholine Dialysate levels of acetylcholine were determined using LC/MS/MS. The following conditions and equipment were used:
Instrument: Waters Micro mass spectrometer coupled with Agilent HP 1100
Column: Supelco LC-SCX, 2.1×150 mm, 5 mM
Mobile phase: A: 60/20/20 of H2O/buffer/ACN; B: 20/80 buffer/ACN
Buffer: 79.5 mM ammonium acetate, 63.5 mM ammonium formate, pH 4.0
Ionization mode: (+) ESI
MRM: Acetylcholine (ACh): 146 [symbol] 87 Cone=20 CID=15 eV
   Iso-ACh: 146 [symbol] 87 Cone=20 CID=15 eV
   (3-carboxypropyltrimethylammonium)
   IS: b-methyl-ACh: 160 [symbol] 101 Cone=20, CID=15 eV

Acetylcholine data were quantified using peak area against an internal standard and acquired using Masslynx software (Micromass, Beverly, Mass., USA). The mean of the concentration of baseline samples was calculated and denoted as 0%. All sample values were expressed as a percent change from this pre-injection mean baseline value (% change from baseline). Neurochemical data, excluding pre-injection values, were analyzed by a two-way analysis of variance (ANOVA) with repeated measures (time). All statistical analyses were performed using SAS (v 1.03) within Excel® (Microsoft).

High Performance Liquid Chromatography (HPLC) Analysis

Dialysate (10 μL) was collected and analyzed for extracellular glutamate concentrations. HPLC methods were conducted using the following methods: 10 μL dialysate containing glutamate was separated by HPLC. These units consisted of two Jasco PU-980 pumps (Jasco Ltd, Essex, U.K) as the gradient, a BAS sentinel autosampler (BAS) and a Jasco PF-920 fluorometer with excitation wavelength of 448 nm. The emission wavelength was 485 nm. Mobile phase A was 0.05 M acetate buffer (pH 6.5) with 20% methanol (V:V) and mobile phase B was a 0.05 M acetate buffer (pH 6.5) with 80% methanol (V:V). The gradient consisted of a linear transition from 80% mobile phase A to 0% in 18 minutes. The column was allowed to re-equilibrate for 10 min prior to each injection. Each sample was diluted 1:1 with normal Krebs solution containing 2.5 μM alpha-aminoadipic acid (>AA; final concentration 1.25 μM; internal standard). Samples containing >AA were derivatized with naphthalene 2,3-dicarboxaldehyde (NDA). Samples or standards were mixed with 30 mM boric acid buffer (pH 9.5) containing 20 μM cyanide, and 30 μM NDA in methanol (1:1:0.25; sample: borate: NDA) and were allowed to react for 10 minutes at 10° C. prior to fluorometric detection. Data were acquired using the Atlas software package (Labsystems, Gulph Mills, Pa.) for the PC.

Statistical Analysis of Results

The fmol concentrations of glutamate during the baseline samples were averaged and this value was denoted as 100%. Subsequent sample values were expressed as a percentage of this preinjection baseline value (% of control). Neurochemical data, excluding preinjection values, were analyzed by a two-way analysis of variance (ANOVA) with repeated measures (time). Post-hoc analyses were made using the Bonferroni/Dunns adjustment for multiple comparisons. All statistical calculations were performed using the Statview software application Abacus Concepts Inc., Berkeley, Calif.) for the PC.

Results and Discussion

Effect of Compound 1 on Acetylcholine in Medial Prefrontal Cortex

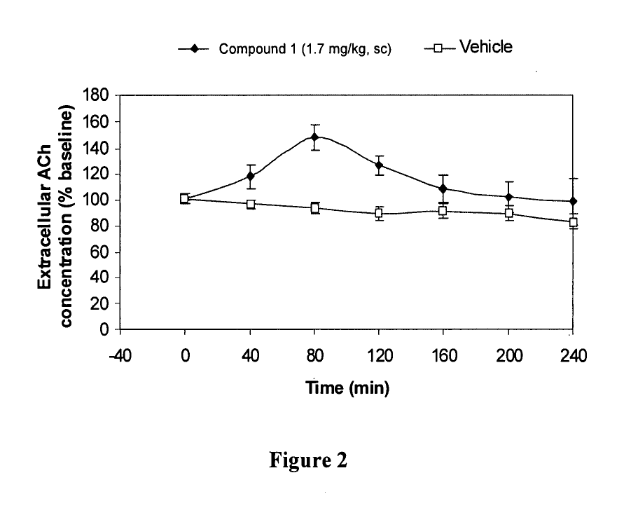

Administration of Compound 1 (1.7 mg/kg s.c.) produced a significant increase in acetylcholine in medial prefrontal cortex. The maximal increase was 48% with respect to baseline (FIG. 2).

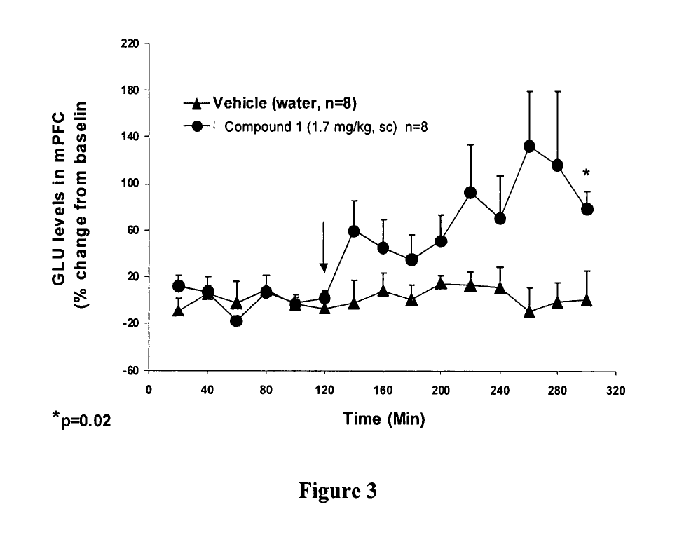

FIG. 3 shows the effect of Compound 1 (1.7 mg/kg, s.c.) on extracellular glutamate levels in the rat mPFC. We found that acute administration of the selective 5-HT_{2C} agonist significantly (F (1,15)=4.76, P=0.0454) elevated glutamate levels (132%) above vehicle-treated animals (FIG. 3).

Conclusion

The 5-HT_{2C} agonist, Compound 1 produces increases in acetylcholine and glutamate in the medial prefrontal cortex a brain area implicated in cognitive function. These data indicates a pro-cognitive neurochemical profile for Compound 1.

We claim:

1. A method of treating a cognitive disorder in a patient, comprising administering to said patient a therapeutically effective amount of a compound of formula I: 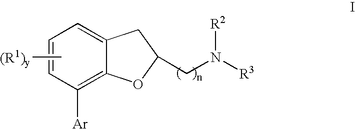
or a pharmaceutically acceptable salt thereof, wherein:
n is one or two;
each of R² and R³ is independently hydrogen, methyl, ethyl, 2-fluoroethyl, 2,2-difluoroethyl or cyclopropyl;
each R¹ is independently hydrogen, halogen, OH, lower alkyl, lower alkoxy, lower haloalkyl, lower haloalkoxy, or CN;
Ar is thienyl, furyl, pyridyl, or phenyl, wherein Ar is optionally substituted with one or more R^{x} subsituents;
each R^{x} is independently selected from halogen, OH, lower alkyl, lower alkoxy, lower haloalkyl, lower haloalkoxy, or CN; and
n is one or two.

2. The method according to claim 1, wherein one of R² and R³ is hydrogen and the other R² and R³ group is hydrogen, methyl, ethyl, 2-fluoroethyl, 2,2-difluoroethyl or cyclopropyl.

3. The method according to claim 2, wherein both of R² and R³ are hydrogen.

4. The method according to claim 1, wherein neither R² and R³ is hydrogen.

5. The method according to claim 1, wherein y is zero.

6. The method according to claim 1, wherein y is other than zero and at least one R¹ group is halogen.

7. The method according to claim 1, wherein y is one and R¹ is halogen, OH, lower alkyl, lower alkoxy, trifluoromethyl, trifluoromethoxy, or CN.

8. The method according to claim 7, wherein y is one and R¹ is fluoro or chloro.

9. The method according to claim 7, wherein said compound is of formula Ia or Ia′: 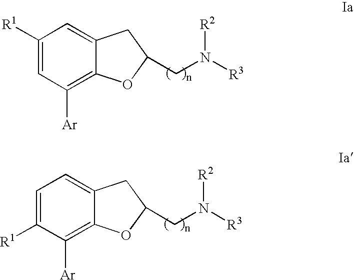
or a pharmaceutically acceptable salt thereof.

10. The method according to claim 1, wherein Ar is unsubstituted phenyl.

11. The method according to claim 1, wherein Ar is phenyl with at least one substituent in the ortho position.

12. The method according to claim 11, wherein Ar is phenyl with at least one substituent in the ortho position selected from halogen, lower alkyl, lower alkoxy, or trifluoromethyl.

13. The method according to claim 11, wherein said compound is of formula Ib or Ic: 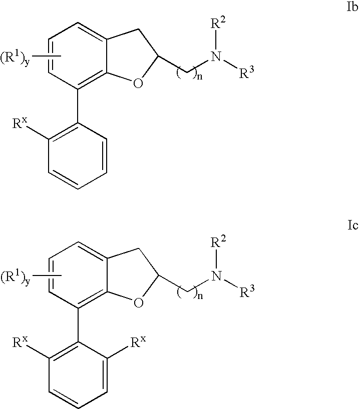
or a pharmaceutically acceptable salt thereof.

14. The method according to claim 13, wherein said compound is of formula Id or Ie: 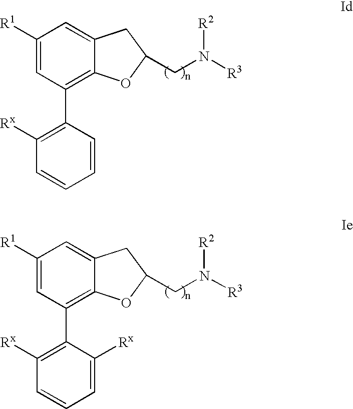
or a pharmaceutically acceptable salt thereof.

15. The method according to claim 1, wherein Ar is selected from:

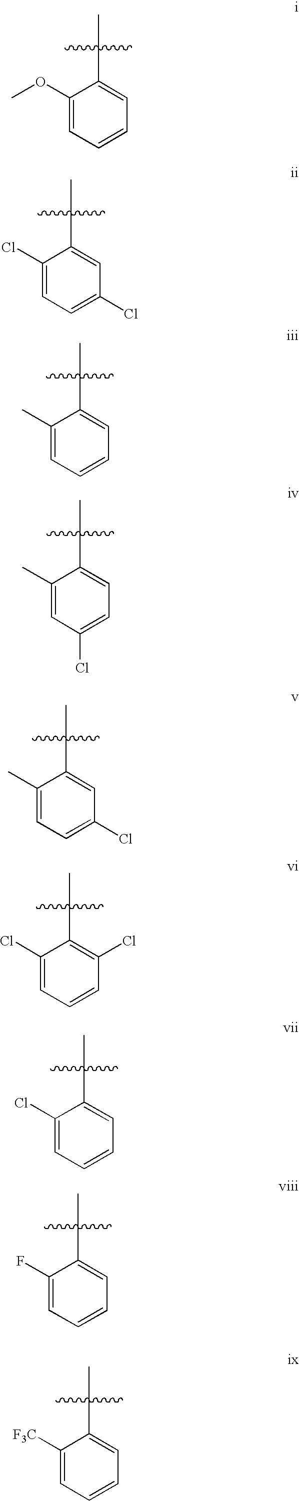

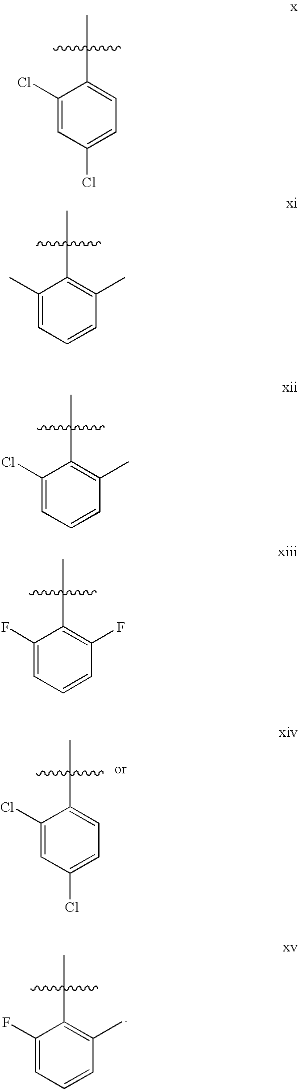

16. The method according to claim 1, wherein said compound is selected from:
(±)-1-{7-[3,5-bis(trifluoromethyl)phenyl]-2,3-dihydro-1-benzofuran-2-yl}methanamine,
(±)-1-[7-(3-chloro-4-fluorophenyl)-2,3-dihydro-1-benzofuran-2-yl]methanamine,
(±)-1-[7-(3,5-dichlorophenyl)-2,3-dihydro-1-benzofuran-2-yl]methanamine,
(±)-1-(7-phenyl-2,3-dihydro-1-benzofuran-2-yl)methanamine,
(+)-(1-(7-phenyl-2,3-dihydro-1-benzofuran-2-yl)methanamine,
(−)-1-(7-phenyl-2,3-dihydro-1-benzofuran-2-yl)methanamine,
(±)-1-[7-(3-methylphenyl)-2,3-dihydro-1-benzofuran-2-yl]methanamine,
(+)-1-[7-(3-methylphenyl)-2,3-dihydro-1-benzofuran-2-yl]methanamine,
(−)-1- [7-(3-methylphenyl)-2,3-dihydro-1-benzofuran-2-yl]methanamine,
(±)-1-(7-thien-3-yl-2,3-dihydro-1-benzofuran-2-yl)methanamine,
(+)-1-(7-thien-3-yl-2,3-dihydro-1-benzofuran-2-yl)methanamine,
(−)-1-(7-thien-3-yl-2,3-dihydro-1-benzofuran-2-yl)methanamine,
(±)-1-[7-(2-methylphenyl)-2,3-dihydro-1-benzofuran-2-yl]methanamine,
(+)-1-[7-(2-methylphenyl)-2,3-dihydro-1-benzofuran-2-yl]methanamine,
(−)-1-[7-(2-methylphenyl)-2,3-dihydro-1-benzofuran-2-yl]methanamine,
(±)-1-[7-(2-fluorophenyl)-2,3-dihydro-1-benzofuran-2-yl]methanamine,
(+)-1-[7-(2-fluorophenyl)-2,3-dihydro-1-benzofuran-2-yl]methanamine,
(−)-1-[7-(2-fluorophenyl)-2,3-dihydro-1-benzofuran-2-yl]methanamine,
(±)-1-{7-[2-(trifluoromethyl)phenyl]-2,3-dihydro-1-benzofuran-2-yl}methanamine,
(−)-1-{7-[2-(trifluoromethyl)phenyl]-2,3-dihydro-1-benzofuran-2-yl}methanamine,
(+)-1-{7-[2-(trifluoromethyl)phenyl]-2,3-dihydro-1-benzofuran-2-yl}methanamine,
(±)-1-[7-(2,6-dimethylphenyl)-2,3-dihydro-1-benzofuran-2-yl]methanamine,
(−)-1-[7-(2,6-dimethylphenyl)-2,3-dihydro-1-benzofuran-2-yl]methanamine,
(+)-1-[7-(2,6-dimethylphenyl)-2,3-dihydro-1-benzofuran-2-yl]methanamine,
(±)-1-[7-(2-methoxyphenyl)-2,3-dihydro-1-benzofuran-2-yl]methanamine,
(±)-1-[7-(2-chlorophenyl)-2,3-dihydro-1-benzofuran-2-yl]methanamine,
(±) 1-[7-(3-fluorophenyl)-2,3-dihydro-1-benzofuran-2-yl]methanamine,
(±)-1-[7-(3-chlorophenyl)-2,3-dihydro-1-benzofuran-2-yl]methanamine,
(+)-1-[7-(3-chlorophenyl)-2,3-dihydro-1-benzofuran-2-yl]methanamine,
(−)-1-[7-(3-chlorophenyl)-2,3-dihydro-1-benzofuran-2-yl]methanamine,
(±)-1-[7-(3-methoxyphenyl)-2,3-dihydro-1-benzofuran-2-yl]methanamine,
(±) 1-{7-[3-(trifluoromethyl)phenyl]-2,3-dihydro-1-benzofuran-2-yl}methanamine,
(±)-1-[7-(4-methylphenyl)-2,3-dihydro-1-benzofuran-2-yl]methanamine,
(+)-1-[7-(4-methylphenyl)-2,3-dihydro-1-benzofuran-2-yl]methanamine,
(−)-1-[7-(4-methylphenyl)-2,3-dihydro-1-benzofuran-2-yl]methanamine,
(±)-1-[7-(4-fluorophenyl)-2,3-dihydro-1-benzofuran-2-yl]methanamine,
(+)-1-[7-(4-fluorophenyl)-2,3-dihydro-1-benzofuran-2-yl]methanamine,
(−)-1-[7-(4-fluorophenyl)-2,3-dihydro-1-benzofuran-2-yl]methanamine,
(±)-1-[7-(4-chlorophenyl)-2,3-dihydro-1-benzofuran-2-yl]methanamine,
(+)-1-[7-(4-chlorophenyl)-2,3-dihydro-1-benzofuran-2-yl]methanamine,
(−)-1-[7-(4-chlorophenyl)-2,3-dihydro-1-benzofuran-2-yl]methanamine,
(±)-1-[7-(4-methoxyphenyl)-2,3-dihydro-1-benzofuran-2-yl]methanamine,
(+)-1-[7-(4-methoxyphenyl)-2,3-dihydro-1-benzofuran-2-yl]methanamine,
(−)-1-[7-(4-methoxyphenyl)-2,3-dihydro-1-benzofuran-2-yl]methanamine,
(±)-1-{7-[4-(trifluoromethyl)phenyl]-2,3-dihydro-1-benzofuran-2-yl}methanamine,
(±)-1-[7-(2,4-dichlorophenyl)-2,3-dihydro-1-benzofuran-2-yl]methanamine,
(±)-1-(5-chloro-7-phenyl-2,3-dihydro-1-benzofuran-2-yl)methanamine,
(+)-1-(5-chloro-7-phenyl-2,3-dihydro-1-benzofuran-2-yl)methanamine,
(−)-1-(5-chloro-7-phenyl-2,3-dihydro-1-benzofuran-2-yl)methanamine,
(±)-1-[5-chloro-7-(2-chlorophenyl)-2,3-dihydro-1-benzofuran-2-yl]methanamine,
(±)-1-[5-chloro-7-(3-methylphenyl)-2,3-dihydro-1-benzofuran-2-yl]methanamine,
(±)-1-[5-chloro-7-(3-chlorophenyl)-2,3-dihydro-1-benzofuran-2-yl]methanamine,
(±)-(5-chloro-7-thien-3-yl-2,3-dihydro-1-benzofuran-2-yl)methylamine,
(−)-(5-chloro-7-thien-3-yl-2,3-dihydro-1-benzofuran-2-yl)methylamine,
(+)-(5-chloro-7-thien-3-yl-2,3-dihydro-1-benzofuran-2-yl)methylamine,
(+)-N-[(5-chloro-7-thien-3-yl-2,3-dihydro-1-benzofuran-2-yl)methyl]-N-methylamine,
(−)-N-[(5-chloro-7-thien-3-yl-2,3-dihydro-1-benzofuran-2-yl)methyl]-N-methylamine,
(±)-1-[5-chloro-7-(2-methylphenyl)-2,3-dihydro-1-benzofuran-2-yl]methanamine,
(+)-1-[5-chloro-7-(2-methylphenyl)-2,3-dihydro-1-benzofuran-2-yl]methanamine,
(−)-1-[5-chloro-7-(2-methylphenyl)-2,3-dihydro-1-benzofuran-2-yl]methanamine,
(±)-1-(4-fluoro-7-phenyl-2,3-dihydro-1-benzofuran-2-yl)methanamine,
(±)-1-[4-fluoro-7-(2-methylphenyl)-2,3-dihydro-1-benzofuran-2-yl]methanamine,
(±)-1-[7-(2-chlorophenyl)-5-fluoro-2,3-dihydro-1-benzofuran-2-yl]methanamine,
(+)-1-[7-(2-chlorophenyl)-5-fluoro-2,3-dihydro-1-benzofuran-2-yl]methanamine,
(−)-1-[7-(2-chlorophenyl)-5-fluoro-2,3-dihydro-1-benzofuran-2-yl]methanamine,
(±)-1-(5-fluoro-7-phenyl-2,3-dihydro-1-benzofuran-2-yl)methanamine,
(±)-1-[5-fluoro-7-(2-methylphenyl)-2,3-dihydro-1-benzofuran-2-yl]methanamine,
(+)-1-[5-fluoro-7-(2-methylphenyl)-2,3-dihydro-1-benzofuran-2-yl]methanamine,
(−)-1-[5-fluoro-7-(2-methylphenyl)-2,3-dihydro-1-benzofuran-2-yl]methanamine,
(±)-1-[5-fluoro-7-(2-fluorophenyl)-2,3-dihydro-1-benzofuran-2-yl]methanamine,
(±)-1-{5-fluoro-7-[2-(trifluoromethyl)phenyl]-2,3-dihydro-1-benzofuran-2-yl}methanamine,
(±)-(4,5-difluoro-7-phenyl-2,3-dihydro-1-benzofuran-2-yl)methylamine,
(±)-1-[4,5-difluoro-7-(2-methylphenyl)-2,3-dihydro-1-benzofuran-2-yl]methanamine,
(±)-1-(5-chloro-2-methyl-7-phenyl-2,3-dihydro-1-benzofuran-2-yl)methanamine,
(±)-(5-chloro-2-methyl-7-thien-3-yl-2,3-dihydro-1-benzofuran-2-yl)methylamine,
(±)-(5-chloro-2-methyl-7-thien-2-yl-2,3-dihydro-1-benzofuran-2-yl)methylamine,
(+)-1-[7-(2-methoxyphenyl)-2,3-dihydro-1-benzofuran-2-yl]methanamine,
(−)-1-[7-(2-chlorophenyl)-2,3-dihydro-1-benzofuran-2-yl]methanamine,
(+)-1-[7-(2-chlorophenyl)-2,3-dihydro-1-benzofuran-2-yl]methanamine,
(+)-1-[7-(3-fluorophenyl)-2,3-dihydro-1-benzofuran-2-yl]methanamine,
(−)-1-[7-(3-fluorophenyl)-2,3-dihydro-1-benzofuran-2-yl]methanamine,
(+)-1-[7-(3-methoxyphenyl)-2,3-dihydro-1-benzofuran-2-yl]methanamine,
(−)-1-[7-(3-methoxyphenyl)-2,3-dihydro-1-benzofuran-2-yl]methanamine,
(−)-1-{7-[3-(trifluoromethyl)phenyl]-2,3-dihydro-1-benzofuran-2-yl}methanamine,
(+)-1-{7-[3-(trifluoromethyl)phenyl]-2,3-dihydro-1-benzofuran-2-yl}methanamine,
(+)-1-{7-[4-(trifluoromethyl)phenyl]-2,3-dihydro-1-benzofuran-2-yl}methanamine,
(−)-1-{7-[4-(trifluoromethyl)phenyl]-2,3-dihydro-1-benzofuran-2-yl}methanamine,
(±)-1-[7-(2,6-difluorophenyl)-2,3-dihydro-1-benzofuran-2-yl]methanamine,
(±)-1-[7-(2,6-dichlorophenyl)-2,3-dihydro-1-benzofuran-2-yl]methanamine,
(−)-1-[7-(2,6-dichlorophenyl)-2,3-dihydro-1-benzofuran-2-yl]methanamine,
(+)-1-[7-(2,6-dichlorophenyl)-2,3-dihydro-1-benzofuran-2-yl]methanamine,
(±)-1-[7-(2,4-dimethoxyphenyl)-2,3-dihydro-1-benzofuran-2-yl]methanamine,
(−)-1-[7-(2,4-dimethoxyphenyl)-2,3-dihydro-1-benzofuran-2-yl]methanamine,
(+)-1-[7-(2,4-dimethoxyphenyl)-2,3-dihydro-1-benzofuran-2-yl]methanamine,
(±)-1-[7-(2,4-difluorophenyl)-2,3-dihydro-1-benzofuran-2-yl]methanamine,
(+)-1-[7-(2,4-difluorophenyl)-2,3-dihydro-1-benzofuran-2-yl]methanamine,
(−)-1-[7-(2,4-difluorophenyl)-2,3-dihydro-1-benzofuran-2-yl]methanamine,
(−)-1-[7-(2,4-dichlorophenyl)-2,3-dihydro-1-benzofuran-2-yl]methanamine,
(+)-1-[7-(2,4-dichlorophenyl)-2,3-dihydro-1-benzofuran-2-yl]methanamine,
(−)-1-{5-fluoro-7-[2-(trifluoromethyl)phenyl]-2,3-dihydro-1-benzofuran-2-yl}methanamine,
(+)-1-{5-fluoro-7-[2-(trifluoromethyl)phenyl]-2,3-dihydro-1-benzofuran-2-yl}methanamine,
(±)-1-[7-(2,3-dimethylphenyl)-2,3-dihydro-1-benzofuran-2-yl]methanamine,
(±)-{[7-(2,3-dimethoxyphenyl)-2,3-dihydro-1-benzofuran-2-yl]methyl}amine,
(−)-{[(7-(2,3-dimethoxyphenyl)-2,3-dihydro-1-benzofuran-2-yl]methyl}amine,
(+)-{[(7-(2,3-dimethoxyphenyl)-2,3-dihydro-1-benzofuran-2-yl]methyl}amine,
(+)-{[7-(4-chloro-2-methylphenyl)-2,3-dihydro-1-benzofuran-2-yl]methyl}amine,
(−)-{[7-(4-chloro-2-methylphenyl)-2,3-dihydro-1-benzofuran-2-yl]methyl}amine,
(±)-{[7-(2,3-difluorophenyl)-2,3-dihydro-1-benzofuran-2-yl]methyl}amine,
(±)-{[7-(2,5-dimethylphenyl)-2,3-dihydro-1-benzofuran-2-yl]methyl}amine,
(±)-{[7-(2,5-dimethoxyphenyl)-2,3-dihydro-1-benzofuran-2-yl]methyl}amine,
(±)-{[7-(2,5-dichlorophenyl)-2,3-dihydro-1-benzofuran-2-yl]methyl}amine,
(+)-{[7-(2,5-dichlorophenyl)-2,3-dihydro-1-benzofuran-2-yl]methyl}amine,
(−)-{[7-(2,5-dichlorophenyl)-2,3-dihydro-1-benzofuran-yl]methyl}amine,
(±)-{[7-(2,4,6-trichlorophenyl)-2,3-dihydro-1-benzofuran-2-yl]methyl}amine,
(±)-{[7-(4-chloro-2-methylphenyl)-2,3-dihydro-1-benzofuran-2-yl]methyl}amine,
(±)-{[7-(5-chloro-2-methylphenyl)-2,3-dihydro-1-benzofuran-2-yl]methyl}amine,
(±)-{[7-(5-chloro-2-methoxyphenyl)-2,3-dihydro-1-benzofuran-2-yl]methyl}amine,
(±)-[(7-pyridin-3-yl-2,3-dihydro-1-benzofuran-2-yl)methyl]amine,
(+)-{[7-pyridin-3-yl-2,3-dihydro-1-benzofuran-2-yl]methyl}amine,
(−)-{[7-pyridin-3-yl-2,3-dihydro-1-benzofuran-2-yl]methyl}amine,
(±)-{[5-fluoro-7-(2-methoxyphenyl)-2,3-dihydro-1-benzofuran-2-yl]methyl}amine,
(±)-{[5-fluoro-7-(3-fluorophenyl)-2,3-dihydro-1-benzofuran-2-yl]methyl}amine,
(±)-{[5-fluoro-7-(3-methoxyphenyl)-2,3-dihydro-1-benzofuran-2-yl]methyl}amine,
(±)-{[5-fluoro-7-(3-methylphenyl)-2,3-dihydro-1-benzofuran-2-yl]methyl}amine,
(±)-{[5-fluoro-7-(4-fluorophenyl)-2,3-dihydro-1-benzofuran-2-yl]methyl}amine,
(±)-{[5-fluoro-7-(4-chlorophenyl)-2,3-dihydro-1-benzofuran-2-yl]methyl}amine,
(±)-{[5-fluoro-7-(4-methylphenyl)-2,3-dihydro-1-benzofuran-2-yl]methyl}amine,
(±)-{[5-fluoro-7-(4-methoxyphenyl)-2,3-dihydro-1-benzofuran-2-yl]methyl}amine,
(±)-[(5-fluoro-7-thien-3-yl-2,3-dihydro-1-benzofuran-2-yl)methyl]amine,
(±)-{[5-fluoro-7-(3-furyl)-2,3-dihydro-1-benzofuran-2-yl]methyl}amine,
(±)-[(5-fluoro-7-pyridin-2-yl-2,3-dihydro-1-benzofuran-2-yl)methyl]amine,
(±)-[(5-fluoro-7-pyridin-3-yl-2,3-dihydro-1-benzofuran-2-yl)methyl]amine,
(−)-[(5-fluoro-7-pyridin-3-yl-2,3-dihydro-1-benzofuran-2-yl)methyl]amine,
(+)-{[5-fluoro-7-pyridin-3-yl-2,3-dihydro-1-benzofuran-2-yl]methyl}amine,
(±)-[(5-fluoro-7-pyridin-4-yl-2,3-dihydro-1-benzofuran-2-yl)methyl]amine,
(±)-[(5-fluoro-7-pyrimidin-5-yl-2,3-dihydro-1-benzofuran-2-yl)methyl]amine,
(±)-{[7-(2,3-dichlorophenyl)-5-fluoro-2,3-dihydro-1-benzofuran-2-yl]methyl}amine,
(±)-{[7-(2,3-dimethoxyphenyl)-5-fluoro-2,3-dihydro-1-benzofuran-2-yl]methyl}amine,
(−)-{[7-(2,3-dimethoxyphenyl)-5-fluoro-2,3-dihydro-1-benzofuran-2-yl]methyl}amine,
(+)-{[7-(2,3-dimethoxyphenyl)-5-fluoro-2,3-dihydro-1-benzofuran-2-yl]methyl}amine,
(±)-{[7-(2,4-difluorophenyl)-5-fluoro-2,3-dihydro-1-benzofuran-2-yl]methyl}amine,
(±)-{[7-(2,4-dichlorophenyl)-5-fluoro-2,3-dihydro-1-benzofuran-2-yl]methyl}amine,
(−)-{[5-fluoro-7-(2,4-dichlorophenyl)-2,3-dihydro-1-benzofuran-2-yl]methyl}amine,
(+)-{[5-fluoro-7-(2,4-dichlorophenyl)-2,3-dihydro-1-benzofuran-2-yl]methyl}amine,
(±)-{[7-(2,4-dimethoxyphenyl)-5-fluoro-2,3-dihydro-1-benzofuran-2-yl]methyl}amine,
(±)-{[7-(2,5-difluorophenyl)-5-fluoro-2,3-dihydro-1-benzofuran-2-yl]methyl}amine,
(±)-{[7-(2,5-dichlorophenyl)-5-fluoro-2,3-dihydro-1-benzofuran-2-yl]methyl}amine,
(±)-{[7-(2,5-dimethylphenyl)-5-fluoro-2,3-dihydro-1-benzofuran-2-yl]methyl}amine,
(±)-{[7-(2,5-dimethoxyphenyl)-5-fluoro-2,3-dihydro-1-benzofuran-2-yl]methyl}amine,
(±)-{[5-fluoro-7-(5-methoxy-2-methylphenyl)-2,3-dihydro-1-benzofuran-2-yl]methyl}amine,
(±)-{[5-fluoro-7-(2-methoxy-5-methylphenyl)-2,3-dihydro-1-benzofuran-2-yl]methyl}amine,
(±)-{[7-(2,6-difluorophenyl)-5-fluoro-2,3-dihydro-1-benzofuran-2-yl]methyl}amine,
(±)-{[7-(2,6-dimethylphenyl)-5-fluoro-2,3-dihydro-1-benzofuran-2-yl]methyl}amine,
(+)-{[7-(2,6-dimethylphenyl)-5-fluoro-2,3-dihydro-1-benzofuran-2-yl]methyl}amine,
(−)-{[7-(2,6-dimethylphenyl)-5-fluoro-2,3-dihydro-1-benzofuran-2-yl]methyl}amine,
(±)-{[7-(2,6-dichlorophenyl)-5-fluoro-2,3-dihydro-1-benzofuran-2-yl]methyl}amine,
(±)-N-{[7-(2,6-dichlorophenyl)-5-fluoro-2,3-dihydro-1-benzofuran-2-yl]methyl}cyclopropanamine,
(±)-1-cyclopropyl-N-{[7-(2,6-dichlorophenyl)-5-fluoro-2,3-dihydro-1-benzofuran-2-yl]methyl}methanamine,
(±)-N-{[7-(2,6-dichlorophenyl)-5-fluoro-2,3-dihydro-1-benzofuran-2-yl]methyl}ethanamine,
(±)-{[7-(2,6-dichlorophenyl)-5-fluoro-2,3-dihydro-1-benzofuran-2-yl]methyl}dimethylamine,
(±)-{[5-chloro-7-(2-fluorophenyl)-2,3-dihydro-1-benzofuran-2-yl]methyl}amine,
(±)-{[5-chloro-7-(2-methoxyphenyl)-2,3-dihydro-1-benzofuran-2-yl]methyl}amine,
(±)-{[5-chloro-7-(3-furyl)-2,3-dihydro-1-benzofuran-2-yl]methyl}amine,
(±)-{[5-chloro-7-(2,3-difluorophenyl)-2,3-dihydro-1-benzofuran-2-yl]methyl}amine,
(−)-{[5-chloro-7-(2,3-difluorophenyl)-2,3-dihydro-1-benzofuran-2-yl]methyl}amine,
(+)-{[5-chloro-7-(2,3-difluorophenyl)-2,3-dihydro-1-benzofuran-2-yl]methyl}amine,
(±)-{[5-chloro-7-(2,3-dichlorophenyl)-2,3-dihydro-1-benzofuran-2-yl]methyl}amine,
(±)-{[5-chloro-7-(2,3-dimethylphenyl)-2,3-dihydro-1-benzofuran-2-yl]methyl}amine,
(±)-{[5-chloro-7-(2,3-dimethoxyphenyl)-2,3-dihydro-1-benzofuran-2-yl]methyl}amine,
(±)-{[5-chloro-7-(2,4-difluorophenyl)-2,3-dihydro-1-benzofuran-2-yl]methyl}amine,
(±)-{[5-chloro-7-(2,4-dichlorophenyl)-2,3-dihydro-1-benzofuran-2-yl]methyl}amine,
(−)-{[5-chloro-7-(2,4-dichlorophenyl)-2,3-dihydro-1-benzofuran-2-yl]methyl}amine,
(+)-{[5-chloro-7-(2,4-dichlorophenyl)-2,3-dihydro-1-benzofuran-2-yl]methyl}amine,
(±)-{[5-chloro-7-(2,4-dimethoxyphenyl)-2,3-dihydro-1-benzofuran-2-yl]methyl}amine,
(±)-{[5-chloro-7-(2,5-difluorophenyl)-2,3-dihydro-1-benzofuran-2-yl]methyl}amine,
(±) {[5-chloro-7-(2,5-dichlorophenyl)-2,3-dihydro-1-benzofuran-2-yl]methyl}amine,
(±)-{[5-chloro-7-(5-chloro-2-methoxyphenyl)-2,3-dihydro-1-benzofuran-2-yl]methyl}amine,
(±)-{[5-chloro-7-(3,4-difluorophenyl)-2,3-dihydro-1-benzofuran-2-yl]methyl}amine,
(±)-{[5-chloro-7-(3-chloro-4-fluorophenyl)-2,3-dihydro-1-benzofuran-2-yl]methyl}amine,
(±)-{[5-chloro-7-(2,6-dimethylphenyl)-2,3-dihydro-1-benzofuran-2-yl]methyl}amine,
(−)-{[5-chloro-7-(2,6-dimethylphenyl)-2,3-dihydro-1-benzofuran-2-yl]methyl}amine,
(+)-{[5-chloro-7-(2,6-dimethylphenyl)-2,3-dihydro-1-benzofuran-2-yl]methyl}amine,
(±)-{[5-chloro-7-(2,6-dichlorophenyl)-2,3-dihydro-1-benzofuran-2-yl]methyl}amine,
(+)-{[5-chloro-7-(2,6-dichlorophenyl)-2,3-dihydro-1-benzofuran-2-yl]methyl}amine,
(−)-{[5-chloro-7-(2,6-dichlorophenyl)-2,3-dihydro-1-benzofuran-2-yl]methyl}amine,
(±)-{[(5-chloro-7-pyridin-3-yl-2,3-dihydro-1-benzofuran-2-yl)methyl]amine,
(±)-N-{[5-chloro-7-(2,6-dimethylphenyl)-2,3-dihydro-1-benzofuran-2-yl]methyl}cyclopropanamine,
(±)-N-{[5-chloro-7-(2,6-dimethylphenyl)-2,3-dihydro-1-benzofuran-2-yl]methyl}(cyclopropylmethyl)amine,
(±)-N-{[5-chloro-7-(2,6-dimethylphenyl)-2,3-dihydro-1-benzofuran-2-yl]methyl}ethanamine,
(±)-{[(5-methyl-7-phenyl-2,3-dihydro-1-benzofuran-2-yl)methyl]amine,
(±)-{[7-(2-methylphenyl)-5-methyl-2,3-dihydro-1-benzofuran-2-yl]methyl}amine,
(±)-{[7-(2-fluorophenyl)-5-methyl-2,3-dihydro-1-benzofuran-2-yl]methyl}amine,
(±)-{[7-(2-methoxyphenyl)-5-methyl-2,3-dihydro-1-benzofuran-2-yl]methyl}amine,
(±)-{[7-(2-chlorophenyl)-5-methyl-2,3-dihydro-1-benzofuran-2-yl]methyl}amine,
(±)-({5-methyl-7-[2-(trifluoromethyl)phenyl]-2,3-dihydro-1-benzofuran-2-yl}methyl)amine,
(±)-{[7-(3-chlorophenyl)-5-methyl-2,3-dihydro-1-benzofuran-2-yl]methyl}amine,
(±)-{[7-(3-methylphenyl)-5-methyl-2,3-dihydro-1-benzofuran-2-yl]methyl}amine,
(±)-{[7-(4-methylphenyl)-5-methyl-2,3-dihydro-1-benzofuran-2-yl]methyl}amine,
(±) {[7-(4-fluorophenyl)-5-methyl-2,3-dihydro-1-benzofuran-2-yl]methyl}amine,
(±)-{[7-(4-chlorophenyl)-5-methyl-2,3-dihydro-1-benzofuran-2-yl]methyl}amine,
(±)-{[7-(4-methoxyphenyl)-5-methyl-2,3-dihydro-1-benzofuran-2-yl]methyl}amine,
(±)-{[7-(2,3-dimethoxyphenyl)-5-methyl-2,3-dihydro-1-benzofuran-2-yl]methyl}amine,
(−)-{[7-(2,3-dimethoxyphenyl)-5-methyl-2,3-dihydro-1-benzofuran-2-yl]methyl}amine,
(+)-{[7-(2,3-dimethoxyphenyl)-5-methyl-2,3-dihydro-1-benzofuran-2-yl]methyl}amine,
(±)-{[7-(2,4-dichlorophenyl)-5-methyl-2,3-dihydro-1-benzofuran-2-yl]methyl}amine,
(−)-{[7-(2,4-dichlorophenyl)-5-methyl-2,3-dihydro-1-benzofuran-2-yl]methyl}amine,
(+)-{[7-(2,4-dichlorophenyl)-5-methyl-2,3-dihydro-1-benzofuran-2-yl]methyl}amine,
(±)-{[7-(2,5-dichlorophenyl)-5-methyl-2,3-dihydro-1-benzofuran-2-yl]methyl}amine,
(±)-{[7-(2,6-dimethylphenyl)-5-methyl-2,3-dihydro-1-benzofuran-2-yl]methyl}amine,
(±)-{[7-(2,6-dichlorophenyl)-5-methyl-2,3-dihydro-1-benzofuran-2-yl]methyl}amine,
(−)-{[7-(2,6-dichlorophenyl)-5-methyl-2,3-dihydro-1-benzofuran-2-yl]methyl}amine,
(+)-{[7-(2,6-dichlorophenyl)-5-methyl-2,3-dihydro-1-benzofuran-2-yl]methyl}amine,
(±)-{[5-ethyl-7-(2-methylphenyl)-2,3-dihydro-1-benzofuran-2-yl]methyl}amine,
(±)-{[5-ethyl-7-(2-chlorophenyl)-2,3-dihydro-1-benzofuran-2-yl]methyl}amine,
(±)-{[5-(trifluoromethyl)-7-(2-fluorophenyl)-2,3-dihydro-1-benzofuran-2-yl]methyl}amine,
(±)-{[5-(trifluoromethyl)-7-(2-methylphenyl)-2,3-dihydro-1-benzofuran-2-yl]methyl}amine,
(±)-{[5-(trifluoromethyl)-7-(2-chlorophenyl)-2,3-dihydro-1-benzofuran-2-yl]methyl}amine,
(±)-{[5-(trifluoromethyl)-7-(2-methoxyphenyl)-2,3-dihydro-1-benzofuran-2-yl]methyl}amine,
(±)-{[5-(trifluoromethyl)-7-(2-(trifluoromethyl)phenyl)-2,3-dihydro-1-benzofuran-2-yl]methyl}amine,
(±)-{[5-(trifluoromethyl)-7-(3-methylphenyl)-2,3-dihydro-1-benzofuran-2-yl]methyl}amine,
(±)-{[5-(trifluoromethyl)-7-(3-fluorophenyl)-2,3-dihydro-1-benzofuran-2-yl]methyl}amine,
(±)-{[5-(trifluoromethyl)-7-(3-chlorophenyl)-2,3-dihydro-1-benzofuran-2-yl]methyl}amine,
(±)-{[5-(trifluoromethyl)-7-(3-methoxyphenyl)-2,3-dihydro-1-benzofuran-2-yl]methyl}amine,
(±)-{[5-(trifluoromethyl)-7-(3-(trifluoromethyl)phenyl)-2,3-dihydro-1-benzofuran-2-yl]methyl}amine,
(±)-{[5-(trifluoromethyl)-7-(4-methylphenyl)-2,3-dihydro-1-benzofuran-2-yl]methyl}amine,
(±)-{[5-(trifluoromethyl)-7-(4-fluorophenyl)-2,3-dihydro-1-benzofuran-2-yl]methyl}amine,
(±)-{[5-(trifluoromethyl)-7-(4-chlorophenyl)-2,3-dihydro-1-benzofuran-2-yl]methyl}amine,
(±)-{[5-(trifluoromethyl)-7-(4-methoxyphenyl)-2,3-dihydro-1-benzofuran-2-yl]methyl}amine,
(±)-{[5-(trifluoromethyl)-7-(4-(trifluoromethyl)phenyl)-2,3-dihydro-1-benzofuran-2-yl]methyl}amine,
(±)-{[7-(2,3-dimethylphenyl)-5-(trifluoromethyl)-2,3-dihydro-1-benzofuran-2-yl]methyl}amine,
(±)-{[7-(2,3-difluorophenyl)-5-(trifluoromethyl)-2,3-dihydro-1-benzofuran-2-yl]methyl}amine,
(±)-{[7-(2,3-dichlorophenyl)-5-(trifluoromethyl)-2,3-dihydro-1-benzofuran-2-yl]methyl) amine,
(±)-{[7-(2,3-dimethoxyphenyl)-5-(trifluoromethyl)-2,3-dihydro-1-benzofuran-2-yl]methyl}amine,
(±)-{[7-(2,4-difluorophenyl)-5-(trifluoromethyl)-2,3-dihydro-1-benzofuran-2-yl]methyl}amine,
(±)-{[7-(2,4-dimethoxyphenyl)-5-(trifluoromethyl)-2,3-dihydro-1-benzofuran-2-yl]methyl}amine,
(±)-{[7-(3,4-difluorophenyl)-5-(trifluoromethyl)-2,3-dihydro-1-benzofuran-2-yl]methyl}amine,
(±)-{[7-(3-chloro-4-fluorophenyl)-5-(trifluoromethyl)-2,3-dihydro-1-benzofuran-2-yl]methyl}amine,
(±)-{[7-(2,5-difluorophenyl)-5-(trifluoromethyl)-2,3-dihydro-1-benzofuran-2-yl]methyl}amine,
(±)-{[7-(2,5-dichlorophenyl)-5-(trifluoromethyl)-2,3-dihydro-1-benzofuran-2-yl]methyl}amine,
(±)-{[7-(2,6-dimethylphenyl)-5-(trifluoromethyl)-2,3-dihydro-1-benzofuran-2-yl]methyl}amine,
(±)-4-[2-(aminomethyl)-5-(trifluoromethyl)-2,3-dihydro-1-benzofuran-7-yl]benzonitrile
(±)-{[7-(3-furyl)-5-(trifluoromethyl)-2,3-dihydro-1-benzofuran-2-yl]methyl}amine,
(±)-{[7-thien-3-yl-5-(trifluoromethyl)-2,3-dihydro-1-benzofuran-2-yl]methyl}amine,
(±)-{[7-pyridin-3-yl-5-(trifluoromethyl)-2,3-dihydro-1-benzofuran-2-yl]methyl}amine,
(±)-{[7-(2-fluorophenyl)-5-methoxy-2,3-dihydro-1-benzofuran-2-yl]methyl}amine,
(±)-{[7-(2-chlorophenyl)-5-methoxy-2,3-dihydro-1-benzofuran-2-yl]methyl}amine,
(±)-{[7-(2-methylphenyl)-5-methoxy-2,3-dihydro-1-benzofuran-2-yl]methyl}amine,
(±)-{[7-(2-methoxyphenyl)-5-methoxy-2,3-dihydro-1-benzofuran-2-yl]methyl}amine,
(±)-{[5-methoxy-7-(3-thienyl)-2,3-dihydro-1-benzofuran-2-yl]methyl}amine,
(±)-{[7-(2,3-difluorophenyl)-5-methoxy-2,3-dihydro-1-benzofuran-2-yl]methyl}amine,
(±)-{[7-(2,3-dichlorophenyl)-5-methoxy-2,3-dihydro-1-benzofuran-2-yl]methyl}amine,
(±)-{[7-(2,3-dimethylphenyl)-5-methoxy-2,3-dihydro-1-benzofuran-2-yl]methyl}amine,
(±)-{[7-(2,4-difluorophenyl)-5-methoxy-2,3-dihydro-1-benzofuran-2-yl]methyl}amine,
(±)-{[7-(2,4-dichlorophenyl)-5-methoxy-2,3-dihydro-1-benzofuran-2-yl]methyl}amine,
(±)-{[7-(2,5-difluorophenyl)-5-methoxy-2,3-dihydro-1-benzofuran-2-yl]methyl}amine,
(±)-{[7-(2,5-dichlorophenyl)-5-methoxy-2,3-dihydro-1-benzofuran-2-yl]methyl}amine,
(+)-{[7-(2,5-dichlorophenyl)-5-methoxy-2,3-dihydro-1-benzofuran-2-yl]methyl}amine,
(−)-{[7-(2,5-dichlorophenyl)-5-methoxy-2,3-dihydro-1-benzofuran-2-yl]methyl}amine,
(±)-{[7-(2,5-dimethylphenyl)-5-methoxy-2,3-dihydro-1-benzofuran-2-yl]methyl}amine,
(±)-{[7-(2,5-dimethoxylphenyl)-5-methoxy-2,3-dihydro-1-benzofuran-2-yl]methyl}amine,
(±)-{[7-(5-chloro-2-methoxyphenyl)-5-methoxy-2,3-dihydro-1-benzofuran-2-yl]methyl}amine,
(±)-{[7-(3-chloro-4-fluorophenyl)-5-methoxy-2,3-dihydro-1-benzofuran-2-yl]methyl}amine,
(±)-{[7-(2,6-dimethylphenyl)-5-methoxy-2,3-dihydro-1-benzofuran-2-yl]methyl}amine,
(±)-[(N-methyl-1-[7-(2-methoxyphenyl)-2,3-dihydro-1-benzofuran-2-yl]methanamine,
(±)-[(N-methyl-1-[7-(3-methylphenyl)-2,3-dihydro-1-benzofuran-2-yl]methanamine,
(±)-[(N-methyl-1-[7-(3-fluorophenyl)-2,3-dihydro-1-benzofuran-2-yl]methanamine,
(±)-[(N-methyl-1-[7-(3-chlorophenyl)-2,3-dihydro-1-benzofuran-2-yl]methanamine,
(±)-[(N-methyl-1-[7-(3-methoxyphenyl)-2,3-dihydro-1-benzofuran-2-yl]methanamine,
(±)-[(N-methyl-1-[7-(4-methylphenyl)-2,3-dihydro-1-benzofuran-2-yl]methanamine,
(±)-[(N-methyl-1-[7-(4-fluorophenyl)-2,3-dihydro-1-benzofuran-2-yl]methanamine,
(±)-[(N-methyl-1-[7-(4-chlorophenyl)-2,3-dihydro-1-benzofuran-2-yl]methanamine,
(±)-[(N-methyl-1-[7-(4-methoxyphenyl)-2,3-dihydro-1-benzofuran-2-yl]methanamine,
(±)-[(N-methyl-1-[7-(2,3-dimethylphenyl)-2,3-dihydro-1-benzofuran-2-yl]methanamine,
(±)-[(N-methyl-1-[7-(2,3-dimethoxyphenyl)-2,3-dihydro-1-benzofuran-2-yl]methanamine,
(±)-[(N-methyl-1-[7-(2,4-difluorophenyl)-2,3-dihydro-1-benzofuran-2-yl]methanamine,
(±)-[(N-methyl-1-[7-(2,4-dichlorophenyl)-2,3-dihydro-1-benzofuran-2-yl]methanamine,
(±)-[(N-methyl-1-[7-(2,4-dimethoxyphenyl)-2,3-dihydro-1-benzofuran-2-yl]methanamine,
(±)-[(N-methyl-1-[7-(2,5-dimethylphenyl)-2,3-dihydro-1-benzofuran-2-yl]methanamine,
(±)-[(N-methyl-1-[7-(2,5-difluorophenyl)-2,3-dihydro-1-benzofuran-2-yl]methanamine,
(±)-[(N-methyl-1-[7-(2,5-dichlorophenyl)-2,3-dihydro-1-benzofuran-2-yl]methanamine,
(±)-[(N-methyl-1-[7-(5-chloro-2-methoxyphenyl)-2,3-dihydro-1-benzofuran-2-yl]methanamine,
(±)-[(N-methyl-1-[7-(5-chloro-2-methylphenyl)-2,3-dihydro-1-benzofuran-2-yl]methanamine,
(±)-[(N-methyl-1-[7-(2,6-dimethylphenyl)-2,3-dihydro-1-benzofuran-2-yl]methanamine,
(±)-[(N-methyl-1-[7-(2,6-dichlorophenyl)-2,3-dihydro-1-benzofuran-2-yl]methanamine,
(−)-{[7-(2,6-dichlorophenyl)-2,3-dihydro-1-benzofuran-2-yl]methyl}methylamine,
(+)-{[7-(2,6-dichlorophenyl)-2,3-dihydro-1-benzofuran-2-yl]methyl}methylamine,
(±)-N-methyl-1-(7-pyridin-3-yl-2,3-dihydro-1-benzofuran-2-yl)methanamine,
(±)-[(N-methyl-1-[7-(2,3-difluorophenyl)-2,3-dihydro-1-benzofuran-2-yl]methanamine,
(±)-{[5-fluoro-7-(2-fluorophenyl)-2,3-dihydro-1-benzofuran-2-yl]methyl}methylamine,
(±)-{[5-fluoro-7-(2-chlorophenyl)-2,3-dihydro-1-benzofuran-2-yl]methyl}methylamine,
(±)-{[5-fluoro-7-(2-methylphenyl)-2,3-dihydro-1-benzofuran-2-yl]methyl}methylamine,
(−)-{[5-fluoro-7-(2-methylphenyl)-2,3-dihydro-1-benzofuran-2-yl]methyl}methylamine,
(+)-{[5-fluoro-7-(2-methylphenyl)-2,3-dihydro-1-benzofuran-2-yl]methyl}methylamine,
(±)-{[5-fluoro-7-(2-methoxyphenyl)-2,3-dihydro-1-benzofuran-2-yl]methyl}methylamine,
(±)-{[7-(2,3-dimethoxyphenyl)-5-fluoro-2,3-dihydro-1-benzofuran-2-yl]methyl}methylamine,
(±)-[7-(2,4-dichlorophenyl)-5-fluoro-2,3-dihydro-1-benzofuran-2-yl]methyl}methylamine,
(−)-[7-(2,4-dichlorophenyl)-5-fluoro-2,3-dihydro-1-benzofuran-2-yl]methyl}methylamine,
(+)-[7-(2,4-dichlorophenyl)-5-fluoro-2,3-dihydro-1-benzofuran-2-yl]methyl}methylamine,
(±)-{[7-(2,5-difluorophenyl)-5-fluoro-2,3-dihydro-1-benzofuran-2-yl]methyl}methylamine,
(±)-{[7-(2,5-dichlorophenyl)-5-fluoro-2,3-dihydro-1-benzofuran-2-yl]methyl}methylamine,
(+)-{[5-fluoro-7-(2,5-dichlorophenyl)-2,3-dihydro-1-benzofuran-2-yl]methyl}methylamine,
(−)-{[5-fluoro-7-(2,5-dichlorophenyl)-2,3-dihydro-1-benzofuran-2-yl]methyl}methylamine,
(±)-{[7-(2,5-dimethylphenyl)-5-fluoro-2,3-dihydro-1-benzofuran-2-yl]methyl}methylamine,
(±)-{[7-(2,6-dimethylphenyl)-5-fluoro-2,3-dihydro-1-benzofuran-2-yl]methyl}methylamine,
(±)-{[7-(2,6-dichlorophenyl)-5-fluoro-2,3-dihydro-1-benzofuran-2-yl]methyl}methylamine,
(±)-{[5-fluoro-7-(5-methoxy-2-methylphenyl)-2,3-dihydro-1-benzofuran-2-yl]methyl}methylamine,
(±)-{[5-fluoro-7-(2-methoxy-5-methylphenyl)-2,3-dihydro-1-benzofuran-2-yl]methyl}methylamine,
(±)-{[7-(5-chloro-2-methoxyphenyl)-5-fluoro-2,3-dihydro-1-benzofuran-2-yl]methyl}methylamine,
(±)-[(5-fluoro-7-pyridin-3-yl-2,3-dihydro-1-benzofuran-2-yl)methyl]methylamine,
(±)-[(5-chloro-7-(2-methoxyphenyl)-2,3-dihydro-1-benzofuran-2-yl]methyl}methylamine,
(±)-{[5-chloro-7-(2,6-dimethylphenyl)-2,3-dihydro-1-benzofuran-2-yl]methyl}methylamine,
(±)-[(5-chloro-7-(2-fluorophenyl)-2,3-dihydro-1-benzofuran-2-yl]methyl}methylamine,
(±)-{[5-chloro-7-(2,3-difluorophenyl)-2,3-dihydro-1-benzofuran-2-yl]methyl}methylamine,
(±)-{[5-chloro-7-(2,3-dichlorophenyl)-2,3-dihydro-1-benzofuran-2-yl]methyl}methylamine,
(±)-{[5-chloro-7-(2,3-dimethylphenyl)-2,3-dihydro-1-benzofuran-2-yl]methyl}methylamine,
(±)-{[5-chloro-7-(2,3-dimethoxyphenyl)-2,3-dihydro-1-benzofuran-2-yl]methyl)methylamine,
(±)-{[5-chloro-7-(2,4-difluorophenyl)-2,3-dihydro-1-benzofuran-2-yl]methyl}methylamine,
(±)-{[5-chloro-7-(2,4-dichlorophenyl)-2,3-dihydro-1-benzofuran-2-yl]methyl}methylamine,
(±)-{[5-chloro-7-(2,4-dimethoxyphenyl)-2,3-dihydro-1-benzofuran-2-yl]methyl}methylamine,
(±)-{[5-chloro-7-(2,5-difluorophenyl)-2,3-dihydro-1-benzofuran-2-yl]methyl}methylamine,
(±)-{[5-chloro-7-(2,5-dichlorophenyl)-2,3-dihydro-1-benzofuran-2-yl]methyl}methylamine,
(±)-{[5-chloro-7-(5-chloro-2-methoxyphenyl)-2,3-dihydro-1-benzofuran-2-yl]methyl}methylamine,
(±)-{[5-chloro-7-(3.4-difluorophenyl)-2,3-dihydro-1-benzofuran-2-yl]methyl}methylamine,
(±)-[5-chloro-7-(3-chloro-4-fluorophenyl)-2,3-dihydro-1-benzofuran-2-yl]methyl}methylamine,
(±)-{[7-(2-fluorophenyl)-5-methyl-2,3-dihydro-1-benzofuran-2-yl]methyl}methylamine,
(±)-{[7-(2-chlorophenyl)-5-methyl-2,3-dihydro-1-benzofuran-2-yl]methyl}methylamine,
(±)-{[7-(2-methoxyphenyl)-5-methyl-2,3-dihydro-1-benzofuran-2-yl]methyl}methylamine,
(±)-{[7-(3-methylphenyl)-5-methyl-2,3-dihydro-1-benzofuran-2-yl]methyl}methylamine,
(±)-{[7-(3-chlorophenyl)-5-methyl-2,3-dihydro-1-benzofuran-2-yl]methyl}methylamine,
(±)-{[7-(4-methylphenyl)-5-methyl-2,3-dihydro-1-benzofuran-2-yl]methyl}methylamine,
(±)-{[7-(4-chlorophenyl)-5-methyl-2,3-dihydro-1-benzofuran-2-yl]methyl}methylamine,
(±)-{[7-(4-fluorophenyl)-5-methyl-2,3-dihydro-1-benzofuran-2-yl]methyl}methylamine,
(±)-{[7-(4-methoxyphenyl)-5-methyl-2,3-dihydro-1-benzofuran-2-yl]methyl}methylamine,
(±)-{[7-(2,3-dimethoxyphenyl)-5-methyl-2,3-dihydro-1-benzofuran-2-yl]methyl}methylamine,
(±)-{[7-(2,4-dichlorophenyl)-5-methyl-2,3-dihydro-1-benzofuran-2-yl]methyl}methylamine,
(±)-{[7-(2,5-dichlorophenyl)-5-methyl-2,3-dihydro-1-benzofuran-2-yl]methyl}methylamine,
(+)-{[7-(2,5-dichlorophenyl)-5-methyl-2,3-dihydro-1-benzofuran-2-yl]methyl}methylamine,
(−)-{[7-(2,5-dichlorophenyl)-5-methyl-2,3-dihydro-1-benzofuran-2-yl]methyl}methylamine,
(±)-{[7-(2,6-dimethylphenyl)-5-methyl-2,3-dihydro-1-benzofuran-2-yl]methyl}methylamine,
(±)-{[7-(2,6-dichlorophenyl)-5-methyl-2,3-dihydro-1-benzofuran-2-yl]methyl}methylamine,
(±)-{[7-(2-fluorophenyl)-5-methoxy-2,3-dihydro-1-benzofuran-2-yl]methyl}methylamine,
(±)-{[7-(2-chlorophenyl)-5-methoxy-2,3-dihydro-1-benzofuran-2-yl]methyl}methylamine,
(±)-{[7-(2-methylphenyl)-5-methoxy-2,3-dihydro-1-benzofuran-2-yl]methyl}methylamine,
(±)-{[7-(2,3-difluorophenyl)-5-methoxy-2,3-dihydro-1-benzofuran-2-yl]methyl}methylamine,
(±)-{[7-(2,3-dichlorophenyl)-5-methoxy-2,3-dihydro-1-benzofuran-2-yl]methyl}methylamine,
(±)-{[7-(2,3-dimethylphenyl)-5-methoxy-2,3-dihydro-1-benzofuran-2-yl]methyl}methylamine,
(±)-{[7-(2,4-difluorophenyl)-5-methoxy-2,3-dihydro-1-benzofuran-2-yl]methyl}methylamine,
(±)-{[7-(2,5-difluorophenyl)-5-methoxy-2,3-dihydro-1-benzofuran-2-yl]methyl}methylamine,
(±)-{[7-(2,5-dichlorophenyl)-5-methoxy-2,3-dihydro-1-benzofuran-2-yl]methyl}methylamine,
(±)-{[7-(2,5-dimethylphenyl)-5-methoxy-2,3-dihydro-1-benzofuran-2-yl]methyl}methylamine,
(±)-{[7-(2,5-dimethoxyphenyl)-5-methoxy-2,3-dihydro-1-benzofuran-2-yl]methyl}methylamine,
(±)-{[7-(5-chloro-2-methoxyphenyl)-5-methoxy-2,3-dihydro-1-benzofuran-2-yl]methyl}methylamine,
(±)-{[7-(2,6-dimethylphenyl)-5-methoxy-2,3-dihydro-1-benzofuran-2-yl]methyl}methylamine,
(±)-N-methyl-1-[7-(2,3-difluorophenyl)-5-(trifluoromethyl)-2,3-dihydro-1-benzofuran-2-yl]methanamine,
(±)-N-methyl-1-[7-(3,4-difluorophenyl)-5-(trifluoromethyl)-2,3-dihydro-1-benzofuran-2-yl]methanamine,
(±)-N-methyl-1-[7-(2,5-difluorophenyl)-5-(trifluoromethyl)-2,3-dihydro-1-benzofuran-2-yl]methanamine,
(±)-N-methyl-1-[7-(2,3-dichlorophenyl)-5-(trifluoromethyl)-2,3-dihydro-1-benzofuran-2-yl]methanamine,
(±) {[7-(3-chloro-4-fluorophenyl)-5-(trifluoromethyl)-2,3-dihydro-1-benzofuran-2-yl]methyl}methylamine,
(±)-N-methyl-1-[7-(2,4-dimethoxyphenyl)-5-(trifluoromethyl)-2,3-dihydro-1-benzofuran-2-yl]methanamine,
(+){[7-(2,6-dichlorophenyl)-5-fluoro-2,3-dihydro-1-benzofuran-2-yl]methyl}methylamine,
(−) {[7-(2,6-dichlorophenyl)-5-fluoro-2,3-dihydro-1-benzofuran-2-yl]methyl}methylamine,
(R)-[7-(2-chloro-phenyl)-(5-Fluoro-2,3-dihydro-benzofuran-2-ylmethyl)methyl-amine,
(R)-[7-(2,6-dichloro-phenyl)-5-Fluoro-2,3-dihydro-benzofuran-2-ylmethyl]ethylamine,
(R)-[7-(2,6-dichloro-phenyl)-5-Fluoro-2,3-dihydro-benzofuran-2-ylmethyl]dimethylamine,
{[(2R)-7-(5-chloro-2-methylphenyl)-5-fluoro-2,3-dihydro-1-benzofuran-2-yl]methyl}amine,
{[(2R)-7-(4-chloro-2-methylphenyl)-5-fluoro-2,3-dihydro-1-benzofuran-2-yl]methyl}amine,
(−)-{[7-(2,6-dichlorophenyl)-5-fluoro-2,3-dihydro-1-benzofuran-2-yl]methyl}amine,
(+)-{[7-(2,6 dichlorophenyl)-5-fluoro-2,3-dihydro-1-benzofuran-2-yl]methyl}amine,
(±)-{2-[6-chloro-7-(2-chlorophenyl)-2,3-dihydro-1-benzofuran-2-yl]ethyl}amine,
(±)-{2-[7-(2,6-dichlorophenyl)-5-fluoro-2,3-dihydro-1-benzofuran-2-yl]ethyl}amine,
(±)-{2-[7-(2-methoxyphenyl)-5-methoxy-2,3-dihydro-1-benzofuran-2-yl]ethyl}amine,
(±)-{N-methyl-1-[(7-(2,4,6-trichlorophenyl)-2,3-dihydro-1-benzofuran-2-yl]methanamine,
(+)-{N-methyl-1-[(7-(2,4,6-trichlorophenyl)-2,3-dihydro-1-benzofuran-2-yl]methanamine,
(−)-{N-methyl-1-[(7-(2,4,6-trichlorophenyl)-2,3-dihydro-1-benzofuran-2-yl]methanamine,
(+)-{[7-(2,6-dimethylphenyl)-5-fluoro-2,3-dihydro-1-benzofuran-2-yl]methyl}methylamine,
(−)-{[7-(2,6-dimethylphenyl)-5-fluoro-2,3-dihydro-1-benzofuran-2-yl]methyl}methylamine,
(−)-{[7-(2,6-dimethylphenyl)-5-methoxy-2,3-dihydro-1-benzofuran-2-yl]methyl}methylamine,
(+)-{[7-(2,6-dimethylphenyl)-5-methoxy-2,3-dihydro-1-benzofuran-2-yl]methyl}methylamine,
(+)-{[5-chloro-7-(2,5-dichlorophenyl)-2,3-dihydro-1-benzofuran-2-yl]methyl}methylamine,
(−)-{[5-chloro-7-(2,5-dichlorophenyl)-2,3-dihydro-1-benzofuran-2-yl]methyl}methylamine,
(−)-{[5-chloro-7-(2,6-dimethylphenyl)-2,3-dihydro-1-benzofuran-2-yl]methyl}methylamine,
(+)-{[5-chloro-7-(2,6-dimethylphenyl)-2,3-dihydro-1-benzofuran-2-yl]methyl}methylamine,
(+)-{[7-(2,3-dimethoxyphenyl)-5-methyl-2,3-dihydro-1-benzofuran-2-yl]methyl}methylamine,
(−)-{[7-(2,3-dimethoxyphenyl)-5-methyl-2,3-dihydro-1-benzofuran-2-yl]methyl}methylamine,
(−)-{[7-(2,3-dimethoxyphenyl)-5-fluoro-2,3-dihydro-1-benzofuran-2-yl]methyl}methylamine, or
(+)-{[7-(2,3-dimethoxyphenyl)-5-fluoro-2,3-dihydro-1-benzofuran-2-yl]methyl}methylamine;
or a pharmaceutically acceptable salt thereof.

17. The method of claim 1, wherein the cognitive disorder is ADD or ADHD.

18. The method of claim 17, wherein the mammal is a pediatric patient.

19. The method of claim 1, wherein the cognitive disorder is a learning disorder.

20. The method of claim 18, wherein the learning disorder is autism, dyslexia, Asperger's syndrome, a specific learning disability, dysgraphia, dyscalculia, dyspraxia, visual perceptual deficit, or auditory perceptual deficit.

21. The method of claim 1, wherein the cognitive disorder is an impulsivity disorder.

22. The method of claim 30, wherein the impulsivity disorder is borderline personality disorder, a disruptive behavior disorder, an impulse control disorders, or Tourette's Syndrome.

23. The method of claim 1, wherein the cognitive disorder is a behavioral addiction or addictive disorder.

24. The method of claim 32, wherein the behavioral addiction or addictive disorder is gambling, sex addiction, eating disorders, spending addiction, rage/anger, workaholism, exercise addiction, a risk taking addiction, or perfectionism.

25. The method of claim 1, further comprising administering an additional therapeutic agent selected from an acetylcholinesterase inhibitor, galantamine, a neuroprotective agent, a therapeutic agent for treating ADD/ADHD.

26. The method of claim 34, wherein the additional agent is selected from donepezil hydrochloride, galantamine, memantine, methylphenidate, atomoxetine, or amphetamine/dextroamphetamine.

27. A method for treating PMS or PMDD, or one or more symptoms associated with PMS or PMDD, in a patient, comprising administering to said patient a therapeutically effective amount of a compound of formula I: 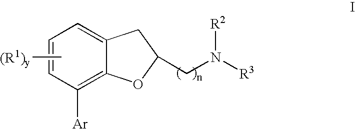
or a pharmaceutically acceptable salt thereof, wherein:
n is one or two;
each of R² and R³ is independently hydrogen, methyl, ethyl, 2-fluoroethyl, 2,2-difluoroethyl or cyclopropyl;
each R¹ is independently hydrogen, halogen, OH, lower alkyl, lower alkoxy, lower haloalkyl, lower haloalkoxy, or CN;
Ar is thienyl, furyl, pyridyl, or phenyl, wherein Ar is optionally substituted with one or more R^{x} subsituents;
each R^{x} is independently selected from halogen, OH, lower alkyl, lower alkoxy, lower haloalkyl, lower haloalkoxy, or CN; and
n is one or two.

28. The method of claim 36, wherein said symptom is one or more of irritability, depressed mood, anxiety, sleep disturbance, difficulty concentrating, angry outbursts, breast tenderness and bloating.

29. The method of claim 37, further comprising administering to said patient a selective serotonin reuptake inhibitor.

30. The method of claim 38, wherein said selective serotonin reuptake inhibitor is fluoxetine, venlafaxine, paroxetine, duloxetine, or sertraline.
